(19) United States
(12) Patent Application Publication
(10) Pub. No.: US 2015/0045245 A1
(43) Pub. Date: Feb. 12, 2015

(54) BIOMARKERS AND TEST PANELS USEFUL IN SYSTEMIC INFLAMMATORY CONDITIONS

(71) Applicant: BIOCARTIS NV, Mechelen (BE)

(72) Inventors: Griet Vanpoucke, Ingooigem (BE); Katleen Verleysen, Laarne (BE); Yven Van Herrewege, Laarne (BE)

(73) Assignee: BIOCARTIS NV, Mechelen (BE)

(21) Appl. No.: 14/363,068

(22) PCT Filed: Dec. 7, 2012
(86) PCT No.: PCT/EP2012/074806

§ 371 Date: Jun. 5, 2014

(30) Foreign Application Priority Data
Dec. 8, 2011 (EP) 11192666.3

(51) Int. Cl. G01N 33/68

(52) U.S. Cl. CPC G01N 33/6893; G01N 2800/7095; G01N 2800/52; G01N 2800/26; G01N 2333/705; G01N 2333/47; G01N 2333/91097; G01N 2333/7155; G01N 2333/96433; G01N 2333/70542; G01N 2333/96425; G01N 2333/70525; G01N 2333/70535; G01N 2333/4727; G01N 2333/916; G01N 2333/75
USPC 506/9; 435/7.92; 435/29; 435/6.11; 435/6.12; 435/7.21; 436/501; 436/86; 506/12; 506/18

(57) ABSTRACT

The application discloses new biomarkers or test panels useful in systemic inflammatory conditions such as sepsis; methods for the diagnosis, prediction, prognosis and/or monitoring systemic inflammatory conditions such as sepsis based on measuring said biomarkers or test panels; and related kits and devices.

### FIELD OF THE INVENTION

The invention relates generally to biomarkers and test panels, more particularly to protein- and/or peptide-based biomarkers and test panels, useful in medical conditions, specifically useful in systemic inflammatory conditions such as sepsis, more specifically useful for the diagnosis, prediction, prognosis and/or monitoring of systemic inflammatory conditions such as sepsis in subjects. The invention further concerns methods, uses, kits and devices involving or related to the biomarkers and test panels.

### BACKGROUND OF THE INVENTION

In many diseases and conditions, a favourable outcome of prophylactic and/or therapeutic treatments is strongly correlated with early and/or accurate prediction, diagnosis, prognosis and/or monitoring of a disease or condition. Therefore, there exists a continuous need for additional and preferably improved manners for early and/or accurate prediction, diagnosis, prognosis and/or monitoring of diseases and conditions to guide the treatment choices.

Sepsis or blood poisoning is a life-threatening syndrome characterized by a systemic host response to infection, which can cause organ failure and death in severe cases. Sepsis accounts for over 10% of intensive care unit (ICU) admissions and is the leading cause of death in the non-coronary intensive care unit. Each year over 750,000 new cases are detected in the USA alone, with a mortality rate reaching nearly 30%, thereby ranking sepsis in the top ten causes of death. The total annual treatment costs in the USA amount to more than $16 billion and are still rising. Early goal-directed therapy can significantly reduce sepsis mortality validating the benefit of early identification of the syndrome and aggressive management. Early diagnosis and appropriate therapy of sepsis is a daily challenge in intensive care units. In order to enable a meaningful impact on individual patient outcome, reliable biomarkers for the early and/or accurate detection of sepsis are highly needed. Equally important are novel biomarkers for determining which patients are at increased risk to develop severe sepsis, in order to facilitate early intervention.

For the appropriate management of patients presenting with Systemic Inflammatory Response Syndrome (SIRS) it is important to be able to distinguish between infectious and non-infectious causes as early as possible. Mortality increases by approximately 5% per hour when the start of appropriate antibiotics therapy is delayed. Blood culture, the gold standard to identify infection causative organisms, is hampered by low sensitivity and even when positive, the results are obtained too late to influence clinical decision making in the early hours after onset of sepsis. PCR based tests to detect the pathogens are being introduced into the clinic; however they are criticized because of disappointing sensitivity and their labor-intensive use. The only FDA-approved marker for sepsis is Procalcitonin (PCT) and it is proposed as a diagnostic and prognostic marker of sepsis and as a guide for antibiotics use (Schuetz et al. BMC Med., 2011, vol. 9, 107). Elevated levels of PCT have been associated with severe bacterial infections among children and adults, although its performance as a marker for early infection or sepsis is not optimal.

In the past decade there have been a large number of reports on the use of PCT as a serum marker of systemic infection and sepsis. Results of these studies vary however greatly with diagnostic performances ranging from good to comparatively much less satisfactory (Becker et al. Crit. Care Med., 2008, vol. 36(3), 941-52). PCT diagnostic performance tends to be largely dependent on the study population and clinical milieu. Several non-infectious inflammatory conditions such as burn injury, major trauma or extensive surgery and pancreatitis lead to elevated PCT levels, complicating the evaluation and use of PCT in critically ill patients. A number of meta-analyses on the accuracy of PCT for sepsis diagnosis have been published, with contradictory conclusions. Tang et al. (Lancet Infect. Dis., 2007, vol. 7(3), 210-7) estimated the diagnostic sensitivity and specificity of PCT both to be around 71% and argue against the widespread use of the test in critical care settings.

Clearly there remains the need to provide for further and/or improved markers for use in conditions involving systemic inflammation such as sepsis, as well as to potentially further improve the diagnostic accuracy of PCT.

### SUMMARY OF THE INVENTION

Having conducted extensive experiments and tests, the inventors identified 50 biomarkers that may be employed for evaluating various aspects of systemic inflammatory conditions such as sepsis in subjects.

In particular, as shown in the experimental section, which includes exemplary data pertaining to certain embodiments of the present invention, using serum or plasma samples of more than 150 patients presenting with signs of systemic inflammatory response syndrome (SIRS) and suspicion of sepsis, the inventors realised that the quantity of the following protein- and/or peptide-based markers in said samples displayed a behaviour predictive and/or indicative of certain clinical outcomes that are highly relevant in the context of systemic inflammatory conditions: proteinase 3 (PRTN3), macrophage mannose receptor 1 (MRC1), exostoses (multiple) 2 (EXT2), interleukin 1 receptor type II (IL1R2), pentraxin 3 long (PTX3), mannosyl-oligosaccharide 1,2-alpha-mannosidase IA (MA1A1), Acyl-CoA-binding protein (ACBP), vesicular integral-membrane protein VIP36 (LMAN2), neuronal acetylcholine receptor subunit alpha-7 (ACHA7), cyclic AMP-dependent transcription factor ATF-6 alpha (ATF6A), Beta-1,4-galactosyltransferase 1 (B4GT1), cathelicidin antimicrobial peptide (CAMP), Golgi membrane protein 1 (GOLM1), Nidogen-1 (NID1), matrix metallopeptidase 3 (MMP3), lipopolysaccharide-binding protein (LBP), fibulin 1 (FBLN1), polymeric-immunoglobulin receptor (PIGR), TIMP metalloproteinase inhibitor 1 (TIMP1), glycosylphosphatidylinositol specific phospholipase D1 (PHLD), angiotensinogen (ANGT), carboxypeptidase N catalytic chain (CBPN), chitinase-3-like protein 1 (CH3L1), macrophage colony-stimulating factor 1 (CSF1), dystryglycan (DAG1), fibrillin-1 (FBN1), fibrinogen-like 1 (FGL1), glutathione synthetase (GSHB), intercellular adhesion molecule 1 (ICAM1), lumican (LUM), S100 calcium binding protein A9 (S10A9), serum amyloid A protein (SAA), serglycin (SRGN), Vascular cell adhesion protein 1 (VCAM1), calumenin (CALU), echinoderm microtubule associated protein like 3 (EMAL3), Rho GDP dissociation inhibitor beta (GDIR2), guanylate cyclase activator 2B (GUC2B), heat shock 70 kDa protein 8 (HSP7C), interleukin 13 receptor alpha 1 (I13R1), moesin (MOES), protein disulfide isomerase family A member 6 (PDIA6), proteasome subunit alpha type 3 (PSA3), protein tyrosine phosphatase receptor type G (PTPRG) and S100 calcium binding protein A8 (S10A8). These proteins may be encoded respectively by PRTN3, MRC1, EXT2, IL1R2, PTX3, MAN1A1, DBI, LMAN2, CHRNA7, ATF6, B4GALT1, CAMP, GOLM1, NID1, MMP3, LBP, FBLN1, PIGR, TIMP1, GPLD1, AGT, CPN1, CHI3L1, CSF1, DAG1, FBN1, FGL1, GSS, ICAM1, LUM, S100A9, SAA, SRGN, VCAM1, CALU, EML3, ARHGDIB, GUCA2B, HSPA8, IL13RA1, MSN, PDIA6, PSMA3, PTPRG and S100A8 genes.

The inventors further understood that proteinase 3 (PRTN3) is a hematopoietic serine protease stored in large quantities in neutrophil cytoplasmic azurophilic granules. Two other serine proteases, cathepsin G (CATG) and neutrophil elastase (ELNE), belong to major components of neutrophil azurophilic granules and participate in the non-oxidative pathway of intracellular and extracellular pathogen destruction. Based on the similarity of cellular localisation and biological function of proteinase 3, cathepsin G and neutrophil elastase, the inventors postulated that each of cathepsin G and neutrophil elastase also displays a behaviour predictive and/or indicative of certain clinical outcomes that are highly relevant in the context of systemic inflammatory conditions.

In an aspect, the present invention thus provides the use of any one or more of PRTN3, MRC1, EXT2, ILIR2, PTX3, MA1A1, ACBP, LMAN2, ACHA7, ATF6A, B4GT1, CAMP, GOLM1, NID1, MMP3, LBP, FBLN1, PIGR, TIMP1, PHLD, ANGT, CBPN, CH3L1, CSF1, DAG1, FBN1, FGL1, GSHB, ICAM1, LUM, S10A9, SAA, SRGN, VCAM1, CALU, EMAL3, GDIR2, GUC2B, HSP7C, I13R1, MOES, PDIA6, PSA3, PTPRG, S10A8, CATG, and ELNE, or a fragment thereof, as a biomarker, preferably as a biomarker for a systemic inflammatory condition, more preferably as a biomarker for the diagnosis, prediction, prognosis and/or monitoring of a systemic inflammatory condition in a subject. In some preferred embodiments, said systemic inflammatory condition may be sepsis. In other embodiments, said systemic inflammatory condition may be SIRS. The present uses may be adequately qualified as in vitro or ex vivo uses, in that they apply particular in vitro or ex vivo processing and analysis on a sample obtained from a subject.

Certain embodiments provide the use of any one or more of PRTN3, MRC1, EXT2, ILIR2, PTX3, MA1A1, ACBP, LMAN2, ACHA7, ATF6A, B4GT1, CAMP, GOLM1, NID1, MMP3, LBP, FBLN1, PIGR, TIMP1, PHLD, ANGT, CBPN, CH3L1, CSF1, DAG1, FBN1, FGL1, GSHB, ICAM1, LUM, S10A9, SAA, SRGN, VCAM1, CALU, EMAL3, GDIR2, GUC2B, HSP7C, I13R1, MOES, PDIA6, PSA3, PTPRG, and S10A8, or a fragment thereof, as a biomarker, preferably as a biomarker for a systemic inflammatory condition, more preferably as a biomarker for the diagnosis, prediction, prognosis and/or monitoring of a systemic inflammatory condition in a subject. In some preferred embodiments, said systemic inflammatory condition may be sepsis. In other embodiments, said systemic inflammatory condition may be SIRS.

Advantageously, early and dependable information on the type and characteristics of a systemic inflammatory condition in a subject resulting from such uses can provide valuable guidance to medical practitioners as to timely commencement of a suitable therapeutic intervention in these patients. Prompt intervention in these patients, who are often critically ill, is highly relevant.

Also provided is use of any one or more of PRTN3, MRC1, EXT2, ILIR2, PTX3, MA1A1, ACBP, LMAN2, ACHA7, ATF6A, B4GT1, CAMP, GOLM1, NID1, MMP3, LBP, FBLN1, PIGR, TIMP1, PHLD, ANGT, CBPN, CH3L1, CSF1, DAG1, FBN1, FGL1, GSHB, ICAM1, LUM, S10A9, SAA, SRGN, VCAM1, CALU, EMAL3, GDIR2, GUC2B, HSP7C, I13R1, MOES, PDIA6, PSA3, PTPRG, S10A8, CATG, and ELNE, or a fragment thereof, for the diagnosis, prediction, prognosis and/or monitoring of a systemic inflammatory condition in a subject. In some preferred embodiments, said systemic inflammatory condition may be sepsis. In other embodiments, said systemic inflammatory condition may be SIRS.

Certain embodiments provide use of any one or more of PRTN3, MRC1, EXT2, IL1R2, PTX3, MA1A1, ACBP, LMAN2, ACHA7, ATF6A, B4GT1, CAMP, GOLM1, NID1, MMP3, LBP, FBLN1, PIGR, TIMP1, PHLD, ANGT, CBPN, CH3L1, CSF1, DAG1, FBN1, FGL1, GSHB, ICAM1, LUM, S10A9, SAA, SRGN, VCAM1, CALU, EMAL3, GDIR2, GUC2B, HSP7C, I13R1, MOES, PDIA6, PSA3, PTPRG, and S10A8, or a fragment thereof, for the diagnosis, prediction, prognosis and/or monitoring of a systemic inflammatory condition in a subject. In some preferred embodiments, said systemic inflammatory condition may be sepsis. In other embodiments, said systemic inflammatory condition may be SIRS.

In another aspect, the present invention provides a method for the diagnosis, prediction, prognosis and/or monitoring of a systemic inflammatory condition in a subject, wherein the method comprises measuring the quantity of any one or more markers selected from the group consisting of PRTN3, MRC1, EXT2, IL1R2, PTX3, MA1A1, ACBP, LMAN2, ACHA7, ATF6A, B4GT1, CAMP, GOLM1, NID1, MMP3, LBP, FBLN1, PIGR, TIMP1, PHLD, ANGT, CBPN, CH3L1, CSF1, DAG1, FBN1, FGL1, GSHB, ICAM1, LUM, S10A9, SAA, SRGN, VCAM1, CALU, EMAL3, GDIR2, GUC2B, HSP7C, I13R1, MOES, PDIA6, PSA3, PTPRG, S10A8, CATG, and ELNE, or a fragment thereof, in a sample from the subject. In some preferred embodiments, said systemic inflammatory condition may be sepsis. In other embodiments, said systemic inflammatory condition may be SIRS.

Certain embodiments provide a method for the diagnosis, prediction, prognosis and/or monitoring of a systemic inflammatory condition in a subject, wherein the method comprises measuring the quantity of any one or more markers selected from the group consisting of PRTN3, MRC1, EXT2, ILIR2, PTX3, MA1A1, ACBP, LMAN2, ACHA7, ATF6A, B4GT1, CAMP, GOLM1, NID1, MMP3, LBP, FBLN1, PIGR, TIMP1, PHLD, ANGT, CBPN, CH3L1, CSF1, DAG1, FBN1, FGL1, GSHB, ICAM1, LUM, S10A9, SAA, SRGN, VCAM1, CALU, EMAL3, GDIR2, GUC2B, HSP7C, I13R1, MOES, PDIA6, PSA3, PTPRG, and S10A8, or a fragment thereof, in a sample from the subject. In some preferred embodiments, said systemic inflammatory condition may be sepsis. In other embodiments, said systemic inflammatory condition may be SIRS.

Particularly provided is the method for the diagnosis, prediction, prognosis and/or monitoring of the systemic inflammatory condition in the subject, wherein the examination phase of the method comprises measuring the quantity of said one or more markers. Hence, as used throughout this specification, measuring the quantity of any one or more biomarker(s) in a sample from a subject may particularly denote that the examination phase of a method comprises measuring the quantity of said one or more biomarker(s) in the sample from the subject. One understands that methods for the diagnosis, prediction, prognosis and/or monitoring of diseases and conditions generally comprise an examination phase in which data is collected from and/or about the subject.

Advantageously, early and dependable information on the type and characteristics of a systemic inflammatory condition in a subject resulting from such methods can provide valuable guidance to medical practitioners as to timely commencement of a suitable therapeutic intervention in these patients. Prompt intervention in these patients, who are often critically ill, is highly relevant.

Without any limitation, in certain embodiments of the herein taught uses and methods, said one or more markers may be selected from the group consisting of PRTN3, MRC1, EXT2, ILIR2, MA1A1, ACBP, LMAN2, ACHA7, ATF6A, B4GT1, CAMP, GOLM1, NID1, MMP3, FBLN1, PIGR, TIMP1, PHLD, ANGT, CBPN, CH3L1, DAG1, FBN1, FGL1, GSHB, ICAM1, LUM, SAA, SRGN, VCAM1, CALU, EMAL3, GDIR2, GUC2B, HSP7C, I13R1, MOES, PDIA6, PSA3, PTPRG, CATG, and ELNE. Without any limitation, in certain embodiments of the herein taught uses and methods, said one or more markers may be selected from the group consisting of PRTN3, MRC1, EXT2, IL1R2, MA1A1, ACBP, LMAN2, ACHA7, ATF6A, B4GT1, CAMP, GOLM1, NID1, MMP3, FBLN1, PIGR, TIMP1, PHLD, ANGT, CBPN, CH3L1, DAG1, FBN1, FGL1, GSHB, ICAM1, LUM, SAA, SRGN, VCAM1, CALU, EMAL3, GDIR2, GUC2B, HSP7C, I13R1, MOES, PDIA6, PSA3 and PTPRG. In such embodiments, the evaluation of said one or more of the foregoing markers may be optionally combined with the evaluation of one or more markers selected from the group consisting of LBP, PTX3, CSF1, S10A9 and S10A8.

In particularly preferred embodiments, the present biomarkers may be protein-, polypeptide- or peptide-based biomarkers. Particularly preferably, such protein-, polypeptide- or peptide-based biomarkers can be detected in blood, plasma or serum samples.

In preferred embodiments, the present method for the diagnosis, prediction and/or prognosis of a systemic inflammatory condition in a subject may comprise the steps of: (i) measuring the quantity of any one or more markers selected from the group consisting of PRTN3, MRC1, EXT2, ILIR2, PTX3, MA1A1, ACBP, LMAN2, ACHA7, ATF6A, B4GT1, CAMP, GOLM1, NID1, MMP3, LBP, FBLN1, PIGR, TIMP1, PHLD, ANGT, CBPN, CH3L1, CSF1, DAG1, FBN1, FGL1, GSHB, ICAM1, LUM, S10A9, SAA, SRGN, VCAM1, CALU, EMAL3, GDIR2, GUC2B, HSP7C, I13R1, MOES, PDIA6, PSA3, PTPRG, S10A8, CATG, and ELNE, or a fragment thereof, or of any one or more markers selected from the group consisting of PRTN3, MRC1, EXT2, ILIR2, PTX3, MA1A1, ACBP, LMAN2, ACHA7, ATF6A, B4GT1, CAMP, GOLM1, NID1, MMP3, LBP, FBLN1, PIGR, TIMP1, PHLD, ANGT, CBPN, CH3L1, CSF1, DAG1, FBN1, FGL1, GSHB, ICAM1, LUM, S10A9, SAA, SRGN, VCAM1, CALU, EMAL3, GDIR2, GUC2B, HSP7C, I13R1, MOES, PDIA6, PSA3, PTPRG and S10A8, or a fragment thereof, in the sample from the subject; (ii) comparing the quantity of said one or more markers measured in (i) with a reference value of the quantity of said one or more markers, said reference value representing a known diagnosis, prediction and/or prognosis of the systemic inflammatory condition; (iii) finding a deviation or no deviation of the quantity of said one or more markers measured in (i) from said reference value; and (iv) attributing said finding of deviation or no deviation to a particular diagnosis, prediction and/or prognosis of the systemic inflammatory condition in the subject. In some preferred embodiments, said systemic inflammatory condition may be sepsis. In other embodiments, said systemic inflammatory condition may be SIRS.

In a further aspect the invention relates to a system comprising:
a computer data repository that comprises a reference value of the quantity of one or more markers selected from the group consisting of PRTN3, MRC1, EXT2, IL1R2, PTX3, MA1A1, ACBP, LMAN2, ACHA7, ATF6A, B4GT1, CAMP, GOLM1, NID1, MMP3, LBP, FBLN1, PIGR, TIMP1, PHLD, ANGT, CBPN, CH3L1, CSF1, DAG1, FBN1, FGL1, GSHB, ICAM1, LUM, S10A9, SAA, SRGN, VCAM1, CALU, EMAL3, GDIR2, GUC2B, HSP7C, I13R1, MOES, PDIA6, PSA3, PTPRG, S10A8, CATG, and ELNE, or a fragment thereof, or of one or more markers selected from the group consisting of PRTN3, MRC1, EXT2, ILIR2, PTX3, MA1A1, ACBP, LMAN2, ACHA7, ATF6A, B4GT1, CAMP, GOLM1, NID1, MMP3, LBP, FBLN1, PIGR, TIMP1, PHLD, ANGT, CBPN, CH3L1, CSF1, DAG1, FBN1, FGL1, GSHB, ICAM1, LUM, S10A9, SAA, SRGN, VCAM1, CALU, EMAL3, GDIR2, GUC2B, HSP7C, I13R1, MOES, PDIA6, PSA3, PTPRG and S10A8, or a fragment thereof, said reference value representing a known diagnosis, prediction and/or prognosis of a systemic inflammatory condition; and
a computer system programmed to access the data repository and to use information from the data repository in combination with information on the quantity of said one or more markers selected from the group consisting of PRTN3, MRC1, EXT2, IL1R2, PTX3, MA1A1, ACBP, LMAN2, ACHA7, ATF6A, B4GT1, CAMP, GOLM1, NID1, MMP3, LBP, FBLN1, PIGR, TIMP1, PHLD, ANGT, CBPN, CH3L1, CSF1, DAG1, FBN1, FGL1, GSHB, ICAM1, LUM, S10A9, SAA, SRGN, VCAM1, CALU, EMAL3, GDIR2, GUC2B, HSP7C, I13R1, MOES, PDIA6, PSA3, PTPRG, S10A8, CATG, and ELNE, or a fragment thereof, or said one or more markers selected from the group consisting of PRTN3, MRC1, EXT2, ILIR2, PTX3, MA1A1, ACBP, LMAN2, ACHA7, ATF6A, B4GT1, CAMP, GOLM1, NID1, MMP3, LBP, FBLN1, PIGR, TIMP1, PHLD, ANGT, CBPN, CH3L1, CSF1, DAG1, FBN1, FGL1, GSHB, ICAM1, LUM, S10A9, SAA, SRGN, VCAM1, CALU, EMAL3, GDIR2, GUC2B, HSP7C, I13R1, MOES, PDIA6, PSA3, PTPRG and S10A8, or a fragment thereof, in a sample from a subject, to make a diagnosis, prediction and/or prognosis of a systemic inflammatory condition in the subject. In some preferred embodiments, said systemic inflammatory condition may be sepsis. In other embodiments, said systemic inflammatory condition may be SIRS.

Related embodiments of the invention concern a method for making diagnosis, prediction and/or prognosis of a systemic inflammatory condition in a subject comprising:
(i) receiving data representative of values of the quantity of any one or more markers selected from the group consisting of PRTN3, MRC1, EXT2, IL1R2, PTX3, MA1A1, ACBP, LMAN2, ACHA7, ATF6A, B4GT1, CAMP, GOLM1, NID1, MMP3, LBP, FBLN1, PIGR, TIMP1, PHLD, ANGT, CBPN, CH3L1, CSF1, DAG1, FBN1, FGL1, GSHB, ICAM1, LUM, S10A9, SAA, SRGN, VCAM1, CALU, EMAL3, GDIR2, GUC2B, HSP7C, I13R1, MOES, PDIA6, PSA3, PTPRG, S10A8, CATG, and ELNE, or a fragment thereof, or any one or more markers selected from the group consisting of PRTN3, MRC1, EXT2, IL1R2, PTX3, MA1A1, ACBP, LMAN2, ACHA7, ATF6A, B4GT1, CAMP, GOLM1, NID1, MMP3, LBP, FBLN1, PIGR, TIMP1, PHLD, ANGT, CBPN, CH3L1, CSF1, DAG1, FBN1, FGL1, GSHB, ICAM1, LUM, S10A9, SAA, SRGN, VCAM1, CALU, EMAL3, GDIR2, GUC2B, HSP7C, I13R1, MOES, PDIA6, PSA3, PTPRG and S10A8, or a fragment thereof, in a sample from the subject;
(ii) accessing a data repository on a computer, said data repository comprising a reference value of the quantity of said one or more markers, said reference value representing a known diagnosis, prediction and/or prognosis of a systemic inflammatory condition; and
(iii) comparing the data as received in (i) with the reference value in the data repository on the computer, thereby making a diagnosis, prediction and/or prognosis of a systemic inflammatory condition in the subject.

In certain embodiments, the determination of what action is to be taken, e.g., by a clinician, in view of said diagnosis, prediction and/or prognosis is performed by a (the) computer. In certain embodiments, a (the) computer reports (i.e., generates an electronic report of) the action to be taken, preferably substantially in real time.

Throughout the present disclosure, methods and uses for the prediction or prognosis of any one disease or condition as taught herein can inter alia allow the prediction of the occurrence of the disease or condition, or make a prognosis of the progression, aggravation, alleviation or recurrence of the disease or condition or response to treatment or to other external or internal factors, situations or stressors, etc. As intended herein, a reference to prediction of any disease or condition also specifically includes prediction of the probability, risk or chance of a subject to develop the disease or condition.

In further preferred embodiments, the present method for monitoring a systemic inflammatory condition in a subject may comprise the steps of: (i) measuring the quantity of any one or more markers selected from the group consisting of PRTN3, MRC1, EXT2, ILIR2, PTX3, MA1A1, ACBP, LMAN2, ACHA7, ATF6A, B4GT1, CAMP, GOLM1, NID1, MMP3, LBP, FBLN1, PIGR, TIMP1, PHLD, ANGT, CBPN, CH3L1, CSF1, DAG1, FBN1, FGL1, GSHB, ICAM1, LUM, S10A9, SAA, SRGN, VCAM1, CALU, EMAL3, GDIR2, GUC2B, HSP7C, I13R1, MOES, PDIA6, PSA3, PTPRG, S10A8, CATG, and ELNE, or a fragment thereof, or of any one or more markers selected from the group consisting of PRTN3, MRC1, EXT2, IL1R2, PTX3, MA1A1, ACBP, LMAN2, ACHA7, ATF6A, B4GT1, CAMP, GOLM1, NID1, MMP3, LBP, FBLN1, PIGR, TIMP1, PHLD, ANGT, CBPN, CH3L1, CSF1, DAG1, FBN1, FGL1, GSHB, ICAM1, LUM, S10A9, SAA, SRGN, VCAM1, CALU, EMAL3, GDIR2, GUC2B, HSP7C, I13R1, MOES, PDIA6, PSA3, PTPRG and S10A8, or a fragment thereof, in samples from the subject from two or more successive time points; (ii) comparing the quantity of said one or more markers between the samples as measured in (i); (iii) finding a deviation or no deviation of the quantity of said one or more markers between the samples as compared in (ii); (iv) attributing said finding of deviation or no deviation to a change in the systemic inflammatory condition in the subject between the two or more successive time points. Such method thus allows the monitoring of the systemic inflammatory condition in a subject over time. In some preferred embodiments, said systemic inflammatory condition may be sepsis. In other embodiments, said systemic inflammatory condition may be SIRS.

Throughout the present disclosure, methods and uses for monitoring any one disease or condition as taught herein can inter alia allow the monitoring of the progression, aggravation, alleviation or recurrence of the disease or condition, or response to treatment or to other external or internal factors, situations or stressors, etc. As intended herein, a reference to prediction of any disease or condition also specifically includes monitoring change(s) in the probability, risk or chance of a subject to develop the disease or condition. Advantageously, such monitoring methods may be applied in the course of a medical treatment of the subject, preferably medical treatment aimed at alleviating the so-monitored disease or condition. Such monitoring may be comprised, e.g., in decision making whether a patient may be discharged, needs a change in treatment or needs further hospitalisation.

In certain preferred embodiments, methods and uses for monitoring any one disease or condition as taught herein, in particular systemic inflammatory condition, such as sepsis or SIRS, preferably sepsis, can be applied to monitor the effectiveness of therapy of the disease or condition, or to decide on initiation, continuation or discontinuation (ending) of the therapy. Suitable therapies in this connection may include, for example, therapy with anti-microbial agents, preferably anti-bacterial agents, such as antibiotics; analgesics; antipyretics; and anti-inflammatory drugs, such as non-steroidal anti-inflammatory drugs (NSAID). Any one or a combination of two or more such therapies may be used. Preferably, such therapy may be antibiotics therapy.

The above methods for the diagnosis, prediction, prognosis and/or monitoring of the systemic inflammatory condition in the subject may in certain embodiments also be applied to determine whether the subject is or is not—for example, still is, or is no longer—in need of a therapeutic or prophylactic (preventative) treatment of the systemic inflammatory condition. For example, a treatment may be particularly indicated where the methods allow for a conclusion that the subject has or is at risk of having the systemic inflammatory condition, or has a poor prognosis for the systemic inflammatory condition, such as for example organ failure, multiple organ dysfunction syndrome (MODS) or death, or displays a detrimental development of the systemic inflammatory condition. Without limitation, a patient with the systemic inflammatory condition upon admission to or during stay in a medical care centre such as ICU may be tested as taught herein for the necessity of continuing the treatment of said systemic inflammatory condition, and may be discharged when such treatment is no longer needed or is needed only to a given limited extent.

In certain embodiments, the invention relates to a method for treating a systemic inflammatory condition in a subject in need of said treatment, the method comprising the steps of:

(i) measuring the quantity of any one or more markers selected from the group consisting of PRTN3, MRC1, EXT2, ILIR2, PTX3, MA1A1, ACBP, LMAN2, ACHA7, ATF6A, B4GT1, CAMP, GOLM1, NID1, MMP3, LBP, FBLN1, PIGR, TIMP1, PHLD, ANGT, CBPN, CH3L1, CSF1, DAG1, FBN1, FGL1, GSHB, ICAM1, LUM, S10A9, SAA, SRGN, VCAM1, CALU, EMAL3, GDIR2, GUC2B, HSP7C, I13R1, MOES, PDIA6, PSA3, PTPRG, S10A8, CATG, and ELNE, or a fragment thereof, or of any one or more markers selected from the group consisting of PRTN3, MRC1, EXT2, ILIR2, PTX3, MA1A1, ACBP, LMAN2, ACHA7, ATF6A, B4GT1, CAMP, GOLM1, NID1, MMP3, LBP, FBLN1, PIGR, TIMP1, PHLD, ANGT, CBPN, CH3L1, CSF1, DAG1, FBN1, FGL1, GSHB, ICAM1, LUM, S10A9, SAA, SRGN, VCAM1, CALU, EMAL3, GDIR2, GUC2B, HSP7C, I13R1, MOES, PDIA6, PSA3, PTPRG and S10A8, or a fragment thereof, in a sample from the subject;
(ii) comparing the quantity of said one or more markers measured in (i) with a reference value of the quantity of said one or more markers, said reference value representing a known diagnosis, prediction and/or prognosis of the systemic inflammatory condition;
(iii) finding a deviation or no deviation of the quantity of said one or more markers measured in (i) from said reference value;
(iv) attributing said finding of deviation or no deviation to a particular diagnosis, prediction and/or prognosis of the systemic inflammatory condition in the subject;
(v) inferring from said particular diagnosis, prediction and/or prognosis of the systemic inflammatory condition in the subject the presence or absence of a need for a therapeutic or prophylactic treatment of the systemic inflammatory condition in the subject; and
(v) administering a therapeutically or prophylactically effective amount of an active pharmaceutical ingredient capable of treating the systemic inflammatory condition to said subject when the subject is in need of said treatment.

In some preferred embodiments, said systemic inflammatory condition may be sepsis. In other embodiments, said systemic inflammatory condition may be SIRS. Examples of active pharmaceutical ingredients capable of treating systemic inflammatory conditions may include, without limitation, anti-microbial agents, preferably anti-bacterial agents, such as antibiotics; analgesics; antipyretics; and anti-inflammatory drugs, such as non-steroidal anti-inflammatory drugs (NSAID). Any one or a combination of two or more may be used.

The inventors further realised that the markers disclosed herein may be particularly advantageously employed for evaluating certain preferred aspects or clinical outcomes of systemic inflammatory conditions.

Accordingly, certain preferred embodiments provide the use of any one or more of PRTN3, MRC1, EXT2, ILIR2, PTX3, MA1A1, ACBP, LMAN2, ACHA7, ATF6A, B4GT1, CAMP, GOLM1, NID1, MMP3, LBP, FBLN1, PIGR, TIMP1, PHLD, ANGT, CBPN, CH3L1, CSF1, DAG1, FBN1, FGL1, GSHB, ICAM1, LUM, S10A9, SAA, SRGN, VCAM1, CALU, EMAL3, GDIR2, GUC2B, HSP7C, I13R1, MOES, PDIA6, PSA3, PTPRG, S10A8, CATG, and ELNE, or a fragment thereof, or of any one or more of PRTN3, MRC1, EXT2, ILIR2, PTX3, MA1A1, ACBP, LMAN2, ACHA7, ATF6A, B4GT1, CAMP, GOLM1, NID1, MMP3, LBP, FBLN1, PIGR, TIMP1, PHLD, ANGT, CBPN, CH3L1, CSF1, DAG1, FBN1, FGL1, GSHB, ICAM1, LUM, S10A9, SAA, SRGN, VCAM1, CALU, EMAL3, GDIR2, GUC2B, HSP7C, I13R1, MOES, PDIA6, PSA3, PTPRG, and S10A8, or a fragment thereof, as a biomarker for the diagnosis of whether a subject presenting with one or more signs of systemic inflammatory response syndrome (SIRS) has infection-free SIRS or has sepsis. In other words, said use allows the diagnosis of whether a subject having systemic inflammatory response syndrome (SIRS) does or does not have an infection, hence, whether the SIRS is caused by an infection or not. Preferably but without limitation, such infection may be bacterial infection. In certain preferred but non-limiting embodiments, such use may allow the distinction of mild sepsis (i.e., sepsis without organ failure) from infection-free SIRS. In certain other preferred but non-limiting embodiments, such use may allow the distinction of severe sepsis (i.e., sepsis and failure of at least one organ) from infection-free SIRS. In yet other preferred but non-limiting embodiments, such use may allow the distinction of SIRS caused by bacterial infection, such as for example bacteraemia, from infection-free SIRS.

Further preferred embodiments provide a method for the diagnosis of whether a subject presenting with one or more signs of systemic inflammatory response syndrome (SIRS) has infection-free SIRS or has sepsis, wherein the method comprises measuring the quantity of any one or more markers selected from the group consisting of PRTN3, MRC1, EXT2, IL1R2, PTX3, MA1A1, ACBP, LMAN2, ACHA7, ATF6A, B4GT1, CAMP, GOLM1, NID1, MMP3, LBP, FBLN1, PIGR, TIMP1, PHLD, ANGT, CBPN, CH3L1, CSF1, DAG1, FBN1, FGL1, GSHB, ICAM1, LUM, S10A9, SAA, SRGN, VCAM1, CALU, EMAL3, GDIR2, GUC2B, HSP7C, I13R1, MOES, PDIA6, PSA3, PTPRG, S10A8, CATG, and ELNE, or a fragment thereof, or of any one or more markers selected from the group consisting of PRTN3, MRC1, EXT2, IL1R2, PTX3, MA1A1, ACBP, LMAN2, ACHA7, ATF6A, B4GT1, CAMP, GOLM1, NID1, MMP3, LBP, FBLN1, PIGR, TIMP1, PHLD, ANGT, CBPN, CH3L1, CSF1, DAG1, FBN1, FGL1, GSHB, ICAM1, LUM, S10A9, SAA, SRGN, VCAM1, CALU, EMAL3, GDIR2, GUC2B, HSP7C, I13R1, MOES, PDIA6, PSA3, PTPRG and S10A8, or a fragment thereof, in a sample from the subject. In other words, said method allows the diagnosis of whether a subject having systemic inflammatory response syndrome (SIRS) does or does not have an infection, hence, whether the SIRS is caused by an infection or not. Preferably but without limitation, such infection may be bacterial infection. In certain preferred but non-limiting embodiments, such methods may allow distinguishing mild sepsis (i.e., sepsis without organ failure) from infection-free SIRS. In certain other preferred but non-limiting embodiments, such methods may allow distinguishing severe sepsis (i.e., sepsis and failure of at least one organ) from infection-free SIRS. In yet other preferred but non-limiting embodiments, such method may allow the distinction of SIRS caused by bacterial infection, such as for example bacteraemia, from infection-free SIRS.

Such uses and methods advantageously allow an early discrimination between subjects with sepsis, i.e. SIRS with an infection, and subjects with SIRS but without an infection. This is of particular importance for instance in critically ill patients and more in particular in critically ill patients presenting with signs of SIRS.

Further preferred embodiments provide the use of any one or more of PRTN3, MRC1, EXT2, ILIR2, PTX3, MA1A1, ACBP, LMAN2, ACHA7, ATF6A, B4GT1, CAMP, GOLM1, NID1, MMP3, LBP, FBLN1, PIGR, TIMP1, PHLD, ANGT, CBPN, CH3L1, CSF1, DAG1, FBN1, FGL1, GSHB, ICAM1, LUM, S10A9, SAA, SRGN, VCAM1, CALU, EMAL3, GDIR2, GUC2B, HSP7C, I13R1, MOES, PDIA6, PSA3, PTPRG, S10A8, CATG, and ELNE, or a fragment thereof, or of any one or more of PRTN3, MRC1, EXT2, ILIR2, PTX3, MA1A1, ACBP, LMAN2, ACHA7, ATF6A, B4GT1, CAMP, GOLM1, NID1, MMP3, LBP, FBLN1, PIGR, TIMP1, PHLD, ANGT, CBPN, CH3L1, CSF1, DAG1, FBN1, FGL1, GSHB, ICAM1, LUM, S10A9, SAA, SRGN, VCAM1, CALU, EMAL3, GDIR2, GUC2B, HSP7C, I13R1, MOES, PDIA6, PSA3, PTPRG, and S10A8, or a fragment thereof, as a biomarker for the diagnosis, prediction and/or prognosis of the severity of the systemic inflammatory condition in a subject. In particularly preferred embodiments, said systemic inflammatory condition may be SIRS or sepsis, more preferably sepsis.

Related preferred embodiments provide a method for the diagnosis, prediction and/or prognosis of the severity of the systemic inflammatory condition in a subject, wherein the method comprises measuring the quantity of any one or more markers selected from the group consisting of PRTN3, MRC1, EXT2, IL1R2, PTX3, MA1A1, ACBP, LMAN2, ACHA7, ATF6A, B4GT1, CAMP, GOLM1, NID1, MMP3, LBP, FBLN1, PIGR, TIMP1, PHLD, ANGT, CBPN, CH3L1, CSF1, DAG1, FBN1, FGL1, GSHB, ICAM1, LUM, S10A9, SAA, SRGN, VCAM1, CALU, EMAL3, GDIR2, GUC2B, HSP7C, I13R1, MOES, PDIA6, PSA3, PTPRG, S10A8, CATG, and ELNE, or a fragment thereof, or of any one or more markers selected from the group consisting of PRTN3, MRC1, EXT2, ILIR2, PTX3, MA1A1, ACBP, LMAN2, ACHA7, ATF6A, B4GT1, CAMP, GOLM1, NID1, MMP3, LBP, FBLN1, PIGR, TIMP1, PHLD, ANGT, CBPN, CH3L1, CSF1, DAG1, FBN1, FGL1, GSHB, ICAM1, LUM, S10A9, SAA, SRGN, VCAM1, CALU, EMAL3, GDIR2, GUC2B, HSP7C, I13R1, MOES, PDIA6, PSA3, PTPRG and S10A8, or a fragment thereof, in a sample from the subject. In particularly preferred embodiments, said systemic inflammatory condition may be SIRS or sepsis, more preferably sepsis.

In certain embodiments, the diagnosis, prediction and/or prognosis of the severity of the systemic inflammatory condition in the subject may comprise or consist of the prediction of mortality in the subject or the prognosis that the systemic inflammatory condition will result in death of the subject. By means of example but not limitation, such prediction or prognosis may evaluate the prospect of death of the subject in a given time period from the sampling (i.e., from the time when the sample in which the biomarker(s) is to be tested is taken from the subject), such as for example within a month or within 4 weeks (28 days) from sampling.

In certain other embodiments, the diagnosis, prediction and/or prognosis of the severity of the systemic inflammatory condition in the subject may comprise or consist of the diagnosis, prediction and/or prognosis of organ failure or multi-organ dysfunction syndrome in the subject.

The inventors further realised that certain markers disclosed herein may be particularly well performing in and thus advantageously employed for evaluating certain preferred aspects or clinical outcomes of systemic inflammatory conditions.

Accordingly, the aforementioned uses or methods for the diagnosis of whether a subject presenting with one or more signs of SIRS has infection-free SIRS or has sepsis may particularly preferably employ any one or more of PRTN3, MRC1, EXT2, IL1R2, PTX3, ACBP, ATF6A, B4GT1, GOLM1, NID1, LBP, FBLN1, TIMP1, CH3L1, CSF1, FGL1, ICAM1, LUM, S10A9, SAA, VCAM1, PSA3, PTPRG, S10A8, GSHB, PIGR, CALU, PHLD, CATG, and ELNE, or a fragment thereof, or any one or more of PRTN3, MRC1, EXT2, ILIR2, PTX3, ACBP, ATF6A, B4GT1, GOLM1, NID1, LBP, FBLN1, TIMP1, CH3L1, CSF1, FGL1, ICAM1, LUM, S10A9, SAA, VCAM1, PSA3, PTPRG, and S10A8, or a fragment thereof, or any one or more of PRTN3, GSHB, PTX3, VCAM1, PSA3, NID1, GOLM1, ATF6A, ICAM1, PIGR, CALU, EXT2, PHLD, FGL1, IL1R2, CATG, and ELNE, or a fragment thereof, as the one or more biomarker.

Further, the aforementioned uses or methods for predicting mortality in the subject or the prognosis that the systemic inflammatory condition will result in death of the subject may particularly preferably employ any one or more of MRC1, EXT2, PTX3, B4GT1, CAMP, GOLM1, TIMP1, PHLD, CH3L1, GSHB, ICAM1, VCAM1, HSP7C, PSA3 and PTPRG, or a fragment thereof, as the one or more biomarker.

Moreover, the aforementioned uses or methods for diagnosis, prediction and/or prognosis of organ failure or multi-organ dysfunction syndrome in the subject may particularly preferably employ any one or more of MRC1, EXT2, IL1R2, PTX3, ACBP, B4GT1, NID1, TIMP1, CH3L1, FGL1, SAA and PSA3, or a fragment thereof, as the one or more biomarker.

Further, as illustrated in the experimental section, in an exemplary cohort PRTN3 levels were very significantly increased, namely about two-fold higher, in sepsis patients compared to SIRS patients.

Hence, preferred embodiments provide the use of any one or more of PRTN3, CATG, and ELNE, or a fragment thereof, more preferably the use of PRTN3, or a fragment thereof, as a biomarker for the diagnosis, prediction, prognosis and/or monitoring of a systemic inflammatory condition in a subject, preferably wherein the systemic inflammatory condition is sepsis or SIRS, more preferably sepsis, particularly preferably as a biomarker for the diagnosis of sepsis.

Further preferred embodiments relate to a method for the diagnosis, prediction, prognosis, and/or monitoring of a systemic inflammatory condition in a subject, preferably wherein the systemic inflammatory condition is sepsis or SIRS, more preferably sepsis, particularly preferably a method for the diagnosis of sepsis, wherein the method comprises measuring the quantity of any one or more of PRTN3, CATG, and ELNE, or a fragment thereof, more preferably of PRTN3, or a fragment thereof, in a sample from the subject.

In certain embodiments, the method for the diagnosis, prediction and/or prognosis of a systemic inflammatory condition in a subject, preferably wherein the systemic inflammatory condition is sepsis or SIRS, more preferably sepsis, particularly preferably a method for the diagnosis of sepsis, may comprise the steps of: (i) measuring the quantity of any one or more of PRTN3, CATG, and ELNE, or a fragment thereof, preferably of PRTN3, or a fragment thereof, in the sample from the subject; (ii) comparing the quantity of said any one or more of PRTN3, CATG, and ELNE, or a fragment thereof, or of said PRTN3, or a fragment thereof, measured in (i) with a reference value of the quantity of said any one or more of PRTN3, CATG, and ELNE, or a fragment thereof, or of said PRTN3, or a fragment thereof, said reference value representing a known diagnosis, prediction and/or prognosis of the systemic inflammatory condition; (iii) finding a deviation or no deviation of the quantity of said any one or more of PRTN3, CATG, and ELNE, or a fragment thereof, or of said PRTN3, or a fragment thereof, measured in (i) from said reference value; and (iv) attributing said finding of deviation or no deviation to a particular diagnosis, prediction and/or prognosis of the systemic inflammatory condition in the subject.

In certain embodiments, the method for monitoring the systemic inflammatory condition, preferably monitoring sepsis, preferably in the course of a medical treatment of the subject, may comprise the steps of: (i) measuring the quantity of any one or more of PRTN3, CATG, and ELNE, or a fragment thereof, preferably of PRTN3, or a fragment thereof, in samples from the subject from two or more successive time points; (ii) comparing the quantity of said any one or more of PRTN3, CATG, and ELNE, or a fragment thereof, or of said PRTN3, or a fragment thereof, between the samples as measured in (i); (iii) finding a deviation or no deviation of the quantity of said any one or more of PRTN3, CATG, and ELNE, or a fragment thereof, or of said PRTN3, or a fragment thereof, between the samples as compared in (ii); and (iv) attributing said finding of deviation or no deviation to a change in the systemic inflammatory condition, preferably sepsis, in the subject between the two or more successive time points.

Moreover, PRTN3, CATG and/or ELNE levels, particularly preferably PRTN3 levels, advantageously allow the discrimination of each one of the following:
sepsis patients without organ failure, i.e., mild sepsis, from subjects with infection-free SIRS;
sepsis patients with failure of at least one organ, i.e., severe sepsis, from subjects with infection-free SIRS;
patients having bacterial infection, such as for example bacteraemia, from subjects with infection-free SIRS.

Particularly preferably, PRTN3, CATG and/or ELNE levels, even more preferably PRTN3 levels, advantageously allow the discrimination of sepsis patients without organ failure, i.e., mild sepsis, from subjects with infection-free SIRS;

Hence, a particularly preferred embodiment provides the use of any one or more of PRTN3, CATG, and ELNE, or a fragment thereof, more preferably of PRTN3 or a fragment thereof, as a biomarker for the diagnosis of whether a subject presenting with one or more signs of SIRS has infection-free SIRS or has sepsis, such as for example mild sepsis. Further preferred embodiment provides a method for the diagnosis of whether a subject presenting with one or more signs of SIRS has infection-free SIRS or has sepsis, such as for example mild sepsis, wherein the method comprises measuring the quantity of any one or more of PRTN3, CATG, and ELNE, or a fragment thereof, more preferably of PRTN3 or a fragment thereof, in a sample from the subject.

As further shown in the experimental section, S10A9 or S10A8 had in this cohort a performance equal to PCT for detecting infection or sepsis in a patient, and showed better performance than PCT to detect mild sepsis.

Hence, another particularly preferred embodiment provides the use of any one or both of S10A9 or S10A8, or a fragment thereof, as a biomarker for the diagnosis of whether a subject presenting with one or more signs of SIRS has infection-free SIRS or has sepsis, such as preferably mild sepsis. Further preferred embodiment provides a method for the diagnosis of whether a subject presenting with one or more signs of SIRS has infection-free SIRS or has sepsis, such as for example mild sepsis, wherein the method comprises measuring the quantity of any one or both of S10A9 or S10A8, or a fragment thereof, in a sample from the subject.

Such uses or methods may for example measure the level of the S10A9 protein or polypeptide, or a fragment thereof, or the level of the S10A8 protein or polypeptide, or a fragment thereof, or separately or cumulatively the level of both S10A9 and S10A8 proteins or polypeptides, or fragments thereof. In certain embodiments, the uses or methods may measure the level of the heterodimer of S10A9 and S10A8 known as calprotectin. In yet other embodiments, the uses or methods may measure the level of the S10A8 protein or polypeptide, or a fragment thereof, which forms part of the calprotectin heterodimer, or which does not form part of the calprotectin heterodimer (i.e., ‘free’ S10A8), or separately or cumulatively the levels of both the S10A8 protein or polypeptide, or a fragment thereof, which forms and which does not form part of the calprotectin heterodimer. In yet other embodiments, the uses or methods may measure the level of the S10A9 protein or polypeptide, or a fragment thereof, which forms part of the calprotectin heterodimer, or which does not form part of the calprotectin heterodimer (i.e., ‘free’ S10A9), or separately or cumulatively the levels of both the S10A9 protein or polypeptide, or a fragment thereof, which forms and which does not form part of the calprotectin heterodimer.

As further shown in the experimental section, in an exemplary cohort the inventors have found that MRC1 levels were significantly higher in non-survivors compared with survivors after one month of follow-up both in patients with sepsis and in patients with SIRS.

Hence, another particularly preferred embodiment provides the use of MRC1 or a fragment thereof as a biomarker for the prediction of mortality in a subject having a systemic inflammatory condition, such as preferably having SIRS or sepsis, more preferably having sepsis, or for the prognosis that said systemic inflammatory condition, such as preferably SIRS or sepsis, more preferably sepsis, will result in death of the subject. A further preferred embodiment provides a method for the prediction of mortality in a subject having a systemic inflammatory condition, such as preferably having SIRS or sepsis, more preferably having sepsis, or for the prognosis that said systemic inflammatory condition, such as preferably SIRS or sepsis, more preferably sepsis, will result in death of the subject, wherein the method comprises measuring the quantity of MRC1 or a fragment thereof in a sample from the subject. Preferably but without limitation, such prediction or prognosis of mortality or death in the subject may be in a given time period from the sampling (i.e., from the time when the sample in which the biomarker(s) is to be tested is taken from the subject), such as for example within a month or within 4 weeks (28 days) from sampling.

Hence, preferred embodiments provide the use of MRC1, or a fragment thereof, as a biomarker for the diagnosis, prediction, prognosis, and/or monitoring of a systemic inflammatory condition in a subject, preferably wherein the systemic inflammatory condition is sepsis or SIRS, more preferably sepsis, particularly preferably as a biomarker for the prognosis of sepsis.

Further preferred embodiments relate to a method for the diagnosis, prediction, prognosis and/or monitoring of a systemic inflammatory condition in a subject, preferably wherein the systemic inflammatory condition is sepsis or SIRS, more preferably sepsis, particularly preferably a method for the prognosis of sepsis, wherein the method comprises measuring the quantity of MRC1, or a fragment thereof, in a sample from the subject.

In certain embodiments, the method for the diagnosis, prediction and/or prognosis of a systemic inflammatory condition in a subject, preferably wherein the systemic inflammatory condition is sepsis or SIRS, more preferably sepsis, particularly preferably a method for the prognosis of sepsis, may comprise the steps of: (i) measuring the quantity of MRC1, or a fragment thereof, in the sample from the subject; (ii) comparing the quantity of said MRC1 measured in (i) with a reference value of the quantity of said MRC1, said reference value representing a known diagnosis, prediction and/or prognosis of the systemic inflammatory condition; (iii) finding a deviation or no deviation of the quantity of said MRC1 measured in (i) from said reference value; (iv) attributing said finding of deviation or no deviation to a particular diagnosis, prediction and/or prognosis of the systemic inflammatory condition in the subject.

In certain embodiments, the method for monitoring the systemic inflammatory condition, preferably monitoring sepsis, preferably in the course of a medical treatment of the subject, may comprise the steps of: (i) measuring the quantity of MRC1, or a fragment thereof, in samples from the subject from two or more successive time points; (ii) comparing the quantity of said MRC1 between the samples as measured in (i); (iii) finding a deviation or no deviation of the quantity of said MRC1 between the samples as compared in (ii); (iv) attributing said finding of deviation or no deviation to a change in the systemic inflammatory condition, preferably sepsis, in the subject between the two or more successive time points.

As also illustrated in the experimental section, any one of PTX3, IL1R2 and EXT2 showed at least equal performance to PCT to detect organ failure in patients presenting with signs of SIRS.

Hence, another particularly preferred embodiment provides the use of any one or more of PTX3, IL1R2 and EXT2, or a fragment thereof, as a biomarker for the diagnosis, prediction and/or prognosis of organ failure or multi-organ dysfunction syndrome in a subject having a systemic inflammatory condition, such as preferably having SIRS or sepsis, more preferably having sepsis. Further preferred embodiment provides a method for the diagnosis, prediction and/or prognosis of organ failure or multi-organ dysfunction syndrome in a subject having a systemic inflammatory condition, such as preferably having SIRS or sepsis, more preferably having sepsis, wherein the method comprises measuring the quantity of any one or more markers selected from the group consisting of PTX3, IL1R2 and EXT2, or a fragment thereof, in a sample from the subject.

As exemplified, EXT2 and PTX3 showed significantly higher levels in sepsis patients with organ failure compared to sepsis patient without organ failure. Consequently, EXT2 or PTX3, or a fragment thereof, may be particularly suitable as a biomarker for the diagnosis, prediction and/or prognosis of organ failure or multi-organ dysfunction syndrome in a subject having sepsis.

PTX3 or IL1R2 were found to be elevated in severe sepsis (i.e., sepsis and failure of at least one organ) patients compared to patients with SIRS. Hence, another particularly preferred embodiment provides the use of any one or both of PTX3 or IL1R2, or a fragment thereof, as a biomarker for the diagnosis of whether a subject presenting with one or more signs of SIRS has sepsis, preferably severe sepsis. A further preferred embodiment provides a method for the diagnosis of whether a subject presenting with one or more signs of SIRS has severe sepsis, wherein the method comprises measuring the quantity of any or both of PTX3 or IL1R2 or a fragment thereof, in a sample from the subject.

The application of uses and methods contemplated herein may be particularly valuable in subjects known or suspected to have a systemic inflammatory condition, such as sepsis or SIRS (for example but without limitation, known to have SIRS and suspected of having sepsis). For example, this may include critically ill patients, such as without limitation patients admitted to intensive care units (ICU) or emergency departments (ED), in whom the incidence of SIRS and sepsis, and more particularly sepsis, is known to be elevated. The uses and methods may be particularly helpful in critically ill patients admitted to ICU or ED with one or more signs of systemic inflammatory response syndrome (SIRS).

In embodiments, such critically ill patients may be admitted to ICU or ED with one or more of serious trauma, systemic inflammatory response syndrome (SIRS), chronic obstructive pulmonary disease (COPD), patients having undergone surgery, complications from surgery, medical shock, bacterial, fungal or viral infections, Acute Respiratory Distress Syndrome (ARDS), pulmonary and systemic inflammation, pulmonary tissue injury, severe pneumonia, respiratory failure, acute respiratory failure, respiratory distress, subarachnoidal hemorrhage (SAH), (severe) stroke, asphyxia, neurological conditions, organ dysfunction, single or multi-organ failure (MOF), poisoning and intoxication, severe allergic reactions and anaphylaxis, burn injury, acute cerebral hemorrhage or infarction, and any condition for which the patient requires assisted ventilation.

Particularly preferably, the patients such as critically ill patients, may present with one or more, more preferably two or more, signs of SIRS. Preferably, such signs may be selected from the group consisting of fever or hypothermia (temperature of 38.0° C. (100.4° F.) or more, or temperature of 36.0° C. (96.8° F.) or less); tachycardia (at least 90 beats per minute); tachypnea (at least 20 breaths per minute or PaCO₂ less than 4.3 kPa (32.0 mm Hg) or the need for mechanical ventilation); and an altered white blood cell (WBC) count of 12×10⁶ cells/mL or more, or an altered WBC count of 4×10⁶ cells/mL or less, or the presence of more than 10% band forms.

In particularly preferred embodiments, subjects as intended herein are mammalian, more preferably human.

In any the uses or methods as disclosed herein the measurement of any one or more markers selected from the group consisting of PRTN3, MRC1, EXT2, ILIR2, PTX3, MA1A1, ACBP, LMAN2, ACHA7, ATF6A, B4GT1, CAMP, GOLM1, NID1, MMP3, LBP, FBLN1, PIGR, TIMP1, PHLD, ANGT, CBPN, CH3L1, CSF1, DAG1, FBN1, FGL1, GSHB, ICAM1, LUM, S10A9, SAA, SRGN, VCAM1, CALU, EMAL3, GDIR2, GUC2B, HSP7C, I13R1, MOES, PDIA6, PSA3, PTPRG, S10A8, CATG, and ELNE, or of any one or more markers selected from the group consisting of PRTN3, MRC1, EXT2, IL1R2, PTX3, MA1A1, ACBP, LMAN2, ACHA7, ATF6A, B4GT1, CAMP, GOLM1, NID1, MMP3, LBP, FBLN1, PIGR, TIMP1, PHLD, ANGT, CBPN, CH3L1, CSF1, DAG1, FBN1, FGL1, GSHB, ICAM1, LUM, S10A9, SAA, SRGN, VCAM1, CALU, EMAL3, GDIR2, GUC2B, HSP7C, I13R1, MOES, PDIA6, PSA3, PTPRG, and S10A8, may be advantageously combined with the assessment of one or more other biomarkers or clinical parameters relevant for the diagnosis, prediction, prognosis and/or monitoring of the herein taught diseases or conditions, in particular systemic inflammatory conditions, more particularly SIRS or sepsis.

References throughout this specification to “other (bio)markers” or to “clinical parameters” generally encompasses such other markers or clinical parameters which are useful for the diagnosis, prediction, prognosis and/or monitoring of the herein taught diseases or conditions, in particular systemic inflammatory conditions, more particularly SIRS or sepsis. By means of example and not limitation, such other biomarkers include C-reactive protein (CRP), Procalcitonin (PCT), lactate, Cystatin C (CYTC), Neutrophil gelatinase-associated lipocalin (NGAL) and interleukin-6 (IL6), or a fragment thereof. Hence, the present uses and methods may further comprise measuring the presence or absence and/or quantity of one or more biomarkers selected from CRP, PCT, lactate, CYTC, NGAL and IL6, or a fragment thereof.

Hence, the present uses and methods may further comprise measuring (e.g., the examination phase of the methods may comprise measuring) the presence or absence and/or level of one or more such other biomarkers in the sample from the subject and/or may further comprise measuring the presence or absence and/or level of one or more such clinical parameters in the subject. Any known or yet unknown biomarkers or clinical parameters relevant for the diagnosis, prediction, prognosis and/or monitoring of the herein taught diseases or conditions may be used. In certain embodiments, the other biomarker(s) may be CRP, PCT, lactate, CYTC, NGAL and/or IL6, or a fragment thereof. In particularly preferred embodiments, the uses and methods may further comprise at least the evaluation of PCT or a fragment thereof.

In certain embodiments, the clinical parameters may be white blood cell (WBC) count, kidney function parameters such as serum creatinine and/or urine output, respiratory system function such as PaO2/FiO2, nervous system function preferably expressed as Glasgow coma scale, cardiovascular function preferably expressed as mean arterial pressure, liver function preferably expressed as bilirubin concentration, coagulation function preferably expressed as platelet concentration, etc.

It will be appreciated that the presence or absence and/or level of such other biomarkers or clinical parameters may be evaluated each separately and independently, or the presence or absence and/or level of such other biomarkers or clinical parameters may be included within subject profiles or reference profiles.

For example, in an embodiment, the uses and methods may comprise the evaluation of any one or more of PRTN3, CATG, and ELNE, or a fragment thereof, and at least the evaluation of PCT or a fragment thereof. For example, in a preferred embodiment, the uses and methods may comprise the evaluation of PRTN3 or a fragment thereof and at least the evaluation of PCT or a fragment thereof. This combination provides significant improvements over the use of PCT alone, particularly for diagnosing sepsis vs. infection-free SIRS, more particularly for diagnosing mild sepsis vs. infection-free SIRS.

The inventors interestingly found that combining evaluation of PTX3 with the evaluation of PCT greatly improves the performance of PTX3 to diagnose severe sepsis in subjects presenting with one or more signs of SIRS. Hence, also provided is the use of PTX3 and PCT, or a fragment thereof, as a biomarker for the diagnosis of whether a subject presenting with one or more signs of SIRS has severe sepsis. A further preferred embodiment provides a method for the diagnosis of whether a subject presenting with one or more signs of SIRS has severe sepsis, wherein the method comprises measuring the quantity of PTX3 and PCT, or a fragment thereof, in a sample from the subject.

The inventors also interestingly found that combining evaluation of IL1R2 with the evaluation of PCT greatly improves the performance of IL1R2 to diagnose severe sepsis in subjects presenting with one or more signs of SIRS. Hence, also provided is the use of IL1R2 and PCT, or a fragment thereof, as a biomarker for the diagnosis of whether a subject presenting with one or more signs of SIRS has severe sepsis. A further preferred embodiment provides a method for the diagnosis of whether a subject presenting with one or more signs of SIRS has severe sepsis, wherein the method comprises measuring the quantity of IL1R2 and PCT, or a fragment thereof, in a sample from the subject.

In certain embodiments, the present uses and methods may evaluate a single variable, such as a single biomarker. In other embodiments, the present uses and methods may evaluate two or more variables, such as one biomarker and one or more clinical parameters, or two or more biomarkers and optionally one or more clinical parameters. Each so-measured biomarker and/or clinical parameter may be evaluated separately and independently, or one may generate a profile from the values or quantities for two or more variables. It shall thus also be understood by the skilled man that any value or quantity as referred to herein may also encompass a profile. Similarly, any reference value as referred to herein may also encompass a reference profile.

The inventors further found in exemplary studies that combining evaluation of Procalcitonin (PCT) with the evaluation of one or more markers selected from the group consisting of PRTN3, GSHB, PTX3, VCAM1, PSA3, NID1, GOLM1, ATF6A, ICAM1, PIGR, CALU, EXT2, PHLD, FGL1, and IL1R2, in a subject, greatly improves the performance of PCT to diagnose sepsis, in particular to diagnose whether a subject presenting with one or more signs of SIRS has infection-free SIRS or has sepsis.

Hence, a further aspect relates to a test panel for the diagnosis, prediction, prognosis, and/or monitoring of a systemic inflammatory condition in a subject, preferably wherein the systemic inflammatory condition is sepsis or SIRS, more preferably wherein the systemic inflammatory condition is sepsis, the test panel comprising or consisting of: measurement of the level of PCT or a fragment thereof in the subject; and measurement of the level of one or more (such as, e.g., one, two, three, four or five; such as, preferably, one, two, three or four; such as, more preferably, one, two or three; such as, even more preferably, one or two) markers selected from the group consisting of PRTN3, GSHB, PTX3, VCAM1, PSA3, NID1, GOLM1, ATF6A, ICAM1, PIGR, CALU, EXT2, PHLD, FGL1, IL1R2, CATG, and ELNE, or a fragment thereof, or selected from the group consisting of PRTN3, GSHB, PTX3, VCAM1, PSA3, NID1, GOLM1, ATF6A, ICAM1, PIGR, CALU, EXT2, PHLD, FGL1, and IL1R2, or a fragment thereof, in the subject.

Certain embodiments provide a test panel for the diagnosis, prediction, prognosis, and/or monitoring of a systemic inflammatory condition in a subject, preferably wherein the systemic inflammatory condition is sepsis or SIRS, more preferably wherein the systemic inflammatory condition is sepsis, the test panel comprising or consisting of: measurement of the level of PCT or a fragment thereof in the subject; and measurement of the level of at least two, such as exactly two, markers selected from the group consisting of PRTN3, GSHB, PTX3, VCAM1, PSA3, NID1, GOLM1, ATF6A, ICAM1, PIGR, CALU, EXT2, PHLD, FGL1, ILIR2, CATG, and ELNE, or a fragment thereof, or selected from the group consisting of PRTN3, GSHB, PTX3, VCAM1, PSA3, NID1, GOLM1, ATF6A, ICAM1, PIGR, CALU, EXT2, PHLD, FGL1, and ILIR2, or a fragment thereof, in the subject.

In certain embodiments of the present test panels, measurement of the level of PTX3 or a fragment thereof in the subject may be included in the panel instead of or in addition to, preferably instead of, measurement of the level of PCT or a fragment thereof. This may particularly apply to test panels which comprise at least one or preferably both of measurement of the level of PRTN3 or a fragment thereof and measurement of the level of GSHB or a fragment thereof, e.g., an exemplary but non-limiting panel comprising or consisting of PTX3, PRTN3 and GSHB.

In certain embodiments, any one of the present test panels may further comprise the measurement of white blood cell (WBC) count in the subject, e.g., an exemplary but non-limiting panel comprising or consisting of PCT, GSHB and WBC.

Any test panel disclosed in the present specification may in certain preferred embodiments comprise or consist of two or more of the aforementioned constituents, for example of two, three, four, five or six constituents, in other preferred examples of two, three, four or five constituents, in yet further preferred examples of two, three or four constituents, and particularly preferably of two or three of the aforementioned constituents.

In certain experiments, the inventors realised that test panels comprising or consisting of i) measurement of the level of PCT or a fragment thereof or measurement of the level of PTX3 or a fragment thereof in the subject and ii) at least one and preferably both of measurement of the level of PRTN3 or a fragment thereof and measurement of the level of GSHB or a fragment thereof in the subject, showed advantageously improved performance diagnosis of sepsis, in particular for discriminating infection-free SIRS from sepsis compared with PCT as a standard single marker.

Hence, in certain embodiments, the test panel as taught herein may comprise or consist of: measurement of the level of PCT or PTX3, or a fragment thereof, in the subject, and at least one and preferably both of measurement of the level of PRTN3 or a fragment thereof and measurement of the level of GSHB or a fragment thereof in the subject. In certain embodiments, the measurement of the level of PRTN3 or a fragment thereof may be replaced or complemented by one or both of the measurement of the level of CATG or a fragment thereof and the measurement of the level of ELNE or a fragment thereof in the subject.

In certain embodiments, the test panels as taught herein may advantageously include the assessment (i.e., measurement of the presence or absence and/or quantity) of one or more other biomarkers or clinical parameters relevant for the diagnosis, prediction, prognosis and/or monitoring of the herein taught diseases or conditions, in particular systemic inflammatory conditions, more particularly SIRS or sepsis. By means of example and not limitation, such other biomarkers include C-reactive protein (CRP), lactate, Cystatin C (CYTC), Neutrophil gelatinase-associated lipocalin (NGAL) and interleukin-6 (IL6), or a fragment thereof. Preferably, the other biomarker may be IL6. By means of example and not limitation, such clinical parameters may be white blood cell (WBC) count, kidney function parameters such as serum creatinine and/or urine output, respiratory system function such as PaO2/FiO2, nervous system function preferably expressed as Glasgow coma scale, cardiovascular function preferably expressed as mean arterial pressure, liver function preferably expressed as bilirubin concentration, coagulation function preferably expressed as platelet concentration, etc. Preferably, the clinical parameter may be white blood cell (WBC) count.

In preferred embodiments, the test panel may comprise or consist of: measurement of the level of PCT or a fragment thereof and measurement of the level of PRTN3 or a fragment thereof; or measurement of the level of PCT or a fragment thereof and measurement of the level of GSHB or a fragment thereof; or measurement of the level of PCT or a fragment thereof, measurement of the level of PRTN3 or a fragment thereof, and measurement of the level of GSHB or a fragment thereof; or measurement of the level of PTX3 or a fragment thereof, measurement of the level of PRTN3 or a fragment thereof, and measurement of the level of GSHB or a fragment thereof. In certain embodiments, the measurement of the level of PRTN3 or a fragment thereof may be replaced or complemented by one or both of the measurement of the level of CATG or a fragment thereof and the measurement of the level of ELNE or a fragment thereof in the subject.

In further preferred embodiments, the test panel may comprise or consist of: measurement of the level of PCT or a fragment thereof, measurement of the level of PRTN3 or a fragment thereof, and measurement of the level of VCAM1 or a fragment thereof; or measurement of the level of PCT or a fragment thereof, measurement of the level of PRTN3 or a fragment thereof, and measurement of the level of PSA3 or a fragment thereof; or measurement of the level of PCT or a fragment thereof, measurement of the level of PRTN3 or a fragment thereof, and measurement of the level of NID1 or a fragment thereof; or measurement of the level of PCT or a fragment thereof, measurement of the level of PRTN3 or a fragment thereof, and measurement of the level of GOLM1 or a fragment thereof; or measurement of the level of PCT or a fragment thereof, measurement of the level of PRTN3 or a fragment thereof, and measurement of the level of PTX3 or a fragment thereof; or measurement of the level of PCT or a fragment thereof, measurement of the level of GSHB or a fragment thereof, and measurement of the level of ATF6A or a fragment thereof; or measurement of the level of PCT or a fragment thereof, measurement of the level of GSHB or a fragment thereof, and measurement of the level of ICAM1 or a fragment thereof; or measurement of the level of PCT or a fragment thereof, measurement of the level of GSHB or a fragment thereof, and measurement of WBC; or measurement of the level of PCT or a fragment thereof, measurement of the level of GSHB or a fragment thereof, and measurement of the level of PIGR or a fragment thereof; or measurement of the level of PCT or a fragment thereof, measurement of the level of GSHB or a fragment thereof, and measurement of the level of PTX3 or a fragment thereof; or measurement of the level of PCT or a fragment thereof, measurement of the level of GSHB or a fragment thereof, and measurement of the level of CALU or a fragment thereof; or measurement of the level of PCT or a fragment thereof, measurement of the level of VCAM1 or a fragment thereof, and measurement of the level of IL6 or a fragment thereof; or measurement of the level of PCT or a fragment thereof, measurement of the level of VCAM1 or a fragment thereof, and measurement of the level of EXT2 or a fragment thereof; or measurement of the level of PCT or a fragment thereof, measurement of the level of PHLD or a fragment thereof, and measurement of the level of EXT2 or a fragment thereof; or measurement of the level of PCT or a fragment thereof, measurement of the level of FGL1 or a fragment thereof, and measurement of the level of GOLM1 or a fragment thereof; or measurement of the level of PCT or a fragment thereof, measurement of the level of FGL1 or a fragment thereof, and measurement of the level of NID1 or a fragment thereof. In certain embodiments, the measurement of the level of PRTN3 or a fragment thereof may be replaced or complemented by one or both of the measurement of the level of CATG or a fragment thereof and the measurement of the level of ELNE or a fragment thereof in the subject.

Exemplary, non-limiting test panels embodying the principles of the invention include those individualised in Tables 14 and 15, as well as test panels as defined herein which comprise those individualised in Tables 14 and 15.

In a further aspect, the invention relates to the use of any one of the test panels as defined herein for the diagnosis, prediction, prognosis and/or monitoring of a systemic inflammatory condition in a subject, preferably wherein the systemic inflammatory condition is sepsis or SIRS, more preferably wherein the systemic inflammatory condition is sepsis. The present uses may be adequately qualified as in vitro or ex vivo uses, in that they apply particular in vitro or ex vivo processing and analysis on a sample obtained from a subject.

In certain particularly preferred embodiments, the use of any one of the test panels as defined herein may be for the diagnosis of sepsis, particularly for diagnosis whether a subject presenting with one or more signs of systemic inflammatory response syndrome (SIRS) has infection-free SIRS or has sepsis.

In certain other embodiments, the use of any one of the test panels as defined herein may be for the diagnosis, prediction and/or prognosis of the severity of the systemic inflammatory condition, preferably wherein the systemic inflammatory condition is SIRS or sepsis, in a subject. More preferably, said diagnosis, prediction and/or prognosis of the severity of the systemic inflammatory condition in the subject comprises the prediction of mortality in the subject or the prognosis that the systemic inflammatory condition will result in death of the subject, or comprises the diagnosis, prediction and/or prognosis of organ failure or multi-organ dysfunction syndrome in the subject.

In certain further embodiments, the use of any one of the test panels as defined herein is for monitoring of a systemic inflammatory condition in a subject, preferably in the course of a medical treatment of the subject, preferably wherein the systemic inflammatory condition is sepsis or SIRS, more preferably wherein the systemic inflammatory condition is sepsis.

In another aspect, the invention relates to a method for the diagnosis, prediction, prognosis and/or monitoring of a systemic inflammatory condition in a subject, wherein the method comprises testing or evaluating in the subject any one of the test panels as defined herein. In some preferred embodiments, said systemic inflammatory condition may be sepsis. In other embodiments, said systemic inflammatory condition may be SIRS. The present methods may be adequately qualified as in vitro or ex vivo methods, in that they apply particular in vitro or ex vivo processing and analysis steps on a sample obtained from a subject.

In certain embodiments, the method for the diagnosis, prediction and/or prognosis of a systemic inflammatory condition in a subject, preferably wherein the systemic inflammatory condition is sepsis or SIRS, more preferably wherein the systemic inflammatory condition is sepsis, using any one of the test panels as defined herein, may comprise the steps of: (i) measuring the quantity of the biomarkers comprised in said test panel in a sample from the subject and, where the test panel comprises a clinical parameter or parameters, measuring or scoring said clinical parameter or parameters in the subject; (ii) comparing the quantity of the biomarkers as measured in (i) and, where the test panel comprises a clinical parameter or parameters, the measurement or score of said parameter or parameters as measured or scored in (i) with a reference value of the quantity of the biomarkers comprised in the test panel and, where the test panel comprises a clinical parameter or parameters, of measurement or score for said clinical parameter or parameters, said reference value representing a known diagnosis, prediction and/or prognosis of a systemic inflammatory condition; (iii) finding a deviation or no deviation of the quantity of the biomarkers as measured in (i) and, where the test panel comprises a clinical parameter or parameters, of the measurement or score of said parameter or parameters as measured or scored in (i) from the reference value; and (iv) attributing said finding of deviation or no deviation to a particular diagnosis, prediction and/or prognosis of a systemic inflammatory condition in the subject. In some preferred embodiments, said systemic inflammatory condition may be sepsis. In other embodiments, said systemic inflammatory condition may be SIRS.

In a further aspect the invention relates to a system comprising:
a computer data repository that comprises a reference value of the quantity of biomarkers comprised in a test panel as defined herein and, where the test panel comprises a clinical parameter or parameters, of measurement or score for said clinical parameter or parameters, said reference value representing a known diagnosis, prediction and/or prognosis of a systemic inflammatory condition; and
a computer system programmed to access the data repository and to use information from the data repository in combination with information on the quantity of biomarkers comprised in the test panel in a sample from a subject and, where the test panel comprises a clinical parameter or parameters, on measurement or score for said clinical parameter or parameters in the subject, to make a diagnosis, prediction and/or prognosis of a systemic inflammatory condition in the subject. In some preferred embodiments, said systemic inflammatory condition may be sepsis. In other embodiments, said systemic inflammatory condition may be SIRS.

Related embodiments of the invention concern a method for making diagnosis, prediction and/or prognosis of a systemic inflammatory condition in a subject comprising:
(i) receiving data representative of values of the quantity of biomarkers comprised in a test panel as defined herein in a sample from a subject and, where the test panel comprises a clinical parameter or parameters, on measurement or score for said clinical parameter or parameters in the subject;
(ii) accessing a data repository on a computer, said data repository comprising a reference value of the quantity of biomarkers comprised in the test panel and, where the test panel comprises a clinical parameter or parameters, of measurement or score for said clinical parameter or parameters, said reference value representing a known diagnosis, prediction and/or prognosis of a systemic inflammatory condition; and
(iii) comparing the data as received in (i) with the reference value in the data repository on the computer, thereby making a diagnosis, prediction and/or prognosis of a systemic inflammatory condition in the subject.

In certain embodiments, the determination of what action is to be taken, e.g., by a clinician, in view of said diagnosis, prediction and/or prognosis is performed by a (the) computer. In certain embodiments, a (the) computer reports (i.e., generates an electronic report of) the action to be taken, preferably substantially in real time.

In certain embodiments, the method for monitoring the systemic inflammatory condition, preferably in the course of a medical treatment of the subject, using any one of the test panels as defined herein, may comprise the steps of: (i) measuring the quantity of the biomarkers comprised in said test panel in a sample from a subject and, where the test panel comprises a clinical parameter or parameters, measuring or scoring said clinical parameter or parameters in the subject, at two or more successive time points; (ii) comparing the quantity of the biomarkers as measured in (i) and, where the test panel comprises a clinical parameter or parameters, the measurement or score of said parameter or parameters as measured or scored in (i) between said two or more successive time points; (iii) finding a deviation or no deviation of the quantity of the biomarkers as measured in (i) and, where the test panel comprises a clinical parameter or parameters, of the measurement or score of said parameter or parameters as measured or scored in (i) between said two or more successive time points; and (iv) attributing said finding of deviation or no deviation to a change in the systemic inflammatory condition in the subject between the two or more successive time points. Such method thus allows the monitoring of the systemic inflammatory condition in a subject over time. In some preferred embodiments, said systemic inflammatory condition may be sepsis. In other embodiments, said systemic inflammatory condition may be SIRS.

In certain particularly preferred embodiments, the method using any one of the test panels as defined herein may be for the diagnosis of whether a subject presenting with one or more signs of systemic inflammatory response syndrome (SIRS) has infection-free SIRS or has sepsis.

In certain other embodiments, the method using any one of the test panels as defined herein may be for the diagnosis, prediction and/or prognosis of the severity of the systemic inflammatory condition, preferably wherein the systemic inflammatory condition is SIRS or sepsis, in a subject. More preferably, said diagnosis, prediction and/or prognosis of the severity of the systemic inflammatory condition in the subject comprises the prediction of mortality in the subject or the prognosis that the systemic inflammatory condition will result in death of the subject, or comprises the diagnosis, prediction and/or prognosis of organ failure or multi-organ dysfunction syndrome in the subject.

In certain further embodiments, the method using any one of the test panels as defined herein is for monitoring of a systemic inflammatory condition in a subject, preferably in the course of a medical treatment of the subject, preferably wherein the systemic inflammatory condition is sepsis or SIRS, more preferably wherein the systemic inflammatory condition is sepsis.

The above methods for the diagnosis, prediction, prognosis and/or monitoring of the systemic inflammatory condition in the subject may in certain embodiments also be applied to determine whether the subject is or is not—for example, still is, or is no longer—in need of a therapeutic or prophylactic (preventative) treatment of the systemic inflammatory condition. For example, a treatment may be particularly indicated where the methods allow for a conclusion that the subject has or is at risk of having the systemic inflammatory condition, or has a poor prognosis for the systemic inflammatory condition, such as for example organ failure, multiple organ dysfunction syndrome (MODS) or death, or displays a detrimental development of the systemic inflammatory condition. Without limitation, a patient with the systemic inflammatory condition upon admission to or during stay in a medical care centre such as ICU may be tested as taught herein for the necessity of continuing the treatment of said systemic inflammatory condition, and may be discharged when such treatment is no longer needed or is needed only to a given limited extent.

In certain embodiments, the invention relates to a method for treating a systemic inflammatory condition in a subject in need of said treatment, the method comprising the steps of:

(i) measuring the quantity of the biomarkers comprised in a test panel as defined herein in a sample from the subject and, where the test panel comprises a clinical parameter or parameters, measuring or scoring said clinical parameter or parameters in the subject;
(ii) comparing the quantity of the biomarkers as measured in (i) and, where the test panel comprises a clinical parameter or parameters, the measurement or score of said parameter or parameters as measured or scored in (i) with a reference value of the quantity of the biomarkers comprised in the test panel and, where the test panel comprises a clinical parameter or parameters, of measurement or score for said clinical parameter or parameters, said reference value representing a known diagnosis, prediction and/or prognosis of a systemic inflammatory condition;
(iii) finding a deviation or no deviation of the quantity of the biomarkers as measured in (i) and, where the test panel comprises a clinical parameter or parameters, of the measurement or score of said parameter or parameters as measured or scored in (i) from the reference value;
(iv) attributing said finding of deviation or no deviation to a particular diagnosis, prediction and/or prognosis of a systemic inflammatory condition in the subject;
(v) inferring from said particular diagnosis, prediction and/or prognosis of a systemic inflammatory condition in the subject the presence or absence of a need for a therapeutic or prophylactic treatment of the systemic inflammatory condition in the subject; and
(vi) administering a therapeutically or prophylactically effective amount of an active pharmaceutical ingredient capable of treating the systemic inflammatory condition to said subject when the subject is in need of said treatment.

In some preferred embodiments, said systemic inflammatory condition may be sepsis. In other embodiments, said systemic inflammatory condition may be SIRS. Examples of active pharmaceutical ingredients capable of treating systemic inflammatory conditions may include, without limitation, anti-microbial agents, preferably anti-bacterial agents, such as antibiotics; analgesics; antipyretics; and anti-inflammatory drugs, such as non-steroidal anti-inflammatory drugs (NSAID). Any one or a combination of two or more may be used.

Any one prediction, diagnosis, prognosis and/or monitoring use or method as taught herein may preferably allow for sensitivity and/or specificity (preferably, sensitivity and specificity) of at least 50%, at least 60%, at least 70% or at least 80%, e.g., ≧85% or ≧90% or ≧95%, e.g., between about 80% and 100% or between about 85% and 95%.

Reference throughout this specification to “diseases”, “conditions” or a similar reference encompasses any such diseases and conditions as disclosed herein insofar consistent with the context of a particular recitation. More specifically, such disease and conditions encompass systemic inflammatory conditions, including SIRS and sepsis, as well as any aspects or clinical outcomes relevant in the context of said diseases and conditions.

The uses and methods for the diagnosis, prediction, prognosis and/or monitoring of the diseases and conditions taught herein may be used in subjects who have not yet been diagnosed as having such (for example, preventative screening), or who have been diagnosed as having such, or who are suspected of having such (for example, display one or more characteristic signs and/or symptoms), or who are at risk of developing such (for example, genetic predisposition; presence of one or more developmental, environmental or behavioural risk factors). The uses and methods may also be used to detect various stages of progression or severity of the diseases and conditions. The uses and methods may also be used to detect response of the diseases and conditions to prophylactic or therapeutic treatments or other interventions. The uses and methods may furthermore be used to help the medical practitioner in deciding upon worsening, status-quo, partial recovery, or complete recovery of the subject from the diseases and conditions, resulting in either further treatment or observation or in discharge of the patient from a medical care centre.

Reference values as employed herein may be established according to known procedures previously employed for other biomarkers. Such reference values may be established either within (i.e., constituting a step of) or external to (i.e., not constituting a step of) any one of the methods as taught herein. Accordingly, any one of the methods taught herein may comprise a step of establishing a reference value for the quantity of one or more markers as taught herein, said reference value representing either (a) a prediction or diagnosis of the absence of the diseases or conditions as taught herein or a good prognosis thereof, or (b) a prediction or diagnosis of the diseases or conditions as taught herein or a poor prognosis thereof.

A further aspect thus provides a method for establishing a reference value for the quantity of any one or more markers selected from the group consisting of PRTN3, MRC1, EXT2, IL1R2, PTX3, MA1A1, ACBP, LMAN2, ACHA7, ATF6A, B4GT1, CAMP, GOLM1, NID1, MMP3, LBP, FBLN1, PIGR, TIMP1, PHLD, ANGT, CBPN, CH3L1, CSF1, DAG1, FBN1, FGL1, GSHB, ICAM1, LUM, S10A9, SAA, SRGN, VCAM1, CALU, EMAL3, GDIR2, GUC2B, HSP7C, I13R1, MOES, PDIA6, PSA3, PTPRG, S10A8, CATG, and ELNE, or selected from the group consisting of PRTN3, MRC1, EXT2, IL1R2, PTX3, MA1A1, ACBP, LMAN2, ACHA7, ATF6A, B4GT1, CAMP, GOLM1, NID1, MMP3, LBP, FBLN1, PIGR, TIMP1, PHLD, ANGT, CBPN, CH3L1, CSF1, DAG1, FBN1, FGL1, GSHB, ICAM1, LUM, S10A9, SAA, SRGN, VCAM1, CALU, EMAL3, GDIR2, GUC2B, HSP7C, I13R1, MOES, PDIA6, PSA3, PTPRG, and S10A8, said reference value representing:

(a) a prediction or diagnosis of the absence of the diseases or conditions as taught herein or a good prognosis thereof, or
(b) a prediction or diagnosis of the diseases or conditions as taught herein or a poor prognosis thereof, comprising:
(i) measuring the quantity of said one or more markers in:
(i a) one or more samples from one or more subjects not having the respective diseases or conditions or not being at risk of having such or having a good prognosis for such, or
(i b) one or more samples from one or more subjects having the respective diseases or conditions or being at risk of having such or having a poor prognosis for such, and
   (ii) storing the quantity of said one or more markers:
(ii a) as measured in (i a) as the reference value representing the prediction or diagnosis of the absence of the respective diseases or conditions or representing the good prognosis therefore, or
(ii b) as measured in (i b) as the reference value representing the prediction or diagnosis of the respective diseases or conditions or representing the poor prognosis therefore.

The present methods may otherwise employ reference profiles for the quantity of any one, two or more markers selected from the group consisting of PRTN3, MRC1, EXT2, IL1R2, PTX3, MA1A1, ACBP, LMAN2, ACHA7, ATF6A, B4GT1, CAMP, GOLM1, NID1, MMP3, LBP, FBLN1, PIGR, TIMP1, PHLD, ANGT, CBPN, CH3L1, CSF1, DAG1, FBN1, FGL1, GSHB, ICAM1, LUM, S10A9, SAA, SRGN, VCAM1, CALU, EMAL3, GDIR2, GUC2B, HSP7C, I13R1, MOES, PDIA6, PSA3, PTPRG, S10A8, CATG, and ELNE, or selected from the group consisting of PRTN3, MRC1, EXT2, IL1R2, PTX3, MA1A1, ACBP, LMAN2, ACHA7, ATF6A, B4GT1, CAMP, GOLM1, NID1, MMP3, LBP, FBLN1, PIGR, TIMP1, PHLD, ANGT, CBPN, CH3L1, CSF1, DAG1, FBN1, FGL1, GSHB, ICAM1, LUM, S10A9, SAA, SRGN, VCAM1, CALU, EMAL3, GDIR2, GUC2B, HSP7C, I13R1, MOES, PDIA6, PSA3, PTPRG, and S10A8, and optionally the presence or absence and/or quantity of one or more other biomarkers, which may be established according to known procedures previously employed for other biomarkers. Such reference profiles may be established either within (i.e., constituting a step of) or external to (i.e., not constituting a step of) the present methods. Accordingly, the methods taught herein may comprise a step of establishing a reference profile for the quantity of any one, any two or more markers as taught herein and optionally the presence or absence and/or quantity of one or more other biomarkers, said reference profile representing either (a) a prediction or diagnosis of the absence of the diseases or conditions as taught herein or a good prognosis therefore, or (b) a prediction or diagnosis of the diseases or conditions as taught herein or a poor prognosis therefore.

A further aspect provides a method for establishing a reference profile for the quantity of any one, two or more markers selected from the group consisting of PRTN3, MRC1, EXT2, IL1R2, PTX3, MA1A1, ACBP, LMAN2, ACHA7, ATF6A, B4GT1, CAMP, GOLM1, NID1, MMP3, LBP, FBLN1, PIGR, TIMP1, PHLD, ANGT, CBPN, CH3L1, CSF1, DAG1, FBN1, FGL1, GSHB, ICAM1, LUM, S10A9, SAA, SRGN, VCAM1, CALU, EMAL3, GDIR2, GUC2B, HSP7C, I13R1, MOES, PDIA6, PSA3, PTPRG, S10A8, CATG, and ELNE, or selected from the group consisting of PRTN3, MRC1, EXT2, IL1R2, PTX3, MA1A1, ACBP, LMAN2, ACHA7, ATF6A, B4GT1, CAMP, GOLM1, NID1, MMP3, LBP, FBLN1, PIGR, TIMP1, PHLD, ANGT, CBPN, CH3L1, CSF1, DAG1, FBN1, FGL1, GSHB, ICAM1, LUM, S10A9, SAA, SRGN, VCAM1, CALU, EMAL3, GDIR2, GUC2B, HSP7C, I13R1, MOES, PDIA6, PSA3, PTPRG and S10A8, and optionally the presence or absence and/or quantity of one or more other biomarkers and/or clinical parameters useful for the diagnosis, prediction, prognosis and/or monitoring of the diseases or conditions as taught herein, said reference profile representing:

(a) a prediction or diagnosis of the absence of the respective diseases or conditions or a good prognosis therefore, or
(b) a prediction or diagnosis of the respective diseases or conditions or a poor prognosis therefore,
comprising:
(i) determining two or more variables comprising measuring the quantity of one or more markers as taught herein and optionally the presence or absence and/or quantity of said one or more other biomarkers or clinical parameters in:
(i a) one or more samples from one or more subjects not having the respective diseases or conditions or not being at risk of having such or having a good prognosis for such; or
(i b) one or more samples from one or more subjects having the respective diseases or conditions or being at risk of having such or having a poor prognosis for such;
   (ii)
(ii a) using the measurements of (i a) to create a profile of said two or more variables; or
(ii b) using the measurements of (i b) to create a profile of said two or more variables;
   (iii)
(iii a) storing the profile of (ii a) as the reference profile representing the prediction or diagnosis of the absence of the respective diseases or conditions or representing the good prognosis therefore; or
(iii b) storing the profile of (ii b) as the reference profile representing the prediction or diagnosis of the respective diseases conditions or representing the poor prognosis therefore.

Further provided is a method for establishing a base-line value in a subject, comprising: (i) measuring the quantity of any one or more markers selected from the group consisting of PRTN3, MRC1, EXT2, IL1R2, PTX3, MA1A1, ACBP, LMAN2, ACHA7, ATF6A, B4GT1, CAMP, GOLM1, NID1, MMP3, LBP, FBLN1, PIGR, TIMP1, PHLD, ANGT, CBPN, CH3L1, CSF1, DAG1, FBN1, FGL1, GSHB, ICAM1, LUM, S10A9, SAA, SRGN, VCAM1, CALU, EMAL3, GDIR2, GUC2B, HSP7C, I13R1, MOES, PDIA6, PSA3, PTPRG, S10A8, CATG, and ELNE, or a fragment thereof, or selected from the group consisting of PRTN3, MRC1, EXT2, ILIR2, PTX3, MA1A1, ACBP, LMAN2, ACHA7, ATF6A, B4GT1, CAMP, GOLM1, NID1, MMP3, LBP, FBLN1, PIGR, TIMP1, PHLD, ANGT, CBPN, CH3L1, CSF1, DAG1, FBN1, FGL1, GSHB, ICAM1, LUM, S10A9, SAA, SRGN, VCAM1, CALU, EMAL3, GDIR2, GUC2B, HSP7C, I13R1, MOES, PDIA6, PSA3, PTPRG, and S10A8, or a fragment thereof, in the sample from the subject at two or more time points when the subject does not have the diseases or conditions as taught herein, and (ii) calculating the range or mean value (where necessary with appropriate statistics) of the quantities measured in (i), whereby the range or mean value calculated in (ii) represents the base-line for said subject. In some instances, such base-line value may be employed as reference value in the context of the present uses and methods.

The respective quantities, measurements or scores for the biomarker(s) and parameter(s) in the present test panels may be evaluated separately and individually, i.e., each compared with its corresponding reference value. More advantageously, the quantities, measurements or scores for the biomarker(s) and parameter(s) may be used to establish a biomarker-and-parameter profile, which can be suitably compared with a corresponding multi-parameter reference value. In yet another alternative, the quantities, measurements or scores for the biomarker(s) and parameter(s) may each be modulated by an appropriate weighing factor and added up to yield a single value, which can then be suitably compared with a corresponding reference value obtained accordingly. One shall appreciate that such weighing factors may depend on the methodology used to quantify biomarkers and measure or score parameters, and for each particular experimental setting may be determined and comprised in a model suitable for diagnosis, prediction and/or prognosis of the diseases and conditions as taught herein. Various methods can be used for the purpose of establishing such models, e.g., support vector machine, Bayes classifiers, logistic regression, etc. (Cruz et al. Applications of Machine Learning in Cancer Prediction and Prognosis. Cancer Informatics 2007; 2; 59-77).

Reference values as employed herein may be established according to known procedures previously employed for other test panels comprising biomarkers and/or clinical parameters. Reference values may be established either within (i.e., constituting a step of) or external to (i.e., not constituting a step of) the methods and uses as taught herein. Accordingly, any one of the methods or uses taught herein may comprise a step of establishing a requisite reference value.

Hence, also provided is a method for establishing a reference value for a test panel as taught herein, said reference value representing:

(a) a prediction or diagnosis of the absence of the diseases or conditions as taught herein or a good prognosis thereof, or
(b) a prediction or diagnosis of the diseases or conditions as taught herein or a poor prognosis thereof, comprising:
(i) measuring the quantity of the biomarker or biomarkers comprised in said test panel in a sample from, and, where the test panel comprises a clinical parameter or parameters, measuring or scoring the parameter or parameters comprised in said test panel in:
(i a) one or more subjects not having the respective diseases or conditions or not being at risk of having such or having a good prognosis for such, or
(i b) one or more subjects having the respective diseases or conditions or being at risk of having such or having a poor prognosis for such, and
(ii a) establishing from the quantity of the biomarker or biomarkers and, where the test panel comprises a clinical parameter or parameters, measurement or score of the parameter or parameters as measured in (i a) the reference value representing the prediction or diagnosis of the absence of the respective diseases or conditions or representing the good prognosis therefore, or
(ii b) establishing from the quantity of the biomarker or biomarkers and, where the test panel comprises a clinical parameter or parameters, measurement or score of the parameter or parameters as measured in (i b) the reference value representing the prediction or diagnosis of the respective diseases or conditions or representing the poor prognosis therefore.

Further provided is a method for establishing a base-line reference value for a test panel as taught herein in a subject, comprising: (i) measuring the quantity of the biomarker or biomarkers comprised in said test panel in a sample from the subject, and, where the test panel comprises a clinical parameter or parameters, measuring or scoring the parameter or parameters comprised in said test panel in the subject at one or more time points when the subject is not suffering from the diseases or conditions as taught herein, and (ii) establishing from the quantity of the biomarker or biomarkers and, where the test panel comprises a clinical parameter or parameters, measurement or score of the parameter or parameters as measured in (i) a range or mean reference value for the subject, which is the base-line reference value for said subject.

The quantity of any one or more markers as taught herein, including also markers assayed in the context of test panels, preferably of any one or more markers selected from the group consisting of PRTN3, MRC1, EXT2, IL1R2, PTX3, MA1A1, ACBP, LMAN2, ACHA7, ATF6A, B4GT1, CAMP, GOLM1, NID1, MMP3, LBP, FBLN1, PIGR, TIMP1, PHLD, ANGT, CBPN, CH3L1, CSF1, DAG1, FBN1, FGL1, GSHB, ICAM1, LUM, S10A9, SAA, SRGN, VCAM1, CALU, EMAL3, GDIR2, GUC2B, HSP7C, I13R1, MOES, PDIA6, PSA3, PTPRG, S10A8, CATG, and ELNE, or a fragment thereof, or selected from the group consisting of PRTN3, MRC1, EXT2, ILIR2, PTX3, MA1A1, ACBP, LMAN2, ACHA7, ATF6A, B4GT1, CAMP, GOLM1, NID1, MMP3, LBP, FBLN1, PIGR, TIMP1, PHLD, ANGT, CBPN, CH3L1, CSF1, DAG1, FBN1, FGL1, GSHB, ICAM1, LUM, S10A9, SAA, SRGN, VCAM1, CALU, EMAL3, GDIR2, GUC2B, HSP7C, I13R1, MOES, PDIA6, PSA3, PTPRG, and S10A8, or a fragment thereof, and/or the presence or absence and/or quantity of the one or more other biomarkers (e.g., PCT or IL6), may be measured by any suitable technique such as may be known in the art.

For example, one may employ binding agents capable of specifically binding to the respective biomarkers and/or to fragments thereof. Binding agent may be inter alia an antibody, aptamer, photoaptamer, protein, peptide, peptidomimetic or a small molecule. For instance, one may employ an immunoassay technology or a mass spectrometry analysis method or a chromatography method, or RNA analysis tools such as northern blotting, or (quantitative) RT-PCR, or a combination of said methods.

Accordingly, further disclosed herein are the methods as taught herein, wherein the quantity of said one or more markers selected from the group consisting of PRTN3, MRC1, EXT2, IL1R2, PTX3, MA1A1, ACBP, LMAN2, ACHA7, ATF6A, B4GT1, CAMP, GOLM1, NID1, MMP3, LBP, FBLN1, PIGR, TIMP1, PHLD, ANGT, CBPN, CH3L1, CSF1, DAG1, FBN1, FGL1, GSHB, ICAM1, LUM, S10A9, SAA, SRGN, VCAM1, CALU, EMAL3, GDIR2, GUC2B, HSP7C, I13R1, MOES, PDIA6, PSA3, PTPRG, S10A8, CATG, and ELNE, or a fragment thereof, or selected from the group consisting of PRTN3, MRC1, EXT2, ILIR2, PTX3, MA1A1, ACBP, LMAN2, ACHA7, ATF6A, B4GT1, CAMP, GOLM1, NID1, MMP3, LBP, FBLN1, PIGR, TIMP1, PHLD, ANGT, CBPN, CH3L1, CSF1, DAG1, FBN1, FGL1, GSHB, ICAM1, LUM, S10A9, SAA, SRGN, VCAM1, CALU, EMAL3, GDIR2, GUC2B, HSP7C, I13R1, MOES, PDIA6, PSA3, PTPRG, and S10A8, or a fragment thereof, and/or the presence or absence and/or quantity of the one or more other biomarkers (e.g., PCT or IL6), is measured using a binding agent capable of specifically binding to the respective markers, using an immunoassay technology, using a mass spectrometry analysis method, using a chromatography method, using RNA analysis tools such as northern blotting, or (quantitative) RT-PCR, or using a combination of said methods, preferably using an immunoassay technology, using a mass spectrometry analysis method, using a chromatography method, or using a combination of said methods.

In preferred embodiments of the methods as taught herein, the quantity of any one or more markers as taught herein, including also markers assayed in the context of test panels, preferably of any one or more markers selected from the group consisting of PRTN3, MRC1, EXT2, ILIR2, PTX3, MA1A1, ACBP, LMAN2, ACHA7, ATF6A, B4GT1, CAMP, GOLM1, NID1, MMP3, LBP, FBLN1, PIGR, TIMP1, PHLD, ANGT, CBPN, CH3L1, CSF1, DAG1, FBN1, FGL1, GSHB, ICAM1, LUM, S10A9, SAA, SRGN, VCAM1, CALU, EMAL3, GDIR2, GUC2B, HSP7C, I13R1, MOES, PDIA6, PSA3, PTPRG and S10A8, CATG, and ELNE, or a fragment thereof, or selected from the group consisting of PRTN3, MRC1, EXT2, IL1R2, PTX3, MA1A1, ACBP, LMAN2, ACHA7, ATF6A, B4GT1, CAMP, GOLM1, NID1, MMP3, LBP, FBLN1, PIGR, TIMP1, PHLD, ANGT, CBPN, CH3L1, CSF1, DAG1, FBN1, FGL1, GSHB, ICAM1, LUM, S10A9, SAA, SRGN, VCAM1, CALU, EMAL3, GDIR2, GUC2B, HSP7C, I13R1, MOES, PDIA6, PSA3, PTPRG, and S10A8, or a fragment thereof, and/or the presence or absence and/or quantity of the one or more other biomarkers (e.g., PCT or IL6), is measured using an immunoassay technology, in preferred but non-limiting examples, using enzyme-linked immunosorbent assay (ELISA), radioimmunoassay (RIA), or ELISPOT technologies, preferably using ELISA.

In preferred embodiments of the methods as taught herein, the quantity of any one or more markers as taught herein, including also markers assayed in the context of test panels, preferably of any one or more markers selected from the group consisting of PRTN3, MRC1, EXT2, IL1R2, PTX3, MA1A1, ACBP, LMAN2, ACHA7, ATF6A, B4GT1, CAMP, GOLM1, NID1, MMP3, LBP, FBLN1, PIGR, TIMP1, PHLD, ANGT, CBPN, CH3L1, CSF1, DAG1, FBN1, FGL1, GSHB, ICAM1, LUM, S10A9, SAA, SRGN, VCAM1, CALU, EMAL3, GDIR2, GUC2B, HSP7C, I13R1, MOES, PDIA6, PSA3, PTPRG and S10A8, CATG, and ELNE, or a fragment thereof, or selected from the group consisting of PRTN3, MRC1, EXT2, IL1R2, PTX3, MA1A1, ACBP, LMAN2, ACHA7, ATF6A, B4GT1, CAMP, GOLM1, NID1, MMP3, LBP, FBLN1, PIGR, TIMP1, PHLD, ANGT, CBPN, CH3L1, CSF1, DAG1, FBN1, FGL1, GSHB, ICAM1, LUM, S10A9, SAA, SRGN, VCAM1, CALU, EMAL3, GDIR2, GUC2B, HSP7C, I13R1, MOES, PDIA6, PSA3, PTPRG, and S10A8, or a fragment thereof, and/or the presence or absence and/or quantity of the one or more other biomarkers (e.g., PCT or IL6), is measured using a binding agent capable of specifically binding to the respective markers, in preferred but non-limiting examples, using an aptamer, antibody, photoaptamer, protein, peptide, peptidomimetic, or a small molecule, preferably using an aptamer or antibody, more preferably using an aptamer.

For example, specific antibodies for PRTN3 are also referred to as Antineutrophil Cytoplasmic Antibodies (ANCA). Such PRTN3 antibodies have been described inter alia by Niles (1996, Annu. Rev. Med., 47:303-13).

Exemplary non-limiting specific antibodies for MRC1 are commercially available, for instance, MRC1 mouse monoclonal antibody with Catalog number 60143-1-Ig from Proteintech Group, Inc. (Chicago, USA), or MRC1 antibodies from LifeSpan Biosciences, Inc. (Seattle, USA) such as MRC1 mouse monoclonal antibody [5C11] LS-B5474 or MRC1 rat monoclonal antibody LS-C124036.

Exemplary non-limiting specific antibodies for PCT are commercially available, for instance, mouse monoclonal antibody LS-C89297 or LS-C89296, or goat polyclonal antibody LS-C41796 from LifeSpan Biosciences, Inc. (Seattle, USA).

Exemplary non-limiting specific antibodies for PTX3 are commercially available, for instance, rabbit polyclonal to Pentraxin 3 with catalogue number ab64860, or mouse monoclonal to Pentraxin 3 with catalogue number ab55641 from Abcam (Cambridge, UK).

Exemplary non-limiting specific antibodies for GSHB are commercially available, for instance, mouse monoclonal GSHB antibody with catalogue number C-5, or rabbit polyclonal GSHB antibody with catalogue number H-300, or goat polyclonal GSHB antibody with catalog number C-15 from Santa Cruz Biotechnology, Inc. (Santa Cruz, USA).

Further disclosed is a kit, in particular for the diagnosis, prediction, prognosis and/or monitoring the diseases or conditions as taught herein in a subject, the kit comprising (i) means for measuring the quantity of any one or more markers selected from the group consisting of PRTN3, MRC1, EXT2, ILIR2, PTX3, MA1A1, ACBP, LMAN2, ACHA7, ATF6A, B4GT1, CAMP, GOLM1, NID1, MMP3, LBP, FBLN1, PIGR, TIMP1, PHLD, ANGT, CBPN, CH3L1, CSF1, DAG1, FBN1, FGL1, GSHB, ICAM1, LUM, S10A9, SAA, SRGN, VCAM1, CALU, EMAL3, GDIR2, GUC2B, HSP7C, I13R1, MOES, PDIA6, PSA3, PTPRG, S10A8, CATG, and ELNE, or a fragment thereof, or selected from the group consisting of PRTN3, MRC1, EXT2, ILIR2, PTX3, MA1A1, ACBP, LMAN2, ACHA7, ATF6A, B4GT1, CAMP, GOLM1, NID1, MMP3, LBP, FBLN1, PIGR, TIMP1, PHLD, ANGT, CBPN, CH3L1, CSF1, DAG1, FBN1, FGL1, GSHB, ICAM1, LUM, S10A9, SAA, SRGN, VCAM1, CALU, EMAL3, GDIR2, GUC2B, HSP7C, I13R1, MOES, PDIA6, PSA3, PTPRG, and S10A8, or a fragment thereof, in a sample from the subject, and optionally and preferably (ii) a reference value of the quantity of said one or more markers or means for establishing said reference value, wherein said reference value represents a known diagnosis, prediction and/or prognosis of the respective diseases or conditions. The kit thus allows one to: measure the quantity of said one or more markers in the sample from the subject by means (i); compare the quantity of said one or more markers measured by means (i) with the reference value of (ii) or established by means (ii); find a deviation or no deviation of the quantity of said one or more markers measured by means (i) from the reference value of (ii); and consequently attribute said finding of deviation or no deviation to a particular diagnosis, prediction and/or prognosis of the diseases or conditions as taught herein in the subject.

The means for measuring the quantity of any one or more markers selected from the group consisting of PRTN3, MRC1, EXT2, IL1R2, PTX3, MA1A1, ACBP, LMAN2, ACHA7, ATF6A, B4GT1, CAMP, GOLM1, NID1, MMP3, LBP, FBLN1, PIGR, TIMP1, PHLD, ANGT, CBPN, CH3L1, CSF1, DAG1, FBN1, FGL1, GSHB, ICAM1, LUM, S10A9, SAA, SRGN, VCAM1, CALU, EMAL3, GDIR2, GUC2B, HSP7C, I13R1, MOES, PDIA6, PSA3, PTPRG, S10A8, CATG, and ELNE, or a fragment thereof, or selected from the group consisting of PRTN3, MRC1, EXT2, ILIR2, PTX3, MA1A1, ACBP, LMAN2, ACHA7, ATF6A, B4GT1, CAMP, GOLM1, NID1, MMP3, LBP, FBLN1, PIGR, TIMP1, PHLD, ANGT, CBPN, CH3L1, CSF1, DAG1, FBN1, FGL1, GSHB, ICAM1, LUM, S10A9, SAA, SRGN, VCAM1, CALU, EMAL3, GDIR2, GUC2B, HSP7C, I13R1, MOES, PDIA6, PSA3, PTPRG, and S10A8, or a fragment thereof, in the present kits may comprise, respectively, one or more binding agents capable of specifically binding to said one or more marker as taught herein or to a fragment thereof. Binding agent may be inter alia an antibody, aptamer, photoaptamer, protein, peptide, peptidomimetic or a small molecule. Preferably, the present kits comprise one or more binding agents capable of specifically binding to said one or more markers as taught herein, such as one or more aptamers, antibodies, photoaptamers, proteins, peptides, peptidomimetics or small molecules, preferably one or more aptamers or antibodies, more preferably one or more aptamers capable of specifically binding to said one or more markers as taught herein. A binding agent may be advantageously immobilised on a solid phase or support. The present kits may employ an immunoassay technology or mass spectrometry analysis technology or chromatography technology, or a combination of said technologies, preferably the present kits employ an immunoassay technology, in preferred but non-limiting examples, enzyme-linked immunosorbent assay (ELISA), radioimmunoassay (RIA), or ELISPOT technologies, preferably using ELISA. Hence, the means for measuring the quantity of marker(s) may be an immunoassay, e.g., an immunoassay employing antibody(ies) and/or aptamers, e.g., ELISA, RIA, or ELISPOT assay.

Disclosed is thus also a kit, particularly a kit for the diagnosis, prediction, prognosis and/or monitoring the diseases or conditions as taught herein comprising: (i) one or more binding agents capable of specifically binding to any one or more markers selected from the group consisting of PRTN3, MRC1, EXT2, IL1R2, PTX3, MA1A1, ACBP, LMAN2, ACHA7, ATF6A, B4GT1, CAMP, GOLM1, NID1, MMP3, LBP, FBLN1, PIGR, TIMP1, PHLD, ANGT, CBPN, CH3L1, CSF1, DAG1, FBN1, FGL1, GSHB, ICAM1, LUM, S10A9, SAA, SRGN, VCAM1, CALU, EMAL3, GDIR2, GUC2B, HSP7C, I13R1, MOES, PDIA6, PSA3, PTPRG, S10A8, CATG, and ELNE, or a fragment thereof, or selected from the group consisting of PRTN3, MRC1, EXT2, ILIR2, PTX3, MA1A1, ACBP, LMAN2, ACHA7, ATF6A, B4GT1, CAMP, GOLM1, NID1, MMP3, LBP, FBLN1, PIGR, TIMP1, PHLD, ANGT, CBPN, CH3L1, CSF1, DAG1, FBN1, FGL1, GSHB, ICAM1, LUM, S10A9, SAA, SRGN, VCAM1, CALU, EMAL3, GDIR2, GUC2B, HSP7C, I13R1, MOES, PDIA6, PSA3, PTPRG, and S10A8, or a fragment thereof; (ii) preferably, a known quantity or concentration of said one or more markers or a fragment thereof (e.g., for use as controls, standards and/or calibrators); (iii) preferably, a reference value of the quantity of said one or more markers or a fragment thereof, or means for establishing said reference value. Said components under (i) and/or (iii) may be suitably labelled as taught elsewhere in this specification.

Further disclosed is the use of any one kit as described herein for the diagnosis, prediction, prognosis and/or monitoring of the respective diseases or conditions in a subject. In particular, disclosed is the use of any one kit as described herein comprising means for measuring the quantity of any one or more markers selected from the group consisting of PRTN3, MRC1, EXT2, ILIR2, PTX3, MA1A1, ACBP, LMAN2, ACHA7, ATF6A, B4GT1, CAMP, GOLM1, NID1, MMP3, LBP, FBLN1, PIGR, TIMP1, PHLD, ANGT, CBPN, CH3L1, CSF1, DAG1, FBN1, FGL1, GSHB, ICAM1, LUM, S10A9, SAA, SRGN, VCAM1, CALU, EMAL3, GDIR2, GUC2B, HSP7C, I13R1, MOES, PDIA6, PSA3, PTPRG, S10A8, CATG, and ELNE, or a fragment thereof, or selected from the group consisting of PRTN3, MRC1, EXT2, IL1R2, PTX3, MA1A1, ACBP, LMAN2, ACHA7, ATF6A, B4GT1, CAMP, GOLM1, NID1, MMP3, LBP, FBLN1, PIGR, TIMP1, PHLD, ANGT, CBPN, CH3L1, CSF1, DAG1, FBN1, FGL1, GSHB, ICAM1, LUM, S10A9, SAA, SRGN, VCAM1, CALU, EMAL3, GDIR2, GUC2B, HSP7C, I13R1, MOES, PDIA6, PSA3, PTPRG, and S10A8, or a fragment thereof, in a sample from a subject, for performing any one of the methods as taught herein. Also intended herein is the use of any one kit as described herein, wherein the kit further comprises a reference value of the quantity of said one or more markers or means for establishing said reference value, wherein said reference value represents a known diagnosis, prediction and/or prognosis of the diseases or conditions as taught herein in the subject.

Further disclosed is a kit, particularly a kit for the diagnosis, prediction, prognosis and/or monitoring of the diseases or conditions as taught herein in a subject, the kit comprising (i) means for measuring the quantity of the biomarker or biomarkers comprised in a test panel as taught herein, particularly in a sample from the subject, (ii) optionally, where the test panel comprises a clinical parameter or parameters, means for measuring or scoring said clinical parameter or parameters (alternatively, said clinical parameter(s) may be measured or scored independently using methods and/or instruments external to the kit; in such case, the kit may contain an instruction to measure or score said clinical parameter(s)), particularly in the subject, and (iii) optionally and preferably a reference value for the test panel or means for establishing said reference value, wherein said reference value represents a known diagnosis, prediction and/or prognosis of the respective diseases or conditions.

The means for measuring the quantity of the biomarker(s) in such kits may comprise, respectively, one or more binding agents capable of specifically binding to said biomarker(s). Binding agent may be inter alia an antibody, aptamer, photoaptamer, protein, peptide, peptidomimetic or a small molecule. Preferably, the present kits comprise (i) one or more binding agents capable of specifically binding to said one or more markers as taught herein, such as one or more aptamers, antibodies, photoaptamers, proteins, peptides, peptidomimetics or small molecules, preferably one or more aptamers or antibodies, more preferably one or more aptamers capable of specifically binding to said one or more markers as taught herein. A binding agent may be advantageously immobilised on a solid phase or support. The present kits may employ an immunoassay technology or mass spectrometry analysis technology or chromatography technology, or a combination of said technologies, preferably the present kits employ an immunoassay technology, in preferred but non-limiting examples, enzyme-linked immunosorbent assay (ELISA), radioimmunoassay (RIA), or ELISPOT technologies, preferably using ELISA. Hence, the means for measuring the quantity of marker(s) may be an immunoassay, e.g., an immunoassay employing antibody(ies) and/or aptamers, e.g., ELISA, RIA, or ELISPOT assay.

Disclosed is thus also a kit, particularly a kit for the diagnosis, prediction, prognosis and/or monitoring the diseases or conditions as taught herein in a subject, the kit comprising: (i) one or more binding agents capable of specifically binding to the biomarker or biomarkers comprised in a test panel as taught herein, particularly in a sample from the subject, (ii) preferably, a known quantity or concentration of said biomarker or biomarkers (e.g., for use as controls, standards and/or calibrators), (iii) optionally, where the test panel comprises a clinical parameter or parameters, means for measuring or scoring said clinical parameter or parameters, particularly in the subject (alternatively, said clinical parameter(s) may be measured or scored independently using methods and/or instruments external to the kit; in such case, the kit may contain an instruction to measure or score said clinical parameter(s)), (iv) optionally and preferably a reference value for the test panel or means for establishing said reference value, wherein said reference value represents a known diagnosis, prediction and/or prognosis of the respective diseases or conditions. Said components under (i) and/or (ii) may be suitably labelled as taught elsewhere in this specification.

Preferred but non-limiting embodiments of the kits disclosed herein, particularly a kit for the diagnosis, prediction, prognosis and/or monitoring of a systemic inflammatory disease in a subject, may comprise: means for measuring the quantity of PCT or a fragment thereof; and means for measuring the quantity of one or more (such as, e.g., one, two, three, four or five; such as, preferably, one, two, three or four; such as, more preferably, one, two or three; such as, even more preferably, one or two) markers selected from the group consisting of PRTN3, GSHB, PTX3, VCAM1, PSA3, NID1, GOLM1, ATF6A, ICAM1, PIGR, CALU, EXT2, PHLD, FGL1, ILIR2, CATG, and ELNE, or a fragment thereof, or selected from the group consisting of PRTN3, GSHB, PTX3, VCAM1, PSA3, NID1, GOLM1, ATF6A, ICAM1, PIGR, CALU, EXT2, PHLD, FGL1, and IL1R2, or a fragment thereof.

For example, the kit may comprise: means for measuring the quantity of PCT or a fragment thereof; and means for measuring the quantity of at least two, such as exactly two, markers selected from the group consisting of PRTN3, GSHB, PTX3, VCAM1, PSA3, NID1, GOLM1, ATF6A, ICAM1, PIGR, CALU, EXT2, PHLD, FGL1, ILIR2, CATG, and ELNE, or a fragment thereof, or selected from the group consisting of PRTN3, GSHB, PTX3, VCAM1, PSA3, NID1, GOLM1, ATF6A, ICAM1, PIGR, CALU, EXT2, PHLD, FGL1, and IL1R2, or a fragment thereof.

In certain embodiments of the kit, means for measuring the quantity of PTX3 or a fragment thereof may be included in the kit instead of or in addition to, preferably instead of, means for measuring the quantity of PCT or a fragment thereof. This may particularly apply to kits which comprise at least one or preferably both of means for measuring the quantity of PRTN3 or a fragment thereof and means for measuring the quantity of GSHB or a fragment thereof, e.g., an exemplary but non-limiting kit comprising or consisting of means for measuring the quantity of PTX3, PRTN3 and GSHB.

In certain embodiments, any one of the present kits, and particularly preferably a kit comprising means for measuring the quantity of PCT or a fragment thereof and means for measuring the quantity of GSHB or a fragment thereof, may further comprise means for measuring or scoring white blood cell (WBC) count. Alternatively, said means for measuring or scoring WBC count may be measured or scored independently using methods and/or instruments external to the kit; in such case, the kit may contain an instruction to measure or score WBC.

In a further preferred example, the kit may comprise: means for measuring the quantity of PCT or PTX3, or a fragment thereof, and at least one and preferably both of means for measuring the quantity of PRTN3 or a fragment thereof and means for measuring the quantity of GSHB or a fragment thereof. In certain embodiments, the means for measuring the quantity of PRTN3 or a fragment thereof may be replaced or complemented by one or both of means for measuring the quantity of CATG or a fragment thereof and means for measuring the quantity of ELNE or a fragment thereof.

In further preferred examples, the kit may comprise: means for measuring the quantity of PCT or a fragment thereof and means for measuring the quantity of PRTN3 or a fragment thereof; or means for measuring the quantity of PCT or a fragment thereof and means for measuring the quantity of GSHB or a fragment thereof; or means for measuring the quantity of PCT or a fragment thereof, means for measuring the quantity of PRTN3 or a fragment thereof, and means for measuring the quantity of GSHB or a fragment thereof; or means for measuring the quantity of PTX3 or a fragment thereof, means for measuring the quantity of PRTN3 or a fragment thereof, and means for measuring the quantity of GSHB or a fragment thereof. In certain embodiments, the means for measuring the quantity of PRTN3 or a fragment thereof may be replaced or complemented by one or both of means for measuring the quantity of CATG or a fragment thereof and means for measuring the quantity of ELNE or a fragment thereof.

In further preferred examples, the kit may comprise: means for measuring the quantity of PCT or a fragment thereof, means for measuring the quantity of PRTN3 or a fragment thereof, and means for measuring the quantity of VCAM1 or a fragment thereof; or means for measuring the quantity of PCT or a fragment thereof, means for measuring the quantity of PRTN3 or a fragment thereof, and means for measuring the quantity of PSA3 or a fragment thereof; or means for measuring the quantity of PCT or a fragment thereof, means for measuring the quantity of PRTN3 or a fragment thereof, and means for measuring the quantity of NID1 or a fragment thereof; or means for measuring the quantity of PCT or a fragment thereof, means for measuring the quantity of PRTN3 or a fragment thereof, and means for measuring the quantity of GOLM1 or a fragment thereof; or means for measuring the quantity of PCT or a fragment thereof, means for measuring the quantity of PRTN3 or a fragment thereof, and means for measuring the quantity of PTX3 or a fragment thereof; or means for measuring the quantity of PCT or a fragment thereof, means for measuring the quantity of GSHB or a fragment thereof, and means for measuring the quantity of ATF6A or a fragment thereof; or means for measuring the quantity of PCT or a fragment thereof, means for measuring the quantity of GSHB or a fragment thereof, and means for measuring the quantity of ICAM1 or a fragment thereof; or means for measuring the quantity of PCT or a fragment thereof, means for measuring the quantity of GSHB or a fragment thereof, and means for measuring or scoring WBC or an instruction to measure or score WBC; or means for measuring the quantity of PCT or a fragment thereof, means for measuring the quantity of GSHB or a fragment thereof, and means for measuring the quantity of PIGR or a fragment thereof; or means for measuring the quantity of PCT or a fragment thereof, means for measuring the quantity of GSHB or a fragment thereof, and means for measuring the quantity of PTX3 or a fragment thereof; or means for measuring the quantity of PCT or a fragment thereof, means for measuring the quantity of GSHB or a fragment thereof, and means for measuring the quantity of CALU or a fragment thereof; or means for measuring the quantity of PCT or a fragment thereof, means for measuring the quantity of VCAM1 or a fragment thereof, and means for measuring the quantity of IL6 or a fragment thereof; or means for measuring the quantity of PCT or a fragment thereof, means for measuring the quantity of VCAM1 or a fragment thereof, and means for measuring the quantity of EXT2 or a fragment thereof; or means for measuring the quantity of PCT or a fragment thereof, means for measuring the quantity of PHLD or a fragment thereof, and means for measuring the quantity of EXT2 or a fragment thereof; or means for measuring the quantity of PCT or a fragment thereof, means for measuring the quantity of FGL1 or a fragment thereof, and means for measuring the quantity of GOLM1 or a fragment thereof; or means for measuring the quantity of PCT or a fragment thereof, means for measuring the quantity of FGL1 or a fragment thereof, and means for measuring the quantity of NID1 or a fragment thereof. In certain embodiments, the means for measuring the quantity of PRTN3 or a fragment thereof may be replaced or complemented by one or both of means for measuring the quantity of CATG or a fragment thereof and means for measuring the quantity of ELNE or a fragment thereof.

Any one kit as described herein may be suitably used for the diagnosis, prediction, prognosis and/or monitoring a systemic inflammatory condition in a subject. In some preferred embodiments, said systemic inflammatory condition may be sepsis. In other embodiments, said systemic inflammatory condition may be SIRS.

Also disclosed are reagents and tools useful for measuring any one or more markers selected from the group consisting of PRTN3, MRC1, EXT2, ILIR2, PTX3, MA1A1, ACBP, LMAN2, ACHA7, ATF6A, B4GT1, CAMP, GOLM1, NID1, MMP3, LBP, FBLN1, PIGR, TIMP1, PHLD, ANGT, CBPN, CH3L1, CSF1, DAG1, FBN1, FGL1, GSHB, ICAM1, LUM, S10A9, SAA, SRGN, VCAM1, CALU, EMAL3, GDIR2, GUC2B, HSP7C, I13R1, MOES, PDIA6, PSA3, PTPRG, S10A8, CATG, and ELNE, or a fragment thereof, or selected from the group consisting of PRTN3, MRC1, EXT2, ILIR2, PTX3, MA1A1, ACBP, LMAN2, ACHA7, ATF6A, B4GT1, CAMP, GOLM1, NID1, MMP3, LBP, FBLN1, PIGR, TIMP1, PHLD, ANGT, CBPN, CH3L1, CSF1, DAG1, FBN1, FGL1, GSHB, ICAM1, LUM, S10A9, SAA, SRGN, VCAM1, CALU, EMAL3, GDIR2, GUC2B, HSP7C, I13R1, MOES, PDIA6, PSA3, PTPRG, and S10A8, or a fragment thereof, and optionally the one or more other biomarkers concerned herein.

Also disclosed are reagents and tools useful for measuring biomarker(s) comprised in test panels as taught herein.

Hence, disclosed is a protein, polypeptide or peptide array or microarray comprising (a) any one or more markers selected from the group consisting of PRTN3, MRC1, EXT2, IL1R2, PTX3, MA1A1, ACBP, LMAN2, ACHA7, ATF6A, B4GT1, CAMP, GOLM1, NID1, MMP3, LBP, FBLN1, PIGR, TIMP1, PHLD, ANGT, CBPN, CH3L1, CSF1, DAG1, FBN1, FGL1, GSHB, ICAM1, LUM, S10A9, SAA, SRGN, VCAM1, CALU, EMAL3, GDIR2, GUC2B, HSP7C, I13R1, MOES, PDIA6, PSA3, PTPRG, S10A8, CATG, and ELNE, or a fragment thereof, or selected from the group consisting of PRTN3, MRC1, EXT2, ILIR2, PTX3, MA1A1, ACBP, LMAN2, ACHA7, ATF6A, B4GT1, CAMP, GOLM1, NID1, MMP3, LBP, FBLN1, PIGR, TIMP1, PHLD, ANGT, CBPN, CH3L1, CSF1, DAG1, FBN1, FGL1, GSHB, ICAM1, LUM, S10A9, SAA, SRGN, VCAM1, CALU, EMAL3, GDIR2, GUC2B, HSP7C, I13R1, MOES, PDIA6, PSA3, PTPRG, and S10A8, or a fragment thereof, preferably a known quantity or concentration of said one or more markers or a fragment thereof; and (b) optionally and preferably, one or more other biomarkers, preferably a known quantity or concentration of said one or more other biomarkers useful for the diagnosis, prediction and/or prognosis of the diseases or conditions as taught herein in a subject.

Further provided is the use of any one protein, polypeptide or peptide array or microarray as described herein, for the diagnosis, prediction, prognosis and/or monitoring of the respective diseases or conditions as taught herein in a subject.

Further disclosed is a protein, polypeptide or peptide array or microarray, in particular for performing the methods as taught herein, comprising the biomarkers comprised in any test panel as taught herein, preferably a known quantity or concentration of the biomarkers.

For example, certain embodiments disclose a protein, polypeptide or peptide array or microarray, in particular for performing the methods as taught herein, the protein, polypeptide or peptide array or microarray comprising (a) PCT or a fragment thereof, preferably a known quantity or concentration of PCT or a fragment thereof; (b) one or more (such as, e.g., one, two, three, four or five; such as, preferably, one, two, three or four; such as, more preferably, one, two or three; such as, even more preferably, one or two) markers selected from the group consisting of PRTN3, GSHB, PTX3, VCAM1, PSA3, NID1, GOLM1, ATF6A, ICAM1, PIGR, CALU, EXT2, PHLD, FGL1, IL1R2, CATG, and ELNE, or a fragment thereof, or selected from the group consisting of PRTN3, GSHB, PTX3, VCAM1, PSA3, NID1, GOLM1, ATF6A, ICAM1, PIGR, CALU, EXT2, PHLD, FGL1, and IL1R2, or a fragment thereof, preferably a known quantity or concentration of said one or more markers or a fragment thereof; and (c) optionally and preferably, one or more other biomarkers, preferably a known quantity or concentration of said one or more other biomarkers useful for the diagnosis, prediction and/or prognosis of the diseases or conditions as taught herein in a subject.

For example, the protein, polypeptide or peptide array or microarray may comprise: PCT or a fragment thereof; and at least two, such as exactly two, markers selected from the group consisting of PRTN3, GSHB, PTX3, VCAM1, PSA3, NID1, GOLM1, ATF6A, ICAM1, PIGR, CALU, EXT2, PHLD, FGL1, IL1R2, CATG, and ELNE, or a fragment thereof, or selected from the group consisting of PRTN3, GSHB, PTX3, VCAM1, PSA3, NID1, GOLM1, ATF6A, ICAM1, PIGR, CALU, EXT2, PHLD, FGL1, and IL1R2, or a fragment thereof.

In certain embodiments of the protein, polypeptide or peptide array or microarray, PTX3 or a fragment thereof may be included in the array or microarray instead of or in addition to, preferably instead of, PCT or a fragment thereof. This may particularly apply to arrays or microarrays which comprise at least one or preferably both of PRTN3 or a fragment thereof and GSHB or a fragment thereof, e.g., an exemplary but non-limiting array or microarray comprising or consisting of PTX3, PRTN3 and GSHB.

In a further preferred example, the array or microarray may comprise: PCT or PTX3, or a fragment thereof, and at least one and preferably both of PRTN3 or a fragment thereof and GSHB or a fragment thereof. In certain embodiments, PRTN3 or a fragment thereof may be replaced or complemented by one or both of CATG or a fragment thereof and ELNE or a fragment thereof.

In further preferred examples, the array or microarray may comprise: PCT or a fragment thereof and PRTN3 or a fragment thereof; or PCT or a fragment thereof and GSHB or a fragment thereof; or PCT or a fragment thereof, PRTN3 or a fragment thereof, and GSHB or a fragment thereof; or PTX3 or a fragment thereof, PRTN3 or a fragment thereof, and GSHB or a fragment thereof. In certain embodiments, PRTN3 or a fragment thereof may be replaced or complemented by one or both of CATG or a fragment thereof and ELNE or a fragment thereof.

In further preferred examples, the array or microarray may comprise: PCT or a fragment thereof, PRTN3 or a fragment thereof, and VCAM1 or a fragment thereof; or PCT or a fragment thereof, PRTN3 or a fragment thereof, and PSA3 or a fragment thereof; or PCT or a fragment thereof, PRTN3 or a fragment thereof, and NID1 or a fragment thereof; or PCT or a fragment thereof, PRTN3 or a fragment thereof, and GOLM1 or a fragment thereof; or PCT or a fragment thereof, PRTN3 or a fragment thereof, and PTX3 or a fragment thereof; or PCT or a fragment thereof, GSHB or a fragment thereof, and ATF6A or a fragment thereof; or PCT or a fragment thereof, GSHB or a fragment thereof, and ICAM1 or a fragment thereof; or PCT or a fragment thereof, and GSHB or a fragment thereof; or PCT or a fragment thereof, GSHB or a fragment thereof, and PIGR or a fragment thereof; or PCT or a fragment thereof, GSHB or a fragment thereof, and PTX3 or a fragment thereof; or PCT or a fragment thereof, GSHB or a fragment thereof, and CALU or a fragment thereof; or PCT or a fragment thereof, VCAM1 or a fragment thereof, and IL6 or a fragment thereof; or PCT or a fragment thereof, VCAM1 or a fragment thereof, and EXT2 or a fragment thereof; or PCT or a fragment thereof, PHLD or a fragment thereof, and EXT2 or a fragment thereof; or PCT or a fragment thereof, FGL1 or a fragment thereof, and GOLM1 or a fragment thereof; or PCT or a fragment thereof, FGL1 or a fragment thereof, and NID1 or a fragment thereof. In certain embodiments, PRTN3 or a fragment thereof may be replaced or complemented by one or both of CATG or a fragment thereof and ELNE or a fragment thereof.

Further disclosed is the use of any one protein, polypeptide or peptide array or microarray as described herein for the diagnosis, prediction, prognosis, and/or monitoring a systemic inflammatory condition in a subject. In some preferred embodiments, said systemic inflammatory condition may be sepsis. In other embodiments, said systemic inflammatory condition may be SIRS.

Also disclosed is a binding agent array or microarray comprising: (a) one or more binding agents capable of specifically binding to any one or more markers selected from the group consisting of PRTN3, MRC1, EXT2, IL1R2, PTX3, MA1A1, ACBP, LMAN2, ACHA7, ATF6A, B4GT1, CAMP, GOLM1, NID1, MMP3, LBP, FBLN1, PIGR, TIMP1, PHLD, ANGT, CBPN, CH3L1, CSF1, DAG1, FBN1, FGL1, GSHB, ICAM1, LUM, S10A9, SAA, SRGN, VCAM1, CALU, EMAL3, GDIR2, GUC2B, HSP7C, I13R1, MOES, PDIA6, PSA3, PTPRG, S10A8, CATG, and ELNE, or a fragment thereof, or selected from the group consisting of PRTN3, MRC1, EXT2, ILIR2, PTX3, MA1A1, ACBP, LMAN2, ACHA7, ATF6A, B4GT1, CAMP, GOLM1, NID1, MMP3, LBP, FBLN1, PIGR, TIMP1, PHLD, ANGT, CBPN, CH3L1, CSF1, DAG1, FBN1, FGL1, GSHB, ICAM1, LUM, S10A9, SAA, SRGN, VCAM1, CALU, EMAL3, GDIR2, GUC2B, HSP7C, I13R1, MOES, PDIA6, PSA3, PTPRG, and S10A8, or a fragment thereof, preferably a known quantity or concentration of said binding agents; and (b) optionally and preferably, one or more binding agents useful for the diagnosis, prediction and/or prognosis of the diseases or conditions as taught herein in a subject, preferably a known quantity or concentration of said binding agents. Such binding agents may be as detailed elsewhere in this specification.

Further provided is the use of any one binding agent array or microarray as described herein, for the diagnosis, prediction, prognosis and/or monitoring of the respective diseases or conditions as taught herein in a subject. In particular, disclosed is the use of any one binding agent array or microarray as described herein comprising one or more binding agents capable of specifically binding to any one or more markers selected from the group consisting of PRTN3, MRC1, EXT2, ILIR2, PTX3, MA1A1, ACBP, LMAN2, ACHA7, ATF6A, B4GT1, CAMP, GOLM1, NID1, MMP3, LBP, FBLN1, PIGR, TIMP1, PHLD, ANGT, CBPN, CH3L1, CSF1, DAG1, FBN1, FGL1, GSHB, ICAM1, LUM, S10A9, SAA, SRGN, VCAM1, CALU, EMAL3, GDIR2, GUC2B, HSP7C, I13R1, MOES, PDIA6, PSA3, PTPRG, S10A8, CATG, and ELNE, or a fragment thereof, or selected from the group consisting of PRTN3, MRC1, EXT2, ILIR2, PTX3, MA1A1, ACBP, LMAN2, ACHA7, ATF6A, B4GT1, CAMP, GOLM1, NID1, MMP3, LBP, FBLN1, PIGR, TIMP1, PHLD, ANGT, CBPN, CH3L1, CSF1, DAG1, FBN1, FGL1, GSHB, ICAM1, LUM, S10A9, SAA, SRGN, VCAM1, CALU, EMAL3, GDIR2, GUC2B, HSP7C, I13R1, MOES, PDIA6, PSA3, PTPRG, and S10A8, or a fragment thereof, in a sample from a subject, for performing any one of the methods as taught herein. Also intended herein is the use of any one binding agent array or microarray as described herein, wherein the binding agent array or microarray further comprises one or more binding agents useful for the diagnosis, prediction and/or prognosis of the diseases or conditions as taught herein in a subject, preferably a known quantity or concentration of said binding agents.

Also disclosed is a binding agent array or microarray, in particular for performing the methods as taught herein, comprising one or more binding agents capable of specifically binding to the biomarkers comprised in any test panel as taught herein, preferably a known quantity of or concentration of said binding agents. Such binding agents may be as detailed elsewhere in this specification.

For example, certain embodiments disclose a binding agent array or microarray, in particular for performing the methods as taught herein, the binding agent array comprising (a) one or more binding agents capable of specifically binding to PCT or a fragment thereof, preferably a known quantity or concentration of said binding agents; (b) one or more binding agents capable of specifically binding to one or more (such as, e.g., one, two, three, four or five; such as, preferably, one, two, three or four; such as, more preferably, one, two or three; such as, even more preferably, one or two) markers selected from the group consisting of PRTN3, GSHB, PTX3, VCAM1, PSA3, NID1, GOLM1, ATF6A, ICAM1, PIGR, CALU, EXT2, PHLD, FGL1, ILIR2, CATG, and ELNE, or a fragment thereof, or selected from the group consisting of PRTN3, GSHB, PTX3, VCAM1, PSA3, NID1, GOLM1, ATF6A, ICAM1, PIGR, CALU, EXT2, PHLD, FGL1, and IL1R2, or a fragment thereof, preferably a known quantity or concentration of said binding agents; and (c) optionally and preferably, one or more binding agents useful for the diagnosis, prediction and/or prognosis of the diseases or conditions as taught herein in a subject, preferably a known quantity or concentration of said binding agents.

For example, the binding agent array or microarray may comprise: one or more binding agents capable of specifically binding to PCT or a fragment thereof; and one or more binding agents capable of specifically binding to at least two, such as exactly two, markers selected from the group consisting of PRTN3, GSHB, PTX3, VCAM1, PSA3, NID1, GOLM1, ATF6A, ICAM1, PIGR, CALU, EXT2, PHLD, FGL1, ILIR2, CATG, and ELNE, or a fragment thereof, or selected from the group consisting of PRTN3, GSHB, PTX3, VCAM1, PSA3, NID1, GOLM1, ATF6A, ICAM1, PIGR, CALU, EXT2, PHLD, FGL1, and IL1R2, or a fragment thereof.

In certain embodiments of the binding agent array or microarray, one or more binding agents capable of specifically binding to PTX3 or a fragment thereof may be included in the array or microarray instead of or in addition to, preferably instead of, one or more binding agents capable of specifically binding to PCT or a fragment thereof. This may particularly apply to arrays or microarrays which comprise at least one or preferably both of one or more binding agents capable of specifically binding to PRTN3 or a fragment thereof and one or more binding agents capable of specifically binding to GSHB or a fragment thereof, e.g., an exemplary but non-limiting array or microarray comprising or consisting of one or more binding agents capable of specifically binding to PTX3, one or more binding agents capable of specifically binding to PRTN3 and one or more binding agents capable of specifically binding to GSHB.

In a further preferred example, the binding agent array or microarray may comprise: one or more binding agents capable of specifically binding to PCT or one or more binding agents capable of specifically binding to PTX3, or a fragment thereof, and at least one and preferably both of one or more binding agents capable of specifically binding to PRTN3 or a fragment thereof and one or more binding agents capable of specifically binding to GSHB or a fragment thereof. In certain embodiments, one or more binding agents capable of specifically binding to PRTN3 or a fragment thereof may be replaced or complemented by one or both of one or more binding agents capable of specifically binding to CATG or a fragment thereof and one or more binding agents capable of specifically binding to ELNE or a fragment thereof.

In further preferred examples, the binding agent array or microarray may comprise: one or more binding agents capable of specifically binding to PCT or a fragment thereof and one or more binding agents capable of specifically binding to PRTN3 or a fragment thereof; or one or more binding agents capable of specifically binding to PCT or a fragment thereof and one or more binding agents capable of specifically binding to GSHB or a fragment thereof; or one or more binding agents capable of specifically binding to PCT or a fragment thereof, one or more binding agents capable of specifically binding to PRTN3 or a fragment thereof, and one or more binding agents capable of specifically binding to GSHB or a fragment thereof; or one or more binding agents capable of specifically binding to PTX3 or a fragment thereof, one or more binding agents capable of specifically binding to PRTN3 or a fragment thereof, and one or more binding agents capable of specifically binding to GSHB or a fragment thereof. In certain embodiments, one or more binding agents capable of specifically binding to PRTN3 or a fragment thereof may be replaced or complemented by one or both of one or more binding agents capable of specifically binding to CATG or a fragment thereof and one or more binding agents capable of specifically binding to ELNE or a fragment thereof.

In further preferred examples, the binding agent array or microarray may comprise: one or more binding agents capable of specifically binding to one or more binding agents capable of specifically binding to PCT or a fragment thereof, one or more binding agents capable of specifically binding to one or more binding agents capable of specifically binding to PRTN3 or a fragment thereof, and one or more binding agents capable of specifically binding to one or more binding agents capable of specifically binding to VCAM1 or a fragment thereof; or one or more binding agents capable of specifically binding to PCT or a fragment thereof, one or more binding agents capable of specifically binding to PRTN3 or a fragment thereof, and one or more binding agents capable of specifically binding to PSA3 or a fragment thereof; or one or more binding agents capable of specifically binding to PCT or a fragment thereof, one or more binding agents capable of specifically binding to PRTN3 or a fragment thereof, and one or more binding agents capable of specifically binding to NID1 or a fragment thereof; or one or more binding agents capable of specifically binding to PCT or a fragment thereof, one or more binding agents capable of specifically binding to PRTN3 or a fragment thereof, and one or more binding agents capable of specifically binding to GOLM1 or a fragment thereof; or one or more binding agents capable of specifically binding to PCT or a fragment thereof, one or more binding agents capable of specifically binding to PRTN3 or a fragment thereof, and one or more binding agents capable of specifically binding to PTX3 or a fragment thereof; or one or more binding agents capable of specifically binding to PCT or a fragment thereof, one or more binding agents capable of specifically binding to GSHB or a fragment thereof, and one or more binding agents capable of specifically binding to ATF6A or a fragment thereof; or one or more binding agents capable of specifically binding to PCT or a fragment thereof, one or more binding agents capable of specifically binding to GSHB or a fragment thereof, and one or more binding agents capable of specifically binding to ICAM1 or a fragment thereof; or one or more binding agents capable of specifically binding to PCT or a fragment thereof, one or more binding agents capable of specifically binding to GSHB or a fragment thereof; or one or more binding agents capable of specifically binding to PCT or a fragment thereof, one or more binding agents capable of specifically binding to GSHB or a fragment thereof, and one or more binding agents capable of specifically binding to PIGR or a fragment thereof; or one or more binding agents capable of specifically binding to PCT or a fragment thereof, one or more binding agents capable of specifically binding to GSHB or a fragment thereof, and one or more binding agents capable of specifically binding to PTX3 or a fragment thereof; or one or more binding agents capable of specifically binding to PCT or a fragment thereof, one or more binding agents capable of specifically binding to GSHB or a fragment thereof, and one or more binding agents capable of specifically binding to CALU or a fragment thereof; or one or more binding agents capable of specifically binding to PCT or a fragment thereof, one or more binding agents capable of specifically binding to VCAM1 or a fragment thereof, and one or more binding agents capable of specifically binding to IL6 or a fragment thereof; or one or more binding agents capable of specifically binding to PCT or a fragment thereof, one or more binding agents capable of specifically binding to VCAM1 or a fragment thereof, and one or more binding agents capable of specifically binding to EXT2 or a fragment thereof; or one or more binding agents capable of specifically binding to PCT or a fragment thereof, one or more binding agents capable of specifically binding to PHLD or a fragment thereof, and one or more binding agents capable of specifically binding to EXT2 or a fragment thereof; or one or more binding agents capable of specifically binding to PCT or a fragment thereof, one or more binding agents capable of specifically binding to FGL1 or a fragment thereof, and one or more binding agents capable of specifically binding to GOLM1 or a fragment thereof; or one or more binding agents capable of specifically binding to PCT or a fragment thereof, one or more binding agents capable of specifically binding to FGL1 or a fragment thereof, and one or more binding agents capable of specifically binding to NID1 or a fragment thereof. In certain embodiments, one or more binding agents capable of specifically binding to PRTN3 or a fragment thereof may be replaced or complemented by one or both of one or more binding agents capable of specifically binding to CATG or a fragment thereof and one or more binding agents capable of specifically binding to ELNE or a fragment thereof.

Further disclosed is the use of any one binding agent array or microarray as described herein for the diagnosis, prediction, prognosis, and/or monitoring a systemic inflammatory condition in a subject. In some preferred embodiments, said systemic inflammatory condition may be sepsis. In other embodiments, said systemic inflammatory condition may be SIRS.

Also disclosed are kits as taught here above configured as portable devices, such as, for example, bed-side devices.

A related aspect thus provides a portable testing device capable of measuring the quantity of any one or more markers selected from the group consisting of PRTN3, MRC1, EXT2, IL1R2, PTX3, MA1A1, ACBP, LMAN2, ACHA7, ATF6A, B4GT1, CAMP, GOLM1, NID1, MMP3, LBP, FBLN1, PIGR, TIMP1, PHLD, ANGT, CBPN, CH3L1, CSF1, DAG1, FBN1, FGL1, GSHB, ICAM1, LUM, S10A9, SAA, SRGN, VCAM1, CALU, EMAL3, GDIR2, GUC2B, HSP7C, I13R1, MOES, PDIA6, PSA3, PTPRG, S10A8, CATG, and ELNE, or a fragment thereof, or selected from the group consisting of PRTN3, MRC1, EXT2, ILIR2, PTX3, MA1A1, ACBP, LMAN2, ACHA7, ATF6A, B4GT1, CAMP, GOLM1, NID1, MMP3, LBP, FBLN1, PIGR, TIMP1, PHLD, ANGT, CBPN, CH3L1, CSF1, DAG1, FBN1, FGL1, GSHB, ICAM1, LUM, S10A9, SAA, SRGN, VCAM1, CALU, EMAL3, GDIR2, GUC2B, HSP7C, I13R1, MOES, PDIA6, PSA3, PTPRG, and S10A8, or a fragment thereof, in a sample from a subject comprising: (i) means for obtaining a sample from the subject, (ii) means for measuring the quantity of said one or more markers or a fragment thereof in said sample, and (iii) means for visualising the quantity of said one or more markers or a fragment thereof measured in the sample.

In an embodiment, the means of parts (ii) and (iii) may be the same, thus providing a portable testing device capable of measuring the quantity of said one or more markers or a fragment thereof in a sample from a subject comprising (i) means for obtaining a sample from the subject; and (ii) means for measuring the quantity of said one or more markers or a fragment thereof in said sample and visualising the quantity of said one or more markers or a fragment thereof measured in the sample.

In an embodiment, said visualising means is capable of indicating whether the quantity of said one or more markers or a fragment thereof in the sample is above or below a certain threshold level and/or whether the quantity of said one or more markers or a fragment thereof in the sample deviates or not from a reference value of the quantity of said one or more markers or a fragment thereof, said reference value representing a known diagnosis, prediction and/or prognosis of the diseases or conditions as taught herein. Hence, the portable testing device may suitably also comprise said reference value or means for establishing the reference value.

A further related aspect thus provides a portable testing device capable of measuring the quantity of the biomarker or biomarkers comprised in any test panel as taught herein in a sample from a subject comprising: (i) means for obtaining a sample from the subject, (ii) means for measuring the quantity of the biomarker or biomarkers comprised in the test panel in said sample, and (iii) means for visualising the quantity of said biomarker or biomarkers in the sample. Where the test panel comprises a clinical parameter or parameters, the testing device may optionally further comprise (iv) means for measuring or scoring the parameter or parameters comprised in the test panel in the subject and (v) means for visualising the measurement or score of said parameter or parameters in the subject (alternatively, said clinical parameter(s) may be measured or scored independently using methods and/or instruments external to the portable testing device; in such case, the portable testing device package may contain an instruction to measure or score said clinical parameter(s)). In an embodiment, the means of parts (ii) and (iii) may be the same. In an embodiment, the means of parts (iii) and (v) may be the same.

In an embodiment, said visualising means is capable of indicating whether the quantity of the biomarker or biomarkers and the measurement or score of the parameter or parameters in the subject deviates from (e.g., is below or above) a certain reference or base-line value as taught herein. Hence, the portable testing device may suitably also comprise said reference or base-line value or means for establishing the same.

Other aspects of the present invention relate to the realisation that markers disclosed herein may be valuable targets for therapeutic and/or prophylactic interventions in diseases and conditions as taught herein, in particular systemic inflammatory conditions, including SIRS and sepsis.

Hence, also disclosed herein are any one and all of the following:

(1) an agent that is able to modulate the level and/or the activity of any one or more nucleic acids or proteins selected from the group consisting of PRTN3, MRC1, EXT2, ILIR2, PTX3, MA1A1, ACBP, LMAN2, ACHA7, ATF6A, B4GT1, CAMP, GOLM1, NID1, MMP3, LBP, FBLN1, PIGR, TIMP1, PHLD, ANGT, CBPN, CH3L1, CSF1, DAG1, FBN1, FGL1, GSHB, ICAM1, LUM, S10A9, SAA, SRGN, VCAM1, CALU, EMAL3, GDIR2, GUC2B, HSP7C, I13R1, MOES, PDIA6, PSA3, PTPRG, S10A8, CATG, and ELNE, or a fragment thereof, or selected from the group consisting of PRTN3, MRC1, EXT2, ILIR2, PTX3, MA1A1, ACBP, LMAN2, ACHA7, ATF6A, B4GT1, CAMP, GOLM1, NID1, MMP3, LBP, FBLN1, PIGR, TIMP1, PHLD, ANGT, CBPN, CH3L1, CSF1, DAG1, FBN1, FGL1, GSHB, ICAM1, LUM, S10A9, SAA, SRGN, VCAM1, CALU, EMAL3, GDIR2, GUC2B, HSP7C, I13R1, MOES, PDIA6, PSA3, PTPRG, and S10A8, or a fragment thereof, for use as a medicament, preferably for use in the treatment of any one disease or condition as taught herein;
(2) use of an agent that is able to modulate the level and/or the activity of said one or more nucleic acids or proteins as defined in (1) above for the manufacture of a medicament for the treatment of any one disease or condition as taught herein; or use of an agent that is able to modulate the level and/or the activity of said one or more nucleic acids or proteins as defined in (1) above for the treatment of any one disease or condition as taught herein;
(3) a method for treating any one disease or condition as taught herein in a subject in need of such treatment, comprising administering to said subject a therapeutically or prophylactically effective amount of an agent that is able to modulate the level and/or the activity of said one or more nucleic acids or proteins as defined in (1) above;
(4) The subject matter as set forth in any one of (1) to (3) above, wherein the agent is able to reduce or increase the level and/or the activity of said one or more nucleic acids or proteins as defined in (1) above.
(5) The subject matter as set forth in any one of (1) to (4) above, wherein said agent is able to specifically bind to said one or more nucleic acids or proteins as defined in (1) above.
(6) The subject matter as set forth in any one of (1) to (5) above, wherein said agent is an antibody or a fragment or derivative thereof; a polypeptide; a peptide; a peptidomimetic; an aptamer; a photoaptamer; or a chemical substance, preferably an organic molecule, more preferably a small organic molecule.
(7) The subject matter as set forth in any one of (1) to (4) above, wherein the agent is able to reduce or inhibit the expression of said one or more nucleic acids or proteins as defined in (1) above, preferably wherein said agent is an antisense agent; a ribozyme; or an agent capable of causing RNA interference.
(8) The subject matter as set forth in any one of (1) to (4) above, wherein said agent is able to reduce or inhibit the level and/or activity of said one or more nucleic acids or proteins as defined in (1) above, preferably wherein said agent is a recombinant or isolated deletion construct of the said one or more proteins as defined in (1) above polypeptide having a dominant negative activity over the native one or more proteins as defined in (1) above.
(9) An assay to select, from a group of test agents, a candidate agent potentially useful in the treatment of any one disease or condition as taught herein, said assay comprising determining whether a tested agent can modulate, such as increase or reduce and preferably reduce, the level and/or activity of said one or more nucleic acids or proteins as defined in (1) above.
(10) The assay as set forth in (9) above, further comprising use of the selected candidate agent for the preparation of a composition for administration to and monitoring the prophylactic and/or therapeutic effect thereof in a non-human animal model, preferably a non-human mammal model, of any one disease or condition as taught herein.
(11) The agent isolated by the assay as set forth in (10) above.
(12) A pharmaceutical composition or formulation comprising a prophylactically and/or therapeutically effective amount of one or more agents as set forth in any one of (1) to (8) or (10) above, or a pharmaceutically acceptable N-oxide form, addition salt, prodrug or solvate thereof, and further comprising one or more of pharmaceutically acceptable carriers.
(13) A method for producing the pharmaceutical composition or formulation as set forth in (12) above, comprising admixing said one or more agents with said one or more pharmaceutically acceptable carriers.

Said condition or disease as set forth in any one of (1) to (13) above may be particularly systemic inflammatory conditions, including SIRS and sepsis.

Also contemplated is thus a method (a screening assay) for selecting an agent capable of specifically binding to any one or more nucleic acids or proteins selected from the group consisting of PRTN3, MRC1, EXT2, IL1R2, PTX3, MA1A1, ACBP, LMAN2, ACHA7, ATF6A, B4GT1, CAMP, GOLM1, NID1, MMP3, LBP, FBLN1, PIGR, TIMP1, PHLD, ANGT, CBPN, CH3L1, CSF1, DAG1, FBN1, FGL1, GSHB, ICAM1, LUM, S10A9, SAA, SRGN, VCAM1, CALU, EMAL3, GDIR2, GUC2B, HSP7C, I13R1, MOES, PDIA6, PSA3, PTPRG, S10A8, CATG, and ELNE, or a fragment thereof, or selected from the group consisting of PRTN3, MRC1, EXT2, ILIR2, PTX3, MA1A1, ACBP, LMAN2, ACHA7, ATF6A, B4GT1, CAMP, GOLM1, NID1, MMP3, LBP, FBLN1, PIGR, TIMP1, PHLD, ANGT, CBPN, CH3L1, CSF1, DAG1, FBN1, FGL1, GSHB, ICAM1, LUM, S10A9, SAA, SRGN, VCAM1, CALU, EMAL3, GDIR2, GUC2B, HSP7C, I13R1, MOES, PDIA6, PSA3, PTPRG, and S10A8, or a fragment thereof (e.g., nucleic acid such as gene, or protein) comprising: (a) providing one or more, preferably a plurality of, test binding agents; (b) selecting from the test binding agents of (a) those which bind to said one or more nucleic acids or proteins; and (c) counter-selecting (i.e., removing) from the test binding agents selected in (b) those which bind to any one or more other, unintended or undesired, targets.

Binding between test binding agents and said one or more nucleic acids or proteins may be advantageously tested by contacting (i.e., combining, exposing or incubating) said one or more nucleic acids or proteins with the test binding agents under conditions generally conducive for such binding. For example and without limitation, binding between test binding agents and said one or more nucleic acids or proteins may be suitably tested in vitro; or may be tested in host cells or host organisms comprising said one or more nucleic acids or proteins and exposed to or configured to express the test binding agents.

Without limitation, the binding or modulating agents may be capable of binding said one or more nucleic acids or proteins or modulating the activity and/or level of said one or more nucleic acids or proteins in vitro, in a cell, in an organ and/or in an organism.

In the screening assays as set forth in any one of (9) and (10) above, modulation of the activity and/or level of said one or more nucleic acids or proteins by test modulating agents may be advantageously tested by contacting (i.e., combining, exposing or incubating) said one or more nucleic acids or proteins (e.g., gene or protein) with the test modulating agents under conditions generally conducive for such modulation. By means of example and not limitation, where modulation of the activity and/or level of said one or more nucleic acids or proteins results from binding of the test modulating agents to said one or more nucleic acids or proteins, said conditions may be generally conducive for such binding. For example and without limitation, modulation of the activity and/or level of said one or more nucleic acids or proteins by test modulating agents may be suitably tested in vitro; or may be tested in host cells or host organisms comprising said one or more nucleic acids or proteins and exposed to or configured to express the test modulating agents.

Also contemplated are:
any one or more of PRTN3, MRC1, EXT2, ILIR2, PTX3, MA1A1, ACBP, LMAN2, ACHA7, ATF6A, B4GT1, CAMP, GOLM1, NID1, MMP3, LBP, FBLN1, PIGR, TIMP1, PHLD, ANGT, CBPN, CH3L1, CSF1, DAG1, FBN1, FGL1, GSHB, ICAM1, LUM, S10A9, SAA, SRGN, VCAM1, CALU, EMAL3, GDIR2, GUC2B, HSP7C, I13R1, MOES, PDIA6, PSA3, PTPRG, S10A8, CATG, and ELNE, or a fragment thereof, or selected from the group consisting of PRTN3, MRC1, EXT2, ILIR2, PTX3, MA1A1, ACBP, LMAN2, ACHA7, ATF6A, B4GT1, CAMP, GOLM1, NID1, MMP3, LBP, FBLN1, PIGR, TIMP1, PHLD, ANGT, CBPN, CH3L1, CSF1, DAG1, FBN1, FGL1, GSHB, ICAM1, LUM, S10A9, SAA, SRGN, VCAM1, CALU, EMAL3, GDIR2, GUC2B, HSP7C, I13R1, MOES, PDIA6, PSA3, PTPRG, and S10A8, or a fragment thereof (e.g., nucleic acid such as a gene, or a polypeptide or protein) for use as a medicament, preferably for use in the treatment of any one disease or condition as taught herein;
use of said one or more nucleic acids or proteins for the manufacture of a medicament for the treatment of any one disease or condition as taught herein;
use of said one or more nucleic acids or proteins for the treatment of any one disease or condition as taught herein;
a method for treating any one disease or condition as taught herein in a subject in need of such treatment, comprising administering to said subject a therapeutically or prophylactically effective amount of said one or more nucleic acids or proteins;
   particularly wherein said condition or disease may be systemic inflammatory conditions, including SIRS and sepsis.

The above and further aspects and preferred embodiments of the invention are described in the following sections and in the appended claims. The subject matter of appended claims is hereby specifically incorporated in this specification.

### BRIEF DESCRIPTION OF THE FIGURES

FIG. 1 illustrates sequences of full length PRTN3 (SEQ ID NO: 1). MRC 1 (SEQ ID NO: 3), PTX3 (SEQ ID NO: 5), IL1R2 (SEQ ID NO: 7), EXT2 (SEQ ID NO: 10), GSHB (SEQ ID NO: 13), VCAM1 (SEQ ID NO: 16), PSA3 (SEQ ID NO: 18), NID1 (SEQ ID NO: 20), GOLM1 (SEQ ID NO: 22), ATF6A (SEQ ID NO: 24), ICAM1 (SEQ ID NO: 26), PIGR (SEQ ID NO: 28), CALU (SEQ ID NO: 30), PHLD (SEQ ID NO: 32) and FGL1 (SEQ ID NO: 34). The respective peptides detected by the MASSterclass® technology are depicted as SEQ ID NO: 2, 4, 6, 8, 9, 11, 12, 14, 15, 17, 19, 21, 23, 25, 27, 29, 31, 33 and 35, respectively.

FIG. 2 left panel and right panel represent graphs illustrating box and whisker plots for PCT or PRTN3 levels, respectively, in patients of an exemplary patient cohort having SIRS, mild sepsis or severe sepsis. Mild sepsis and severe sepsis are denoted “All sepsis”.

FIG. 3 represents a graph illustrating the suitability of PRTN 3 for detecting sepsis, particularly including mild sepsis, in an exemplary patient cohort. Sensitivity and specificity of PRTN3 and PCT at maximum accuracy are shown as well as sensitivity and specificity of PCT at its clinically used cut-off of 2 ng/mL.

FIG. 4 represents a graph illustrating box and whisker plots for MRC1 levels in non-survivors with sepsis, survivors with sepsis, non-survivors with SIRS and survivors with SIRS one month after follow-up of the patients of an exemplary cohort.

FIG. 5 represents a graph illustrating box and whisker plots for EXT2 levels in patients of an exemplary patient cohort having SIRS, sepsis or severe sepsis.

### DETAILED DESCRIPTION

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The term also encompasses “consisting of” and “consisting essentially of”.

The recitation of numerical ranges by endpoints includes all numbers and fractions subsumed within the respective ranges, as well as the recited endpoints.

The term “about” as used herein when referring to a measurable value such as a parameter, an amount, a temporal duration, and the like, is meant to encompass variations of and from the specified value, in particular variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” refers is itself also specifically, and preferably, disclosed.

Whereas the term “one or more”, such as one or more members of a group of members, is clear per se, by means of further exemplification, the term encompasses inter alia a reference to any one of said members, or to any two or more of said members, such as, e.g., any ≧3, ≧4, ≧5, ≧6, or ≧7 etc. of said members, and up to all said members.

All documents cited in the present specification are hereby incorporated by reference in their entirety.

Unless otherwise specified, all terms used in disclosing the invention, including technical and scientific terms, have the meaning as commonly understood by one of ordinary skill in the art to which this invention belongs. By means of further guidance, term definitions may be included to better appreciate the teaching of the present invention.

As noted, based on extensive testing, the inventors identified any one or more of PRTN3, MRC1, EXT2, ILIR2, PTX3, MA1A1, ACBP, LMAN2, ACHA7, ATF6A, B4GT1, CAMP, GOLM1, NID1, MMP3, LBP, FBLN1, PIGR, TIMP1, PHLD, ANGT, CBPN, CH3L1, CSF1, DAG1, FBN1, FGL1, GSHB, ICAM1, LUM, S10A9, SAA, SRGN, VCAM1, CALU, EMAL3, GDIR2, GUC2B, HSP7C, I13R1, MOES, PDIA6, PSA3, PTPRG, S10A8 CATG, and ELNE, or any one or more of PRTN3, MRC1, EXT2, IL1R2, PTX3, MA1A1, ACBP, LMAN2, ACHA7, ATF6A, B4GT1, CAMP, GOLM1, NID1, MMP3, LBP, FBLN1, PIGR, TIMP1, PHLD, ANGT, CBPN, CH3L1, CSF1, DAG1, FBN1, FGL1, GSHB, ICAM1, LUM, S10A9, SAA, SRGN, VCAM1, CALU, EMAL3, GDIR2, GUC2B, HSP7C, I13R1, MOES, PDIA6, PSA3, PTPRG, and S10A8, as valuable biomarkers for evaluating various aspects of systemic inflammatory conditions such as sepsis in subjects.

The term “biomarker” is widespread in the art and may broadly denote a biological molecule and/or a detectable portion thereof whose qualitative and/or quantitative evaluation in a subject is predictive or informative (e.g., predictive, diagnostic and/or prognostic) with respect to one or more aspects of the subject's phenotype and/or genotype, such as, for example, with respect to the status of the subject as to a given disease or condition. Preferably, biomarkers as intended herein are peptide-, polypeptide- and/or protein-based. The terms “biomarker” and “marker” may be used interchangeably herein.

Furthermore, the inventors identified test panels comprising biomarker and potentially clinical parameter(s) useful in diagnosis, prognosis, prediction and/or monitoring of systemic inflammatory conditions such as sepsis in subjects.

The term “panel” or “test panel” as used herein broadly refers to combinations, sets or groupings of biomarkers and/or clinical parameters, particularly where the testing or evaluation of such panels in subjects is predictive and/or informative as regards the subject's status, disease or condition. Without limitation, a panel as intended herein may comprise or consist of between 2 and 10, preferably between 3 and 8 biomarkers and parameters, e.g., of two or three biomarkers.

The term “parameter” or “clinical parameter” is widespread in the art and may broadly denote information about a subject that is obtained in a clinical setting that may be relevant to a disease or condition of the subject. Particularly, parameters may encompass non-sample and/or non-analyte information. By means of illustration, clinical parameters common in medical practice may including inter alia basic subject characteristics such as white blood cell (WBC) count, kidney function parameters, respiratory system function, nervous system function, cardiovascular function, liver function, and coagulation function.

Sepsis may be characterized as mild sepsis, severe sepsis (sepsis with acute organ dysfunction), septic shock (sepsis with refractory arterial hypotension), organ failure, multiple organ dysfunction syndrome and death.

“Sepsis” can generally be defined as SIRS with a documented infection, such as for example a bacterial infection. Infection can be diagnosed by standard textbook criteria or, in case of uncertainty, by an infectious disease specialist. Bacteraemia is defined as sepsis where bacteria can be cultured from blood.

“SIRS” is a systemic inflammatory response syndrome with no signs of infection. It can be characterized by the presence of at least two of the four following clinical criteria: fever or hypothermia (temperature of 38.0° C. (100.4° F.) or more, or temperature of 36.0° C. (96.8° F.) or less); tachycardia (at least 90 beats per minute); tachypnea (at least 20 breaths per minute or PaCO₂ less than 4.3 kPa (32.0 mm Hg) or the need for mechanical ventilation); and an altered white blood cell (WBC) count of 12×10⁶ cells/mL or more, or an altered WBC count of 4×10⁶ cells/mL or less, or the presence of more than 10% band forms.

“Mild sepsis” can be defined as the presence of sepsis without organ dysfunction.

“Severe sepsis” can be defined as the presence of sepsis and at least one of the following manifestations of organ hypoperfusion or dysfunction: hypoxemia, metabolic acidosis, oliguria, lactic acidosis, or an acute alteration in mental status without sedation.

“Septic shock” can be defined as the presence of sepsis accompanied by a sustained decrease in systolic blood pressure (90 mm Hg or less, or a drop of at least 40 mm Hg from baseline systolic blood pressure) despite fluid resuscitation, and the need for vasoactive amines to maintain adequate blood pressure.

Common sepsis-related definitions as may also be relied on here are further detailed in Levy M M et al., Crit. Care Med., 2003, vol. 31, 1250-56, or the definitions provided by the American College of Chest Physicians and the Society of Critical Care Medicine, Crit. Care Med., 1992, vol. 20: 864-874.

As many organisms may be the cause of sepsis, diagnosis often takes time and requires testing against panels of possible agents. Sepsis may also arise in many different circumstances and therefore sepsis may be further classified for example in: incarcerated sepsis which is an infection that is latent after the primary lesion has apparently healed but may be activated by a slight trauma; catheter sepsis which is sepsis occurring as a complication of intravenous catheterization; oral sepsis which is a disease condition in the mouth or adjacent parts which may affect the general health through the dissemination of toxins; puerperal sepsis which is infection of the female genital tract following childbirth, abortion, or miscarriage; or sepsis lenta, which is a condition produced by infection with a-hemolytic streptococci, characterized by a febrile illness with endocarditis.

The term “systemic inflammatory condition” as meant herein generally encompasses diseases and conditions comprising systemic inflammatory responses. The term particularly encompasses SIRS and sepsis and may more particularly refer to SIRS and/or sepsis.

For the purposes of this invention, the reference to a disease and/or condition is meant to include all stages of the progression of the disease and/or condition.

“Organ failure” may be defined as a condition where an organ does not perform its expected function. Organ failure relates to organ dysfunction to such a degree that normal homeostasis cannot be maintained without external clinical intervention. Examples of organ failure include without limitation renal failure, (acute) liver failure, heart failure, and respiratory failure.

“Multiple organ dysfunction syndrome” (MODS), “multiple organ failure” (MOF) or “multisystem organ failure” (MSOF) may be defined as altered organ function in an acutely ill patient requiring medical intervention to achieve homeostasis. It usually involves two or more organs or organ systems.

The terms “mortality” and “death” are well known per se and herein particularly relate to outcomes indicating that a subject may (e.g., with certain likelihood) or will die (i.e., permanent termination of the biological functions that sustain a living organism), particularly that the subject may or will die as a consequence of the disease or condition and/or that he/she will die within a given time period from sampling, typically a relatively short period, such as several hours (e.g., between 1 and 24 hours or between 12 and 24 hours), several days (e.g., between 1 and 50 days or between 1 and 30 days), such as, for example within a month or within 4 weeks (28 days) from sampling.

The terms “predicting” or “prediction”, “diagnosing” or “diagnosis” and “prognosticating” or “prognosis” are commonplace and well-understood in medical and clinical practice. It shall be understood that the phrase “a method for the diagnosis, prediction and/or prognosis” a given disease or condition may also be interchanged with phrases such as “a method for diagnosing, predicting and/or prognosticating” of said disease or condition or “a method for making (or determining or establishing) the diagnosis, prediction and/or prognosis” of said disease or condition, or the like.

By means of further explanation and without limitation, “predicting” or “prediction” generally refer to an advance declaration, indication or foretelling of a disease or condition in a subject not (yet) having said disease or condition. For example, a prediction of a disease or condition in a subject may indicate a probability, chance or risk that the subject will develop said disease or condition, for example within a certain time period or by a certain age. Said probability, chance or risk may be indicated inter alia as an absolute value, range or statistics, or may be indicated relative to a suitable control subject or subject population (such as, e.g., relative to a general, normal or healthy subject or subject population). Hence, the probability, chance or risk that a subject will develop a disease or condition may be advantageously indicated as increased or decreased, or as fold-increased or fold-decreased relative to a suitable control subject or subject population. As used herein, the term “prediction” of the conditions or diseases as taught herein in a subject may also particularly mean that the subject has a ‘positive’ prediction of such, i.e., that the subject is at risk of having such (e.g., the risk is significantly increased vis-à-vis a control subject or subject population). The term “prediction of no” diseases or conditions as taught herein as described herein in a subject may particularly mean that the subject has a ‘negative’ prediction of such, i.e., that the subject's risk of having such is not significantly increased vis-à-vis a control subject or subject population.

The terms “diagnosing” or “diagnosis” generally refer to the process or act of recognising, deciding on or concluding on a disease or condition in a subject on the basis of symptoms and signs and/or from results of various diagnostic procedures (such as, for example, from knowing the presence, absence and/or quantity of one or more biomarkers characteristic of the diagnosed disease or condition). As used herein, “diagnosis of” the diseases or conditions as taught herein in a subject may particularly mean that the subject has such, hence, is diagnosed as having such. “Diagnosis of no” diseases or conditions as taught herein in a subject may particularly mean that the subject does not have such, hence, is diagnosed as not having such. A subject may be diagnosed as not having such despite displaying one or more conventional symptoms or signs reminiscent of such.

The terms “prognosticating” or “prognosis” generally refer to an anticipation on the progression of a disease or condition and the prospect (e.g., the probability, duration, and/or extent) of recovery. A good prognosis of the diseases or conditions taught herein may generally encompass anticipation of a satisfactory partial or complete recovery from the diseases or conditions, preferably within an acceptable time period. A good prognosis of such may more commonly encompass anticipation of not further worsening or aggravating of such, preferably within a given time period. A poor prognosis of the diseases or conditions as taught herein may generally encompass anticipation of a substandard recovery and/or unsatisfactorily slow recovery, or to substantially no recovery or even further worsening of such.

Hence, prediction or prognosis of a disease or condition may inter alia allow the prediction or prognosis of the occurrence of the disease or condition, or the prediction or prognosis of the progression, aggravation, alleviation or recurrence of the disease or condition or response to treatment or to other external or internal factors, situations or stressors, etc.

Further, monitoring a disease or condition may inter alia allow the prediction of the occurrence of the disease or condition, or the monitoring of the progression, aggravation, alleviation or recurrence of the disease or condition, or response to treatment or to other external or internal factors, situations or stressors, etc. Advantageously, monitoring may be applied in the course of a medical treatment of a subject, preferably medical treatment aimed at alleviating the so-monitored disease or condition. Such monitoring may be comprised, e.g., in decision making whether a patient may be discharged, needs a change in treatment or needs further hospitalisation. As intended herein, a reference to monitoring of a disease or condition also specifically includes monitoring of the probability, risk or chance of a subject to develop the disease or condition, i.e., monitoring change(s) in said probability, risk or chance over time.

The term “subject” or “patient” as used herein typically denotes humans, but may also encompass reference to non-human animals, preferably warm-blooded animals, even more preferably mammals, such as, e.g., non-human primates, rodents, canines, felines, equines, ovines, porcines, and the like. Subjects typically include both male and female genders.

In certain preferred embodiments, the present uses or methods may be particularly applied to critically ill patients. The term “critically ill subject” may be used interchangeably herein with the recitations “subject with a condition requiring critical care”, “subject with a critical illness” or “subject with a critical care condition”.

The terms “critically ill”, “critical illness”, “condition which requires critical care”, or “critical care condition” are used interchangeably herein and generally refer to a condition which is life threatening to the sufferer and may thus result in death within a relatively short period of time such as within hours or days. Such conditions require critical care (e.g. monitoring and treatment) that generally involves close, constant attention by a team of specially trained health professionals. Such care usually takes place in an intensive care unit (ICU), emergency department (ED) or trauma centre. However, care might take place in any appropriate unit which has a similar or equivalent structure and capability as an ICU, ED or trauma centre. Thus, preferred critical conditions for application of the uses or methods of the present invention are conditions requiring admittance to an ICU, ED or a setting which has a similar or equivalent structure and capability such as a trauma centre and preferred patients are ICU patients, ED patients or trauma centre patients.

Such critical care conditions include complications from surgery, life threatening accidents or other life threatening physical trauma or stress; medical shock i.e., a condition when insufficient blood flow reaches body tissues; infections e.g., bacterial, fungal or viral infections; systemic inflammatory response syndrome (SIRS); sepsis; severe sepsis i.e. sepsis with organ dysfunction; septic shock i.e., sepsis with acute circulatory failure; Acute Respiratory Distress Syndrome (ARDS) defined by pulmonary and systemic inflammation and pulmonary tissue injury (including endothelial and/or epithelial tissue) injury that result in alveolar filling and respiratory failure (Bajwa et al., Crit. Care Med., 2007, 35, 2484-2490); severe pneumonia; respiratory failure particularly acute respiratory failure; respiratory distress; severe chronic obstructive pulmonary disease (COPD); subarachnoidal hemorrhage (SAH); (severe) stroke; asphyxia; neurological conditions; organ dysfunction; single or multiple organ failure (MOF); poisoning and intoxication; severe allergic reactions and anaphylaxis; acute gastrointestinal and abdominal conditions resulting in SIRS; burn injury; acute cerebral hemorrhage or infarction; and any condition for which the patient requires assisted (e.g. mechanical) ventilation. It should be noted that, by their very nature, such conditions which require critical care are serious, severe, life-threatening forms of illness.

The terms “sample” or “biological sample” as used herein include any biological specimen obtained from a subject. Samples may include, without limitation, whole blood, plasma, serum, red blood cells, white blood cells (e.g., peripheral blood mononuclear cells), saliva, urine, stool (i.e., faeces), tears, sweat, sebum, nipple aspirate, ductal lavage, tumour exudates, synovial fluid, cerebrospinal fluid, lymph, fine needle aspirate, amniotic fluid, any other bodily fluid, cell lysates, cellular secretion products, inflammation fluid, semen and vaginal secretions. Preferred samples may include ones comprising any one or more markers as taught herein in detectable quantities. In preferred embodiments, the sample may be whole blood or a fractional component thereof such as, e.g., plasma, serum, or a cell pellet. Preferably the sample is readily obtainable by minimally invasive methods, allowing the removal or isolation of said sample from the subject. Samples may also include tissue samples and biopsies, tissue homogenates and the like.

Preferably, the sample used to detect the levels of any one or more markers as taught herein is blood plasma. The term “plasma” generally denotes the substantially colourless watery fluid of the blood that contains no cells, but in which the blood cells (erythrocytes, leukocytes, thrombocytes, etc.) are normally suspended, containing nutrients, sugars, proteins, minerals, enzymes, etc.

Equally preferred, the sample used to detect the levels of any one or more markers as taught herein is serum. The term “serum” refers to the component of blood that is neither a blood cell nor a clotting factor; the term refers to the blood plasma with the fibrinogens removed.

A molecule or analyte such as a protein, polypeptide or peptide, or a group of two or more molecules or analytes such as two or more proteins, polypeptides or peptides, is “measured” in a sample when the presence or absence and/or quantity of said molecule or analyte or of said group of molecules or analytes is detected or determined in the sample, preferably substantially to the exclusion of other molecules and analytes.

A parameter is “scored” or “measured” for or in a patient when the presence or absence and/or quantity of said parameter is detected or determined for or in the subject. For example, white blood cell count (expressed as the number of cells per litre) may be scored by counting the number of white blood cells in a sample. For example, a biophysical parameter (e.g., blood pressure) can be measured using standard tests and apparatus.

The terms “quantity”, “amount” and “level” are synonymous and generally well-understood in the art. The terms as used herein may particularly refer to an absolute quantification of a molecule or an analyte in a sample, or to a relative quantification of a molecule or analyte in a sample, i.e., relative to another value such as relative to a reference value as taught herein, or to a range of values indicating a base-line of the biomarker. These values or ranges may be obtained from a single patient or from a group of patients.

An absolute quantity of a molecule or analyte in a sample may be advantageously expressed as weight or as molar amount, or more commonly as a concentration, e.g., weight per volume or mol per volume.

A relative quantity of a molecule or analyte in a sample may be advantageously expressed as an increase or decrease or as a fold-increase or fold-decrease relative to said another value, such as relative to a reference value as taught herein. Performing a relative comparison between first and second parameters (e.g., first and second quantities) may but need not require determining first the absolute values of said first and second parameters. For example, a measurement method may produce quantifiable readouts (such as, e.g., signal intensities) for said first and second parameters, wherein said readouts are a function of the value of said parameters, and wherein said readouts may be directly compared to produce a relative value for the first parameter vs. the second parameter, without the actual need to first convert the readouts to absolute values of the respective parameters.

As used herein, the reference to any one marker (biomarker), nucleic acid, peptide, polypeptide or protein corresponds to the marker, nucleic acid, peptide, polypeptide or protein commonly known under the respective designations in the art. The terms encompass such markers, nucleic acids, proteins and polypeptides of any organism where found, and particularly of animals, preferably warm-blooded animals, more preferably vertebrates, yet more preferably mammals, including humans and non-human mammals, still more preferably of humans. The terms particularly encompass such markers, nucleic acids, proteins and polypeptides with a native sequence, i.e., ones of which the primary sequence is the same as that of the markers, nucleic acids, proteins and polypeptides found in or derived from nature. A skilled person understands that native sequences may differ between different species due to genetic divergence between such species. Moreover, native sequences may differ between or within different individuals of the same species due to normal genetic diversity (variation) within a given species. Also, native sequences may differ between or even within different individuals of the same species due to post-transcriptional or post-translational modifications. Any such variants or isoforms of markers, nucleic acids, proteins and polypeptides are intended herein. Accordingly, all sequences of markers, nucleic acids, proteins and polypeptides found in or derived from nature are considered “native”. The terms encompass the markers, nucleic acids, proteins and polypeptides when forming a part of a living organism, organ, tissue or cell, when forming a part of a biological sample, as well as when at least partly isolated from such sources. The terms also encompass proteins and polypeptides when produced by recombinant or synthetic means.

Exemplary human markers, nucleic acids, proteins or polypeptides as taught herein may be as annotated under NCBI Genbank (http://www.ncbi.nlm.nih.gov/) or Swissprot/Uniprot (http://www.uniprot.org/) accession numbers given below. A skilled person will also appreciate that in some instances said sequences may be of precursors (e.g., preproteins) of the markers, nucleic acids, proteins or polypeptides as taught herein and may include parts which are processed away from mature molecules. A skilled person will further appreciate that although only one or more isoforms may be listed below, all isoforms are intended. Unless otherwise specified, the entries in Table 1 are presented in the form: Name; Protein; Gene; Genbank RefSeq for one or more representative amino acid sequences (e.g., isoforms) followed by the Genbank sequence version, Genbank RefSeq for one or more representative mRNA sequences followed by the Genbank sequence version.

TABLE 1
Exemplary human markers

| | | | RefSeq | RefSeq |
|---|---|---|---|---|
| Name | SwissProt entry | Gene | Protein ID | mRNA ID |
| proteinase 3 (serine proteinase, neutrophil, Wegener | PRTN3_HUMAN | PRTN3 | NP_002768.3 | NM_002777.3 |
| granulomatosis autoantigen) | | | | |
| Macrophage mannose receptor 1 | MRC1_HUMAN | MRC1 | NP_002429.1 | NM_002438.2 |
| | | | | NM_001009567.1 |
| Pentraxin 3 | PTX3_HUMAN | PTX3 | NP_002843.2 | NM_002852.3 |
| interleukin 1 receptor, type II | IL1R2_HUMAN | IL1R2 | NP_004624.1 | NM_173343.1 |
| | | | NP_775465.1 | NM_004633.3 |
| exostoses (multiple) 2 | EXT2_HUMAN | EXT2 | NP_997005.1 | NM_207122.1 |
| | | | NP_000392.3 | NM_000401.3 |
| Mannosyl-oligosaccharide 1,2-alpha-mannosidase IA | MA1A1_HUMAN | MAN1A1 | NP_005898.2 | NM_005907.2 |
| Acyl-CoA-binding protein | ACBP_HUMAN | DBI | NP_001171514.1 | NM_001178043.1 |
| | | | NP_001073331.1 | NM_001079862.1 |
| Vesicular integral-membrane protein VIP36 | LMAN2_HUMAN | LMAN2 | NP_006807.1 | NM_006816.2 |
| Cystatin-C | CYTC_HUMAN | CST3 | NP_000090.1 | NM_000099.2 |
| C-reactive protein | CRP_HUMAN | CRP | NP_000558.2 | NM_000567.2 |
| Neuronal acetylcholine receptor subunit alpha-7 | ACHA7_HUMAN | CHRNA7 | NP_000737.1 | NM_000746.4 |
| | | | NP_683709.1 | |
| Cyclic AMP-dependent transcription factor ATF-6 alpha | ATF6A_HUMAN | ATF6 | NP_031374.2 | NM_007348.2 |
| Isoform Long of Beta-1,4-galactosyltransferase 1 | B4GT1_HUMAN | B4GALT1 | NP_001488.2 | NM_001497.3 |
| cathelicidin antimicrobial peptide | CAMP_HUMAN | CAMP | NP_004336.2 | NM_004345.3 |
| Golgi membrane protein 1 | GOLM1_HUMAN | GOLM1 | NP_808800.1 | NM_177937.2 |
| | | | NP_057632.2 | NM_016548.3 |
| Nidogen-1 | NID1_HUMAN | NID1 | NP_002499.2 | NM_002508.2 |
| matrix metallopeptidase 3 (stromelysin 1, progelatinase) | MMP3_HUMAN | MMP3 | NP_002413.1 | NM_002422.3 |
| Lipopolysaccharide-binding protein | LBP_HUMAN | LBP | NP_004130.2 | NM_004139.2 |
| Fibulin 1 | FBLN1_HUMAN | FBLN1 | NP_006478.2 | NM_006486.2 |
| | | | NP_006477.2 | |
| Polymeric-immunoglobulin receptor | PIGR_HUMAN | PIGR | NP_002635.2 | NM_002644.3 |
| TIMP Metalloproteinase inhibitor 1 | TIMP1_HUMAN | TIMP1 | NP_003245.1 | NM_003254.2 |
| glycosylphosphatidylinositol specific phospholipase D1 | PHLD_HUMAN | GPLD1 | NP_001494.2 | NM_001503.2 |
| Angiotensinogen | ANGT_HUMAN | AGT | NP_000020.1 | NM_000029.3 |
| Carboxypeptidase N catalytic chain | CBPN_HUMAN | CPN1 | NP_001299.1 | NM_001308.2 |
| Chitinase-3-like protein 1 | CH3L1_HUMAN | CHI3L1 | NP_001267.2 | NM_001276.2 |
| Macrophage colony-stimulating factor 1 | CSF1_HUMAN | CSF1 | NP_757351.1 | NM_000757.4 |
| | | | NP_000748.3 | NM_172212.2 |
| dystryglycan | DAG1_HUMAN | DAG1 | NP_001171107.1 | NM_001177639.1 |
| | | | NP_001159400.2 | NM_001177638.1 |
| | | | NP_001171106.1 | NM_001177635.1 |
| | | | NP_004384.4 | NM_001177634.1 |
| | | | NP_001171113.1 | NM_001177637.1 |
| | | | NP_001171112.1 | NM_001177636.1 |
| | | | NP_001171111.1 | NM_004393.4 |
| | | | NP_001171110.1 | NM_001165928.2 |
| | | | NP_001171105.1 | NM_001177644.1 |
| | | | NP_001171115.1 | NM_001177640.1 |
| | | | NP_001171114.1 | NM_001177641.1 |
| | | | NP_001171108.1 | NM_001177642.1 |
| | | | NP_001171109.1 | NM_001177643.1 |
| Fibrillin-1 | FBN1_HUMAN | FBN1 | NP_000129.3 | NM_000138.4 |
| Fibrinogen-like 1 | FGL1_HUMAN | FGL1 | NP_004458.3 | NM_201552.1 |
| | | | NP_671736.2 | NM_201553.1 |
| | | | NP_963847.1 | NM_147203.2 |
| | | | NP_963846.1 | NM_004467.3 |
| Glutathione synthetase | GSHB_HUMAN | GSS | NP_000169.1 | NM_000178.2 |
| Intercellular adhesion molecule 1 | ICAM1_HUMAN | ICAM1 | NP_000192.2 | NM_000201.2 |
| Lumican | LUM_HUMAN | LUM | NP_002336.1 | NM_002345.3 |
| S100 calcium binding protein A9 | S10A9_HUMAN | S100A9 | NP_002956.1 | NM_002965.3 |
| Serum amyloid A protein | SAA_HUMAN | SAA | NP_954630.1 | NM_030754.4 |
| | | | NP_000322.2 | NM_000331.4 |
| | | | NP_110381.2 | NM_199161.3 |
| | | | NP_001171477.1 | NM_001178006.1 |
| Serglycin | SRGN_HUMAN | SRGN | NP_002718.2 | NM_002727.2 |
| Vascular cell adhesion protein 1 | VCAM1_HUMAN | VCAM1 | NP_001069.1 | NM_001078.3 |
| calumenin | CALU_HUMAN | CALU | NP_001186600.1 | NM_001219.4 |
| | | | NP_001186603.1 | |
| | | | NP_001210.1 | |
| echinoderm microtubule associated protein like 3 | EMAL3_HUMAN | EML3 | NP_694997.2 | NM_153265.2 |
| Rho GDP dissociation inhibitor (GDI) beta | GDIR2_HUMAN | ARHGDIB | NP_001166.3 | NM_001175.4 |
| guanylate cyclase activator 2B (uroguanylin) | GUC2B_HUMAN | GUCA2B | NP_009033.1 | NM_007102.2 |
| heat shock 70 kDa protein 8 | HSP7C_HUMAN | HSPA8 | NP_006588.1 | NM_006597.3 |
| interleukin 13 receptor, alpha 1 | I13R1_HUMAN | IL13RA1 | NP_001551.1 | NM_001560.2 |
| moesin | MOES_HUMAN | MSN | NP_002435.1 | NM_002444.2 |
| Neutrophil gelatinase-associated lipocalin (lipocalin 2) | NGAL_HUMAN | NGAL | NP_005555.2 | NM_005564.3 |
| protein disulfide isomerase family A, member 6 | PDIA6_HUMAN | PDIA6 | NP_005733.1 | NM_005742.2 |
| proteasome (prosome, macropain) subunit, alpha type, 3 | PSA3_HUMAN | PSMA3 | NP_002779.1 | NM_152132.2 |
| | | | | NM_002788.3 |
| protein tyrosine phosphatase, receptor type, G | PTPRG_HUMAN | PTPRG | NP_002832.3 | NM_002841.3 |
| S100 calcium binding protein A8 | S10A8_HUMAN | S100A8 | NP_002955.2 | NM_002964.4 |
| cathepsin G | CATG_HUMAN | CTSG | NP_001902.1 | NM_001911.2 |
| neutrophil elastase | ELNE_HUMAN | ELANE | NP_001963.1 | NM_001972.2 |

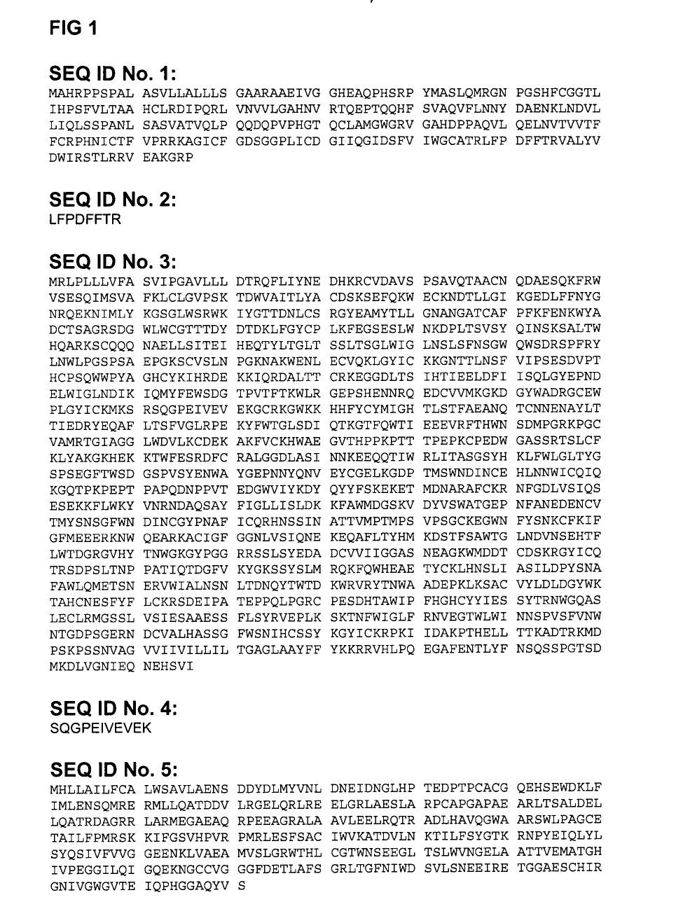

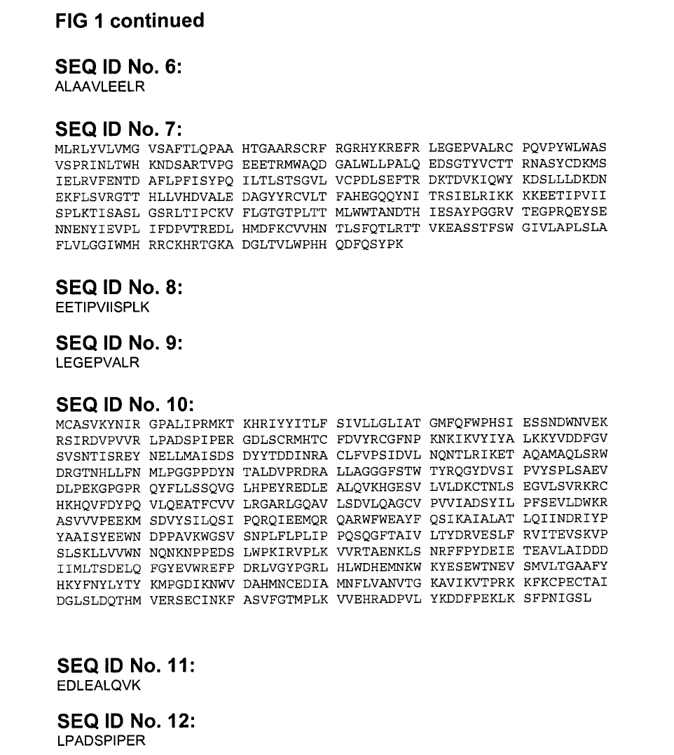

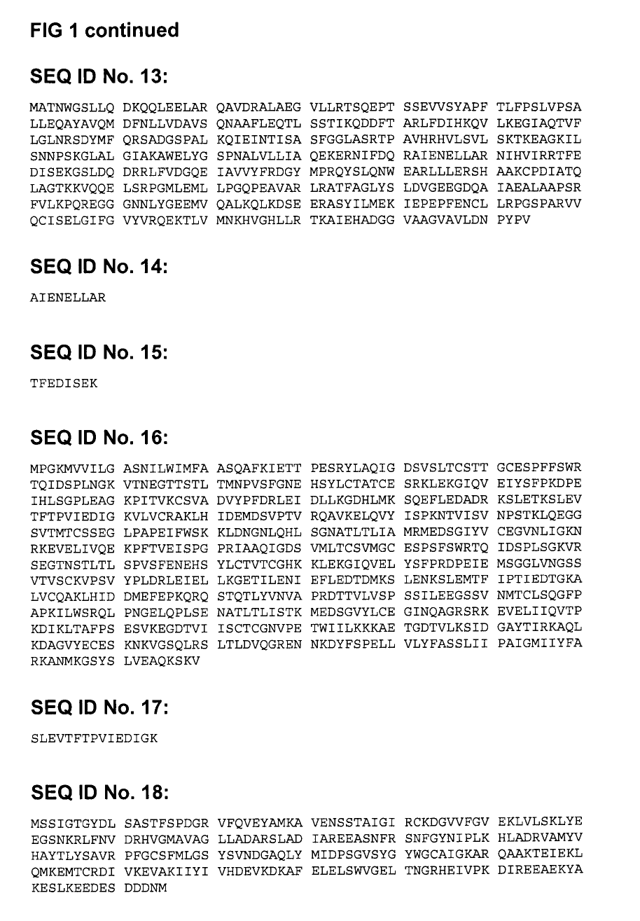

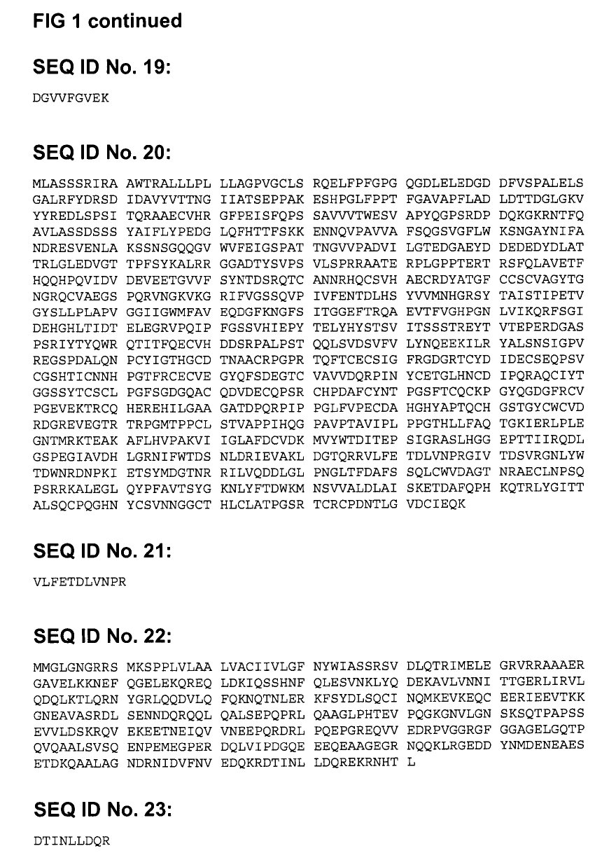

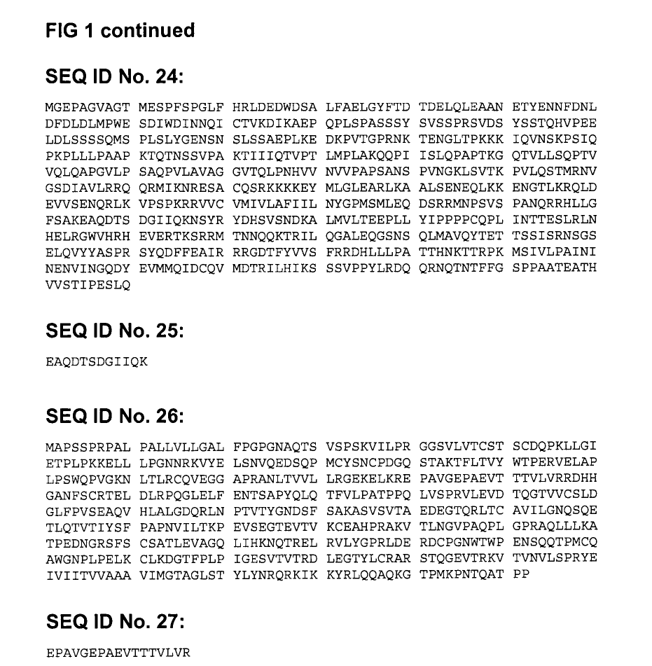

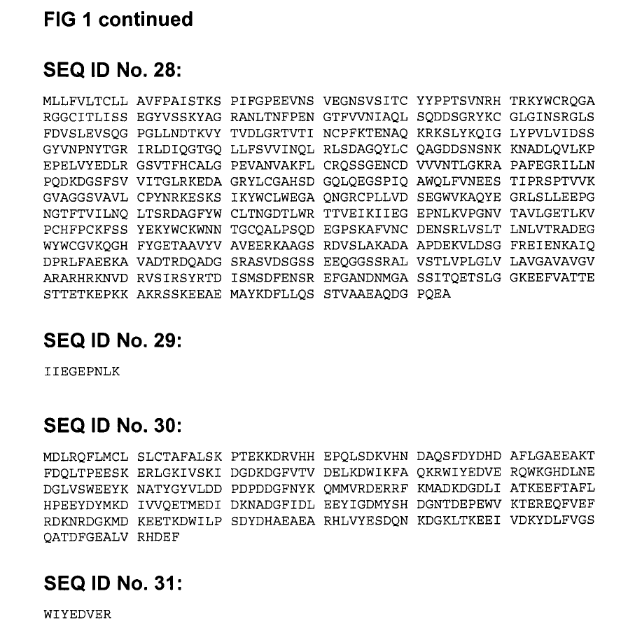

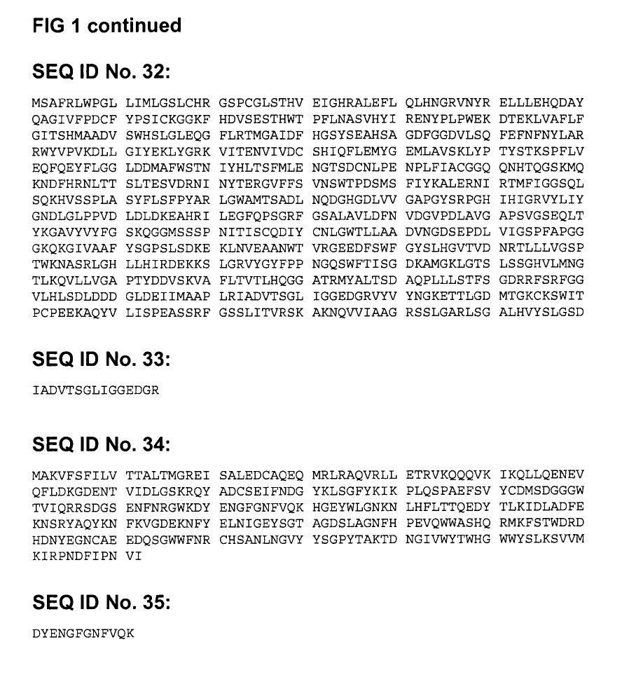

Exemplary proteinase 3 (PRTN3) includes, without limitation, human PRTN3 having primary amino acid sequence as annotated under NCBI Genbank accession number NP_{—}002768 (sequence version 3). For example, exemplary proteinase 3 (PRTN3) includes, without limitation, human PRTN3 having primary amino acid sequence as shown in FIG. 1 (SEQ ID No. 1). PRTN3 is one of three unique serine proteinases expressed by neutrophils.

PRTN3 has mostly been studied in relation to Wegener's granulomatosis, a systemic autoimmune disease characterized by necrotizing vasculitis and circulating antineutrophil cytoplasmic antibodies (ANCAs). These ANCAs are in patients with Wegener's granulomatosis mainly directed against proteinase 3 and, upon binding their target on the surface of neutrophils they induce neutrophil activation, respiratory burst and cytokine production (Preston et al., Cleve. Clin. J. Med., 2002, 69 Suppl 2: SII51-4). Serological measurement of anti-proteinase 3 auto-antibodies is part of the ANCA test to aid diagnosing autoimmune vasculitis.

Proteinase 3 is formed as an inactive pre-pro-enzyme with a leader signal peptide sequence that is cleaved leaving a pro-enzyme. Activation of the protease requires the removal of the pro-dipeptide sequence by cysteine protease dipeptidyl peptidase I (DPPI) (Adkison et al., J. Clin. Invest., 2002, 109(3): 363-71). In the circulation PRTN3 is rapidly inactivated by irreversible binding to SERPIN A1 (α₁-antitrypsin) (Baslund et al., J. Imunnol. Methods, 1994, 175(2): 215-25). Preferably, to measure PRTN3, an assay may be chosen to detect the active chain of the enzyme. Accordingly, in the experimental section, a peptide as set forth in SEQ ID NO: 2 (FIG. 1) from the active chain of the enzyme was used to measure PRTN3.

Exemplary macrophage mannose receptor 1 (MRC1) includes, without limitation, human MRC1 having primary amino acid sequence as annotated under NCBI Genbank accession number NP_{—}002429 (sequence version 1). For example, exemplary mannose receptor 1 (MRC1) includes, without limitation, human MRC1 having primary amino acid sequence as shown in FIG. 1 (SEQ ID No. 3). The peptide targeted in MRC1 in the MASSterclass® assays used in the experimental section is given in SEQ ID NO: 4 (FIG. 1).

Exemplary pentraxin-3 (PTX3) includes, without limitation, human PTX3 having primary amino acid sequence as annotated under NCBI Genbank accession number NP_{—}002843 (sequence version 2). For example, pentraxin-3 (PTX3) includes, without limitation, human PTX3 having primary amino acid sequence as shown in FIG. 1 (SEQ ID No. 5). The peptide targeted in PTX3 in the MASSterclass® assays used in the experimental section is given in SEQ ID NO: 6 (FIG. 1).

Exemplary interleukine 1 receptor type II (IL1R2) includes, without limitation, human IL1R2 having primary amino acid sequence as annotated under NCBI Genbank accession number NP_{—}004624 (sequence version 1). For example, interleukine 1 receptor type II (IL1R2) includes, without limitation, human IL1R2 having primary amino acid sequence as shown in FIG. 1 (SEQ ID No. 7). The peptides targeted in IL1R2 in the MASSterclass® assays used in the experimental section are given in SEQ ID NO: 8 and 9 (FIG. 1).

Exemplary exostoses 2 (EXT2) includes, without limitation, human EXT2 having primary amino acid sequence as annotated under NCBI Genbank accession number NP_{—}997005 (sequence version 1).

For example, exostoses 2 (EXT2) includes, without limitation, human EXT2 having primary amino acid sequence as shown in FIG. 1 (SEQ ID No. 10). The peptides targeted in EXT2 in the MASSterclass® assays used in the experimental section are given in SEQ ID NO: 11 and 12 (FIG. 1).

The reference herein to any biomarker, nucleic acid, protein or polypeptide may also encompass fragments thereof. Hence, the reference herein to measuring (or measuring the quantity of) any one biomarker, nucleic acid, protein or polypeptide may encompass measuring the biomarker, nucleic acid, protein or polypeptide, such as, e.g., measuring the mature and/or the processed soluble/secreted form (e.g. plasma circulating form) thereof and/or measuring one or more fragments thereof.

For example, any biomarker, nucleic acid, protein or polypeptide and/or one or more fragments thereof may be measured collectively, such that the measured quantity corresponds to the sum amounts of the collectively measured species. In another example, any biomarker, nucleic acid, protein or polypeptide and/or one or more fragments thereof may be measured each individually. Preferably, said fragment may be a plasma circulating (i.e., not cell- or membrane-bound) form. Without being bound by any theory, such circulating forms may be derived from full-length biomarkers, nucleic acids, proteins or polypeptides through natural processing, or may be resulting from known degradation processes occurring in a sample. In certain situations, the circulating form may also be the full-length biomarker, nucleic acid, protein or polypeptide, which is found to be circulating in the plasma. Said “circulating form” may thus be any biomarker, nucleic acid, protein or polypeptide or any processed soluble form thereof or fragments of either one, that is circulating in the sample, i.e. which is not bound to a cell- or membrane fraction of said sample.

Unless otherwise apparent from the context, reference herein to any biomarker, nucleic acid, protein or polypeptide and fragments thereof may generally also encompass modified forms of said biomarker, nucleic acid, protein or polypeptide and fragments such as bearing post-expression modifications including, for example, phosphorylation, glycosylation, lipidation, methylation, cysteinylation, sulphonation, glutathionylation, acetylation, oxidation of methionine to methionine sulphoxide or methionine sulphone, and the like.

In an embodiment, any biomarker, nucleic acid, protein or polypeptide and fragments thereof may be human, i.e., their primary sequence may be the same as a corresponding primary sequence of or present in a naturally occurring human biomarker, nucleic acid, protein or polypeptide. Hence, the qualifier “human” in this connection relates to the primary sequence of the respective biomarker, nucleic acid, protein or polypeptide, rather than to its origin or source. For example, such biomarker, nucleic acid, protein or polypeptide and fragments may be present in or isolated from samples of human subjects or may be obtained by other means (e.g., by recombinant expression, cell-free translation or non-biological peptide synthesis).

The term “fragment” of a protein, polypeptide or peptide generally refers to N-terminally and/or C-terminally deleted or truncated forms of said protein, polypeptide or peptide. The term encompasses fragments arising by any mechanism, such as, without limitation, by alternative translation, exo- and/or endo-proteolysis and/or degradation of said peptide, polypeptide or protein, such as, for example, in vivo or in vitro, such as, for example, by physical, chemical and/or enzymatic proteolysis. Without limitation, a fragment of a protein, polypeptide or peptide may represent at least about 5%, or at least about 10%, e.g., ≧20%, ≧30% or ≧40%, such as ≧50%, e.g., ≧60%, ≧70% or ≧80%, or even ≧90% or ≧95% of the amino acid sequence of said protein, polypeptide or peptide.

For example, a fragment may include a sequence of ≧5 consecutive amino acids, or ≧10 consecutive amino acids, or ≧20 consecutive amino acids, or ≧30 consecutive amino acids, e.g., ≧40 consecutive amino acids, such as for example ≧50 consecutive amino acids, e.g., ≧60, ≧70, ≧80, ≧90, ≧100, ≧200, ≧300, ≧400, ≧500 or ≧600 consecutive amino acids of the corresponding full length protein.

In an embodiment, a fragment may be N-terminally and/or C-terminally truncated by between 1 and about 20 amino acids, such as, e.g., by between 1 and about 15 amino acids, or by between 1 and about 10 amino acids, or by between 1 and about 5 amino acids, compared to the corresponding mature, full-length protein or its soluble or plasma circulating form.

In an embodiment, fragments of a given protein, polypeptide or peptide may be achieved by in vitro proteolysis of said protein, polypeptide or peptide to obtain advantageously detectable peptide(s) from a sample. For example, such proteolysis may be effected by suitable physical, chemical and/or enzymatic agents, e.g., proteinases, preferably endoproteinases, i.e., protease cleaving internally within a protein, polypeptide or peptide chain. A non-limiting list of suitable endoproteinases includes serine proteinases (EC 3.4.21), threonine proteinases (EC 3.4.25), cysteine proteinases (EC 3.4.22), aspartic acid proteinases (EC 3.4.23), metalloproteinases (EC 3.4.24) and glutamic acid proteinases. Exemplary non-limiting endoproteinases include trypsin, chymotrypsin, elastase, Lysobacter enzymogenes endoproteinase Lys-C, Staphylococcus aureus endoproteinase Glu-C (endopeptidase V8) or Clostridium histolyticum endoproteinase Arg-C (clostripain). Further known or yet to be identified enzymes may be used; a skilled person will be able to choose suitable protease(s) on the basis of their cleavage specificity and frequency to achieve desired peptide forms. Preferably, the proteolysis may be effected by endopeptidases of the trypsin type (EC 3.4.21.4), preferably trypsin, such as, without limitation, preparations of trypsin from bovine pancreas, human pancreas, porcine pancreas, recombinant trypsin, Lys-acetylated trypsin, trypsin in solution, trypsin immobilised to a solid support, etc. Trypsin is particularly useful, inter alia due to high specificity and efficiency of cleavage. The invention also contemplates the use of any trypsin-like protease, i.e., with a similar specificity to that of trypsin. Otherwise, chemical reagents may be used for proteolysis. For example, CNBr can cleave at Met; BNPS-skatole can cleave at Trp. The conditions for treatment, e.g., protein concentration, enzyme or chemical reagent concentration, pH, buffer, temperature, time, can be determined by the skilled person depending on the enzyme or chemical reagent employed.

The term “isolated” with reference to a particular component (such as for instance, nucleic acid, protein, polypeptide, peptide or fragment thereof) generally denotes that such component exists in separation from—for example, has been separated from or prepared in separation from—one or more other components of its natural environment. For instance, an isolated human or animal nucleic acid, protein, polypeptide, peptide or fragment exists in separation from a human or animal body where it occurs naturally.

The term “isolated” as used herein may preferably also encompass the qualifier “purified”. As used herein, the term “purified” with reference to nucleic acid(s), protein(s), polypeptide(s), peptide(s) and/or fragment(s) thereof does not require absolute purity. Instead, it denotes that such nucleic acid(s), protein(s), polypeptide(s), peptide(s) and/or fragment(s) is (are) in a discrete environment in which their abundance (conveniently expressed in terms of mass or weight or concentration) relative to other proteins is greater than in a biological sample. A discrete environment denotes a single medium, such as for example a single solution, gel, precipitate, lyophilisate, etc. Purified nucleic acids, peptides, polypeptides or fragments may be obtained by known methods including, for example, laboratory or recombinant synthesis, chromatography, preparative electrophoresis, centrifugation, precipitation, affinity purification, etc.

Purified protein(s), polypeptide(s), peptide(s) and/or fragment(s) may preferably constitute by weight ≧10%, more preferably ≧50%, such as ≧60%, yet more preferably ≧70%, such as ≧80%, and still more preferably ≧90%, such as ≧95%, ≧96%, ≧97%, ≧98%, ≧99% or even 100%, of the protein content of the discrete environment. Protein content may be determined, e.g., by the Lowry method (Lowry et al. 1951. J Biol Chem 193: 265), optionally as described by Hartree 1972 (Anal Biochem 48: 422-427). Also, purity of peptides or polypeptides may be determined by SDS-PAGE under reducing or non-reducing conditions using Coomassie blue or, preferably, silver stain.

In some embodiments, reagents disclosed herein may comprise a detectable label. The term “label” refers to any atom, molecule, moiety or biomolecule that may be used to provide a detectable and preferably quantifiable read-out or property, and that may be attached to or made part of an entity of interest, such as a peptide or polypeptide or a specific-binding agent. Labels may be suitably detectable by mass spectrometric, spectroscopic, optical, colourimetric, magnetic, photochemical, biochemical, immunochemical or chemical means. Labels include without limitation dyes; radiolabels such as ³²P, ³³P, ³⁵S, ¹²⁵I, ¹³¹I; electron-dense reagents; enzymes (e.g. horse-radish phosphatise or alkaline phosphatise as commonly used in immunoassays); binding moieties such as biotin-streptavidin; haptens such as digoxigenin; luminogenic, phosphorescent or fluorogenic moieties; mass tags; and fluorescent dyes alone or in combination with moieties that may suppress or shift emission spectra by fluorescence resonance energy transfer (FRET).

For example, the label may be a mass-altering label. Preferably, a mass-altering label may involve the presence of a distinct stable isotope in one or more amino acids of the peptide vis-à-vis its corresponding non-labelled peptide. Mass-labelled peptides are particularly useful as positive controls, standards and calibrators in mass spectrometry applications. In particular, peptides including one or more distinct isotopes are chemically alike, separate chromatographically and electrophoretically in the same manner and also ionise and fragment in the same way. However, in a suitable mass analyser such peptides and optionally select fragmentation ions thereof will display distinguishable m/z ratios and may thus be discriminated. Examples of pairs of distinguishable stable isotopes include H and D, ¹²C and ¹³C, ¹⁴N and ¹⁵N or ¹⁶O and ¹⁸O. Usually, peptides and proteins of biological samples analysed in the present invention may substantially only contain common isotopes having high prevalence in nature, such as for example H, ¹²C, ¹⁴N and a ¹⁶O. In such case, the mass-labelled peptide may be labelled with one or more uncommon isotopes having low prevalence in nature, such as for instance D, ¹³C, ¹⁵N and/or ¹⁸O. It is also conceivable that in cases where the peptides or proteins of a biological sample would include one or more uncommon isotopes, the mass-labelled peptide may comprise the respective common isotope(s).

Isotopically-labelled synthetic peptides may be obtained inter alia by synthesising or recombinantly producing such peptides using one or more isotopically-labelled amino acid substrates, or by chemically or enzymatically modifying unlabelled peptides to introduce thereto one or more distinct isotopes. By means of example and not limitation, D-labelled peptides may be synthesised or recombinantly produced in the presence of commercially available deuterated L-methionine CH₃—S-CD₂CD₂-CH(NH₂)—COOH or deuterated arginine H₂NC(═NH)—NH—(CD₂)₃-CD(NH₂)—COOH. It shall be appreciated that any amino acid of which deuterated or ¹⁵N- or ¹³C-containing forms exist may be considered for synthesis or recombinant production of labelled peptides. In another non-limiting example, a peptide may be treated with trypsin in H₂¹⁶O or H₂¹⁸O, leading to incorporation of two oxygens (¹⁶O or ¹⁸O, respectively) at the COOH-termini of said peptide (e.g., US 2006/105415).

Also contemplated is the use of biomarkers, peptides, polypeptides or proteins and fragments thereof as taught herein, optionally comprising a detectable label, as (positive) controls, standards or calibators in qualitative or quantitative detection assays (measurement methods) of said biomarkers, peptides, polypeptides or proteins and fragments thereof, and particularly in such methods for the diagnosis, prediction, prognosis and/or monitoring the diseases or conditions as taught herein in subjects. The biomarkers, proteins, polypeptides or peptides may be supplied in any form, inter alia as precipitate, vacuum-dried, lyophilisate, in solution as liquid or frozen, or covalently or non-covalently immobilised on solid phase, such as for example, on solid chromatographic matrix or on glass or plastic or other suitable surfaces (e.g., as a part of peptide arrays and microarrays). The peptides may be readily prepared, for example, isolated from natural sources, or prepared recombinantly or synthetically.

Further disclosed are binding agents capable of specifically binding to biomarkers, peptides, polypeptides or proteins and fragments thereof as taught herein. Binding agents as intended throughout this specification may include inter alia an antibody, aptamer, photoaptamer, protein, peptide, peptidomimetic or a small molecule.

The term “specifically bind” as used throughout this specification means that an agent (denoted herein also as “specific-binding agent”) binds to one or more desired molecules or analytes substantially to the exclusion of other molecules which are random or unrelated, and optionally substantially to the exclusion of other molecules that are structurally related. The term “specifically bind” does not necessarily require that an agent binds exclusively to its intended target(s). For example, an agent may be said to specifically bind to target(s) of interest if its affinity for such intended target(s) under the conditions of binding is at least about 2-fold greater, preferably at least about 5-fold greater, more preferably at least about 10-fold greater, yet more preferably at least about 25-fold greater, still more preferably at least about 50-fold greater, and even more preferably at least about 100-fold or more greater, than its affinity for a non-target molecule.

Specific binding agents as used throughout this specification may include inter alia an antibody, aptamer, spiegelmer (L-aptamer), photoaptamer, protein, peptide, peptidomimetic or a small molecule.

Preferably, the agent may bind to its intended target(s) with affinity constant (K_{A}) of such binding K_{A}≧1×10⁶ M⁻¹, more preferably K_{A}≧1×10⁷ M⁻¹, yet more preferably K_{A}≧1×10⁸ M⁻¹, even more preferably K_{A}≧1×10⁹ M⁻¹, and still more preferably K_{A}≧1×10¹⁰ M⁻¹ or K_{A}≧1×10¹¹ M⁻¹, wherein K_{A}=[SBA_T]/[SBA][T], SBA denotes the specific-binding agent, T denotes the intended target. Determination of K_{A} can be carried out by methods known in the art, such as for example, using equilibrium dialysis and Scatchard plot analysis.

As used herein, the term “antibody” is used in its broadest sense and generally refers to any immunologic binding agent. The term specifically encompasses intact monoclonal antibodies, polyclonal antibodies, multivalent (e.g., 2-, 3- or more-valent) and/or multi-specific antibodies (e.g., bi- or more-specific antibodies) formed from at least two intact antibodies, and antibody fragments insofar they exhibit the desired biological activity (particularly, ability to specifically bind an antigen of interest), as well as multivalent and/or multi-specific composites of such fragments. The term “antibody” is not only inclusive of antibodies generated by methods comprising immunisation, but also includes any polypeptide, e.g., a recombinantly expressed polypeptide, which is made to encompass at least one complementarity-determining region (CDR) capable of specifically binding to an epitope on an antigen of interest. Hence, the term applies to such molecules regardless whether they are produced in vitro or in vivo.

An antibody may be any of IgA, IgD, IgE, IgG and IgM classes, and preferably IgG class antibody. An antibody may be a polyclonal antibody, e.g., an antiserum or immunoglobulins purified there from (e.g., affinity-purified). An antibody may be a monoclonal antibody or a mixture of monoclonal antibodies. Monoclonal antibodies can target a particular antigen or a particular epitope within an antigen with greater selectivity and reproducibility. By means of example and not limitation, monoclonal antibodies may be made by the hybridoma method first described by Kohler et al. 1975 (Nature 256: 495), or may be made by recombinant DNA methods (e.g., as in U.S. Pat. No. 4,816,567). Monoclonal antibodies may also be isolated from phage antibody libraries using techniques as described by Clackson et al. 1991 (Nature 352: 624-628) and Marks et al. 1991 (J Mol Biol 222: 581-597), for example.

Antibody binding agents may be antibody fragments. “Antibody fragments” comprise a portion of an intact antibody, comprising the antigen-binding or variable region thereof. Examples of antibody fragments include Fab, Fab′, F(ab′)2, Fv and scFv fragments; diabodies; linear antibodies; single-chain antibody molecules; and multivalent and/or multispecific antibodies formed from antibody fragment(s), e.g., dibodies, tribodies, and multibodies. The above designations Fab, Fab′, F(ab′)2, Fv, scFv etc. are intended to have their art-established meaning.

The term antibody includes antibodies originating from or comprising one or more portions derived from any animal species, preferably vertebrate species, including, e.g., birds and mammals. Without limitation, the antibodies may be chicken, turkey, goose, duck, guinea fowl, quail or pheasant. Also without limitation, the antibodies may be human, murine (e.g., mouse, rat, etc.), donkey, rabbit, goat, sheep, guinea pig, camel (e.g., Camelus bactrianus and Camelus dromaderius), llama (e.g., Lama paccos, Lama glama or Lama vicugna) or horse.

A skilled person will understand that an antibody can include one or more amino acid deletions, additions and/or substitutions (e.g., conservative substitutions), insofar such alterations preserve its binding of the respective antigen. An antibody may also include one or more native or artificial modifications of its constituent amino acid residues (e.g., glycosylation, etc.).

Methods of producing polyclonal and monoclonal antibodies as well as fragments thereof are well known in the art, as are methods to produce recombinant antibodies or fragments thereof (see for example, Harlow and Lane, “Antibodies: A Laboratory Manual”, Cold Spring Harbour Laboratory, New York, 1988; Harlow and Lane, “Using Antibodies: A Laboratory Manual”, Cold Spring Harbour Laboratory, New York, 1999, ISBN 0879695447; “Monoclonal Antibodies: A Manual of Techniques”, by Zola, ed., CRC Press 1987, ISBN 0849364760; “Monoclonal Antibodies: A Practical Approach”, by Dean & Shepherd, eds., Oxford University Press 2000, ISBN 0199637229; Methods in Molecular Biology, vol. 248: “Antibody Engineering: Methods and Protocols”, Lo, ed., Humana Press 2004, ISBN 1588290921).

The term “aptamer” refers to single-stranded or double-stranded oligo-DNA, oligo-RNA or oligo-DNA/RNA or any analogue thereof that specifically binds to a target molecule such as a peptide. Advantageously, aptamers display fairly high specificity and affinity (e.g., K_{A} in the order 1×10⁹ M⁻¹) for their targets. Aptamer production is described inter alia in U.S. Pat. No. 5,270,163; Ellington & Szostak 1990 (Nature 346: 818-822); Tuerk & Gold 1990 (Science 249: 505-510); or “The Aptamer Handbook: Functional Oligonucleotides and Their Applications”, by Klussmann, ed., Wiley-VCH 2006, ISBN 3527310592, incorporated by reference herein. The term “photoaptamer” refers to an aptamer that contains one or more photoreactive functional groups that can covalently bind to or crosslink with a target molecule. The term “peptidomimetic” refers to a non-peptide agent that is a topological analogue of a corresponding peptide. Methods of rationally designing peptidomimetics of peptides are known in the art. For example, the rational design of three peptidomimetics based on the sulphated 8-mer peptide CCK26-33, and of two peptidomimetics based on the 11-mer peptide Substance P, and related peptidomimetic design principles, are described in Horwell 1995 (Trends Biotechnol 13: 132-134).

The term “small molecule” refers to compounds, preferably organic compounds, with a size comparable to those organic molecules generally used in pharmaceuticals. The term excludes biological macromolecules (e.g., proteins, nucleic acids, etc.). Preferred small organic molecules range in size up to about 5000 Da, e.g., up to about 4000, preferably up to 3000 Da, more preferably up to 2000 Da, even more preferably up to about 1000 Da, e.g., up to about 900, 800, 700, 600 or up to about 500 Da.

Hence, also disclosed are methods for immunising animals, e.g., non-human animals such as laboratory or farm, animals using (i.e., using as the immunising antigen) any one or more (isolated) markers, peptides, polypeptides or proteins and fragments thereof as taught herein, optionally attached to a presenting carrier. Immunisation and preparation of antibody reagents from immune sera is well-known per se and described in documents referred to elsewhere in this specification. The animals to be immunised may include any animal species, preferably warm-blooded species, more preferably vertebrate species, including, e.g., birds, fish, and mammals. Without limitation, the antibodies may be chicken, turkey, goose, duck, guinea fowl, shark, quail or pheasant. Also without limitation, the antibodies may be human, murine (e.g., mouse, rat, etc.), donkey, rabbit, goat, sheep, guinea pig, shark, camel, llama or horse. The term “presenting carrier” or “carrier” generally denotes an immunogenic molecule which, when bound to a second molecule, augments immune responses to the latter, usually through the provision of additional T cell epitopes. The presenting carrier may be a (poly)peptidic structure or a non-peptidic structure, such as inter alia glycans, polyethylene glycols, peptide mimetics, synthetic polymers, etc. Exemplary non-limiting carriers include human Hepatitis B virus core protein, multiple C3d domains, tetanus toxin fragment C or yeast Ty particles.

Immune sera obtained or obtainable by immunisation as taught herein may be particularly useful for generating antibody reagents that specifically bind to any one or more biomarkers, peptides, polypeptides or proteins and fragments thereof disclosed herein.

The binding molecule may labelled with a tag that permits detection with another agent (e.g. with a probe binding partner). Such tags may be, for example, biotin, streptavidin, his-tag, myc tag, maltose, maltose binding protein or any other kind of tag known in the art that has a binding partner. Example of associations which may be utilised in the probe:binding partner arrangement may be any, and includes, for example biotin:streptavidin, his-tag:metal ion (e.g. Ni²⁺), maltose:maltose binding protein.

The binding molecule conjugate may be associated with or attached to a detection agent to facilitate detection. Examples of lab detection agents include, but are not limited to, luminescent labels; colourimetric labels, such as dyes; fluorescent labels; or chemical labels, such as electroactive agents (e.g., ferrocyanide); enzymes; radioactive labels; or radiofrequency labels. More commonly, the detection agent is a particle. Examples of particles useful in the practice of the invention include, but are not limited to, colloidal gold particles; colloidal sulphur particles; colloidal selenium particles; colloidal barium sulfate particles; colloidal iron sulfate particles; metal iodate particles; silver halide particles; silica particles; colloidal metal (hydrous) oxide particles; colloidal metal sulfide particles; colloidal lead selenide particles; colloidal cadmium selenide particles; colloidal metal phosphate particles; colloidal metal ferrite particles; any of the above-mentioned colloidal particles coated with organic or inorganic layers; protein or peptide molecules; liposomes; or organic polymer latex particles, such as polystyrene latex beads. Preferable particles are colloidal gold particles. Colloidal gold may be made by any conventional means, such as the methods outlined in G. Frens, 1973 Nature Physical Science, 241:20 (1973). Alternative methods may be described in U.S. Pat. Nos. 5,578,577, 5,141,850; 4,775,636; 4,853,335; 4,859,612; 5,079,172; 5,202,267; 5,514,602; 5,616,467; 5,681,775.

Any existing, available or conventional separation, detection and quantification methods may be used herein to measure the presence or absence (e.g., readout being present vs. absent; or detectable amount vs. undetectable amount) and/or quantity (e.g., readout being an absolute or relative quantity, such as, for example, absolute or relative concentration) of biomarkers, peptides, polypeptides, proteins and/or fragments thereof in samples (any molecules or analytes of interest to be so-measured in samples, including any one or more biomarkers, peptides, polypeptides, proteins and fragments thereof as taught herein, may be herein below referred to collectively as biomarkers).

For example, such methods may include biochemical assay methods, immunoassay methods, mass spectrometry analysis methods, or chromatography methods, or combinations thereof.

The term “immunoassay” generally refers to methods known as such for detecting one or more molecules or analytes of interest in a sample, wherein specificity of an immunoassay for the molecule(s) or analyte(s) of interest is conferred by specific binding between a specific-binding agent, commonly an antibody, and the molecule(s) or analyte(s) of interest. Immunoassay technologies include without limitation direct ELISA (enzyme-linked immunosorbent assay), indirect ELISA, sandwich ELISA, competitive ELISA, multiplex ELISA, radioimmunoassay (RIA), ELISPOT technologies, and other similar techniques known in the art. Principles of these immunoassay methods are known in the art, for example John R. Crowther, “The ELISA Guidebook”, 1st ed., Humana Press 2000, ISBN 0896037282.

By means of further explanation and not limitation, direct ELISA employs a labelled primary antibody to bind to and thereby quantify target antigen in a sample immobilised on a solid support such as a microwell plate. Indirect ELISA uses a non-labelled primary antibody which binds to the target antigen and a secondary labelled antibody that recognises and allows the quantification of the antigen-bound primary antibody. In sandwich ELISA the target antigen is captured from a sample using an immobilised ‘capture’ antibody which binds to one antigenic site within the antigen, and subsequent to removal of non-bound analytes the so-captured antigen is detected using a ‘detection’ antibody which binds to another antigenic site within said antigen, where the detection antibody may be directly labelled or indirectly detectable as above. Competitive ELISA uses a labelled ‘competitor’ that may either be the primary antibody or the target antigen. In an example, non-labelled immobilised primary antibody is incubated with a sample, this reaction is allowed to reach equilibrium, and then labelled target antigen is added. The latter will bind to the primary antibody wherever its binding sites are not yet occupied by non-labelled target antigen from the sample. Thus, the detected amount of bound labelled antigen inversely correlates with the amount of non-labelled antigen in the sample. Multiplex ELISA allows simultaneous detection of two or more analytes within a single compartment (e.g., microplate well) usually at a plurality of array addresses (see, for example, Nielsen & Geierstanger 2004. J Immunol Methods 290: 107-20 and Ling et al. 2007. Expert Rev Mol Diagn 7: 87-98 for further guidance). As appreciated, labelling in ELISA technologies is usually by enzyme (such as, e.g., horse-radish peroxidase) conjugation and the end-point is typically colourimetric, chemiluminescent or fluorescent, magnetic, piezo electric, pyroelectric and other.

Radioimmunoassay (RIA) is a competition-based technique and involves mixing known quantities of radioactively-labelled (e.g., ¹²⁵I- or ¹³¹I-labelled) target antigen with antibody to said antigen, then adding non-labelled or ‘cold’ antigen from a sample and measuring the amount of labelled antigen displaced (see, e.g., “An Introduction to Radioimmunoassay and Related Techniques”, by Chard T, ed., Elsevier Science 1995, ISBN 0444821198 for guidance).

Generally, any mass spectrometric (MS) techniques that are capable of obtaining precise information on the mass of peptides, and preferably also on fragmentation and/or (partial) amino acid sequence of selected peptides (e.g., in tandem mass spectrometry, MS/MS; or in post source decay, TOF MS), are useful herein. Suitable peptide MS and MS/MS techniques and systems are well-known per se (see, e.g., Methods in Molecular Biology, vol. 146: “Mass Spectrometry of Proteins and Peptides”, by Chapman, ed., Humana Press 2000, ISBN 089603609x; Biemann 1990. Methods Enzymol 193: 455-79; or Methods in Enzymology, vol. 402: “Biological Mass Spectrometry”, by Burlingame, ed., Academic Press 2005, ISBN 9780121828073) and may be used herein. MS arrangements, instruments and systems suitable for biomarker peptide analysis may include, without limitation, matrix-assisted laser desorption/ionisation time-of-flight (MALDI-TOF) MS; MALDI-TOF post-source-decay (PSD); MALDI-TOF/TOF; surface-enhanced laser desorption/ionization time-of-flight mass spectrometry (SELDI-TOF) MS; electrospray ionization mass spectrometry (ESI-MS); ESI-MS/MS; ESI-MS/(MS)ⁿ (n is an integer greater than zero); ESI 3D or linear (2D) ion trap MS; ESI triple quadrupole MS; ESI quadrupole orthogonal TOF (Q-TOF); ESI Fourier transform MS systems; desorption/ionization on silicon (DIOS); secondary ion mass spectrometry (SIMS); atmospheric pressure chemical ionization mass spectrometry (APCI-MS); APCI-MS/MS; APCI-(MS)ⁿ; atmospheric pressure photoionization mass spectrometry (APPI-MS); APPI-MS/MS; and APPI-(MS)ⁿ. Peptide ion fragmentation in tandem MS (MS/MS) arrangements may be achieved using manners established in the art, such as, e.g., collision induced dissociation (CID). Detection and quantification of biomarkers by mass spectrometry may involve multiple reaction monitoring (MRM), such as described among others by Kuhn et al. 2004 (Proteomics 4: 1175-86). MS peptide analysis methods may be advantageously combined with upstream peptide or protein separation or fractionation methods, such as for example with the chromatographic and other methods described herein below.

Chromatography may also be used for measuring biomarkers. As used herein, the term “chromatography” encompasses methods for separating chemical substances, referred to as such and vastly available in the art. In a preferred approach, chromatography refers to a process in which a mixture of chemical substances (analytes) carried by a moving stream of liquid or gas (“mobile phase”) is separated into components as a result of differential distribution of the analytes, as they flow around or over a stationary liquid or solid phase (“stationary phase”), between said mobile phase and said stationary phase. The stationary phase may be usually a finely divided solid, a sheet of filter material, or a thin film of a liquid on the surface of a solid, or the like. Chromatography is also widely applicable for the separation of chemical compounds of biological origin, such as, e.g., amino acids, proteins, fragments of proteins or peptides, etc.

Chromatography as used herein may be preferably columnar (i.e., wherein the stationary phase is deposited or packed in a column), preferably liquid chromatography, and yet more preferably HPLC. While particulars of chromatography are well known in the art, for further guidance see, e.g., Meyer M., 1998, ISBN: 047198373X, and “Practical HPLC Methodology and Applications”, Bidlingmeyer, B. A., John Wiley & Sons Inc., 1993. Exemplary types of chromatography include, without limitation, high-performance liquid chromatography (HPLC), normal phase HPLC (NP-HPLC), reversed phase HPLC (RP-HPLC), ion exchange chromatography (IEC), such as cation or anion exchange chromatography, hydrophilic interaction chromatography (HILIC), hydrophobic interaction chromatography (HIC), size exclusion chromatography (SEC) including gel filtration chromatography or gel permeation chromatography, chromatofocusing, affinity chromatography such as immuno-affinity, immobilised metal affinity chromatography, and the like.

Chromatography, including single-, two- or more-dimensional chromatography, may be used as a peptide fractionation method in conjunction with a further peptide analysis method, such as for example, with a downstream mass spectrometry analysis as described elsewhere in this specification.

Further peptide or polypeptide separation, identification or quantification methods may be used, optionally in conjunction with any of the above described analysis methods, for measuring biomarkers in the present disclosure. Such methods include, without limitation, chemical extraction partitioning, isoelectric focusing (IEF) including capillary isoelectric focusing (CIEF), capillary isotachophoresis (CITP), capillary electrochromatography (CEC), and the like, one-dimensional polyacrylamide gel electrophoresis (PAGE), two-dimensional polyacrylamide gel electrophoresis (2D-PAGE), capillary gel electrophoresis (CGE), capillary zone electrophoresis (CZE), micellar electrokinetic chromatography (MEKC), free flow electrophoresis (FFE), etc.

The level of biomarkers at the RNA level may be established using RNA analysis of placental tissue obtained e.g. using transcervical placental biopsy during early pregnancy or similar methods not endangering the pregnancy. This test involves the removal of a small amount of placental tissue between the tenth and twelfth week of pregnancy. Under ultrasound guidance via the vagina, a narrow tube is inserted into the placenta and a small biopsy is taken.

Alternatively, the placental biopsy may be obtained from subjects with natural abortion of the pregnancy in order to establish the cause of said premature abortion. This information is an important predictive tool in view of future pregnancies.

The RNA level may be detected using standard quantitative RNA measurement tools known in the art. Non-limiting examples include hybridization-based analysis, microarray expression analysis, digital gene expression (DGE), RNA-in-situ hybridization (RISH), Northern-blot analysis and the like; PCR, RT-PCR, RT-qPCR, end-point PCR, digital PCR or the like; supported oligonucleotide detection, pyrosequencing, polony cyclic sequencing by synthesis, simultaneous bi-directional sequencing, single-molecule sequencing, single molecule real time sequencing, true single molecule sequencing, hybridization-assisted nanopore sequencing and sequencing by synthesis.

The various aspects and embodiments taught herein may further rely on comparing the quantity of biomarkers measured in samples and the measurement or score of parameters in patients with reference values, wherein said reference values represent known predictions, diagnoses and/or prognoses of diseases or conditions as taught herein.

For example, distinct reference values may represent the prediction of a risk (e.g., an abnormally elevated risk) of having a given disease or condition as taught herein vs. the prediction of no or normal risk of having said disease or condition. In another example, distinct reference values may represent predictions of differing degrees of risk of having such disease or condition.

In a further example, distinct reference values may represent the diagnosis of a given disease or condition as taught herein vs. the diagnosis of no such disease or condition (such as, e.g., the diagnosis of healthy, or recovered from said disease or condition, etc.). In another example, distinct reference values may represent the diagnosis of such disease or condition of varying severity.

In yet another example, distinct reference values may represent a good prognosis for a given disease or condition as taught herein vs. a poor prognosis for said disease or condition. In a further example, distinct reference values may represent varyingly favourable or unfavourable prognoses for such disease or condition.

Such comparison may generally include any means to determine the presence or absence of at least one difference and optionally of the size of such difference between values being compared. A comparison may include a visual inspection, an arithmetical or statistical comparison of measurements. Such statistical comparisons include, but are not limited to, applying a rule.

Reference values may be established according to known procedures previously employed for other biomarkers and parameters. For example, a reference value may be established in an individual or a population of individuals characterised by a particular diagnosis, prediction and/or prognosis of said disease or condition (i.e., for whom said diagnosis, prediction and/or prognosis of the disease or condition holds true). Such population may comprise without limitation ≧2, ≧10, ≧100, or even several hundreds or more individuals.

In an embodiment, reference value(s) as intended herein may convey absolute quantities of the biomarkers, peptides, polypeptides, proteins or a fragment thereof as intended herein. In another embodiment, the quantity of the biomarkers, peptides, polypeptides, proteins or a fragment thereof in a sample from a tested subject may be determined directly relative to the reference value (e.g., in terms of increase or decrease, or fold-increase or fold-decrease). Advantageously, this may allow the comparison of the quantity of the biomarkers, peptides, polypeptides, proteins or a fragment thereof in the sample from the subject with the reference value (in other words to measure the relative quantity of the biomarkers, peptides, polypeptides, proteins or a fragment thereof in the sample from the subject vis-à-vis the reference value) without the need first to determine the respective absolute quantities of the biomarkers, peptides, polypeptides, proteins or a fragment thereof.

The expression level or presence of a biomarker in a sample of a patient may sometimes fluctuate, i.e. increase or decrease significantly without change (appearance of, worsening or improving) of symptoms. In such an event, the marker change precedes the change in symptoms and becomes a more sensitive measure than symptom change. Therapeutic intervention may be initiated earlier and be more effective than waiting for deteriorating symptoms.

Also disclosed is a method or algorithm for determining a significant change in the level of any one or more of the markers as taught herein or a fragment thereof in a certain patient, which is indicative for change (worsening or improving) in clinical status. In addition, the invention allows establishing the diagnosis that the subject is recovering or has recovered from a given disease or condition as taught herein.

In an embodiment the present methods may include a step of establishing such reference value(s). In an embodiment, the present kits and devices may include means for establishing a reference value of the quantity of any one or more of the markers as taught herein or a fragment thereof for a particular prediction, diagnosis and/or prognosis of a given disease or condition as taught herein. Such means may for example comprise one or more samples (e.g., separate or pooled samples) from one or more individuals characterised by said particular prediction, diagnosis and/or prognosis of said disease or condition.

The various aspects and embodiments taught herein may further entail finding a deviation or no deviation between the quantity of any one or more markers as taught herein or a fragment thereof measured in a sample from a subject and a given reference value.

A “deviation” of a first value from a second value may generally encompass any direction (e.g., increase: first value>second value; or decrease: first value<second value) and any extent of alteration.

For example, a deviation may encompass a decrease in a first value by, without limitation, at least about 10% (about 0.9-fold or less), or by at least about 20% (about 0.8-fold or less), or by at least about 30% (about 0.7-fold or less), or by at least about 40% (about 0.6-fold or less), or by at least about 50% (about 0.5-fold or less), or by at least about 60% (about 0.4-fold or less), or by at least about 70% (about 0.3-fold or less), or by at least about 80% (about 0.2-fold or less), or by at least about 90% (about 0.1-fold or less), relative to a second value with which a comparison is being made.

For example, a deviation may encompass an increase of a first value by, without limitation, at least about 10% (about 1.1-fold or more), or by at least about 20% (about 1.2-fold or more), or by at least about 30% (about 1.3-fold or more), or by at least about 40% (about 1.4-fold or more), or by at least about 50% (about 1.5-fold or more), or by at least about 60% (about 1.6-fold or more), or by at least about 70% (about 1.7-fold or more), or by at least about 80% (about 1.8-fold or more), or by at least about 90% (about 1.9-fold or more), or by at least about 100% (about 2-fold or more), or by at least about 150% (about 2.5-fold or more), or by at least about 200% (about 3-fold or more), or by at least about 500% (about 6-fold or more), or by at least about 700% (about 8-fold or more), or like, relative to a second value with which a comparison is being made.

Preferably, a deviation may refer to a statistically significant observed alteration. For example, a deviation may refer to an observed alteration which falls outside of error margins of reference values in a given population (as expressed, for example, by standard deviation or standard error, or by a predetermined multiple thereof, e.g., ±1×SD or ±2×SD, or ±1×SE or ±2×SE). Deviation may also refer to a value falling outside of a reference range defined by values in a given population (for example, outside of a range which comprises 40%, 50%, 60%, 70%, 75% or 80% or 85% or 90% or 95% or even 100% of values in said population).

In a further embodiment, a deviation may be concluded if an observed alteration is beyond a given threshold or cut-off. Such threshold or cut-off may be selected as generally known in the art to provide for a chosen sensitivity and/or specificity of the diagnosis, prediction and/or prognosis methods, e.g., sensitivity and/or specificity of at least 50%, or at least 60%, or at least 70%, or at least 80%, or at least 85%, or at least 90%, or at least 95%.

The present invention further provides kits or devices as set forth above for the diagnosis, prediction, prognosis and/or monitoring of any one disease or condition as taught herein comprising means for detecting the level of biomarker(s) for instance comprised in test panels as taught herein in a sample of the patient. In a preferred embodiment, such a kit or kits may be used in clinical settings or at home. The kit may be used for diagnosing said disease or condition, for monitoring the effectiveness of treatment of a subject suffering from said disease or condition with an agent, or for preventive screening of subjects for the occurrence of said disease or condition in said subject.

In a clinical setting, the kit or device may be in the form of a bed-side device or in an emergency team setting, e.g. as part of the equipment of an ambulance or other moving emergency vehicle or team equipment or as part of a first-aid kit. The diagnostic kit or device may assist a medical practitioner, a first aid helper, or nurse to decide whether the patient under observation is developing a disease or condition as taught herein, after which appropriate action or treatment can be performed.

A home-test kit gives the patient a readout which he/she may communicate to a medicinal practitioner, a first aid helper or to the emergency department of a hospital, after which appropriate action can be taken. Such a home-test device is of particular interest for people having either a history of, or are at risk of suffering from any one disease or condition as taught herein.

Non-limiting examples are: systems comprising specific binding molecules for the requisite biomarker(s) attached to a solid phase, e.g. lateral flow strips or dipstick devices and the like well known in the art. One non-limiting example to perform a biochemical assay is to use a test-strip and labelled antibodies which combination does not require any washing of the membrane. The test strip is well known, for example, in the field of pregnancy testing kits where an anti-hCG antibody is present on the support, and is carried complexed with hCG by the flow of urine onto an immobilised second antibody that permits visualisation. Other non-limiting examples of such home test devices, systems or kits can be found for example in the following U.S. Pat. Nos. 6,107,045, 6,974,706, 5,108,889, 6,027,944, 6,482,156, 6,511,814, 5,824,268, 5,726,010, 6,001,658 or U.S. patent applications: 2008/0090305 or 2003/0109067.

In a preferred embodiment, the invention provides a lateral flow device or dipstick. Such dipstick comprises a test strip allowing migration of a sample by capillary flow from one end of the strip where the sample is applied to the other end of such strip where presence of an analyte in said sample is measured. In another embodiment, the invention provides a device comprising a reagent strip. Such reagent strip comprises one or more test pads which when wetted with the sample, provide a colour change in the presence of an analyte and/or indicate the concentration of the protein in said sample.

In order to obtain a semi-quantitative test strip in which only a signal is formed once the level of the requisite biomarker(s) in the sample is higher than a certain predetermined threshold level or value, a predetermined amount of fixed capture antibodies for the biomarker(s) may be present on the test strip. This enables the capture of a certain amount of the biomarker(s) present in the sample, corresponding to the threshold level or value as predetermined. The remaining amount of biomarker(s) (if any) bound by e.g. a conjugated or labelled binding molecules may then be allowed to migrate to a detection zone which subsequently only produces a signal if the level of the biomarker(s) in the sample is higher than the predetermined threshold level or value.

Another possibility to determine whether the amount of any the requisite biomarker(s) in the sample is below or above a certain threshold level or value, is to use a primary capturing antibody capturing all said biomarker(s) present in the sample, in combination with a labelled secondary antibody, developing a certain signal or colour when bound to the solid phase. The intensity of the colour or signal may then either be compared to a reference colour or signal chart indicating that when the intensity of the signal is above a certain threshold signal, the test is positive. Alternatively, the amount or intensity of the colour or signal may be measured with an electronic device comprising e.g. a light absorbance sensor or light emission meter, resulting in a numerical value of signal intensity or colour absorbance formed, which may then be displayed to the subject in the form of a negative result if said numerical value is below the threshold value or a positive result if said numerical value is above the threshold value. This embodiment is of particular relevance in monitoring the level of said biomarker(s) in a patient over a period of time.

The reference value or range can e.g. be determined using the home device in a period wherein the subject is free of a given disease or condition, giving the patient an indication of her base-line level of the biomarker(s). Regularly using the home test device will thus enable the subject to notice a sudden change in levels of said biomarker(s) as compared to the base-line level, which enable him/her to contact a medical practitioner.

Alternatively, the reference value may be determined in the subject suffering from a given disease or condition as taught herein, which then indicates her personal “risk level” for the biomarker(s), i.e. the level of the biomarker(s) which indicates he/she is or will soon be exposed to said disease or condition. This risk level is interesting for monitoring the disease progression or for evaluating the effect of the treatment.

Furthermore, the reference value or level may be established through combined measurement results in subjects with highly similar disease states or phenotypes (e.g. all having no disease or condition as taught herein or having said disease or condition).

Non-limiting examples of semi-quantitative tests known in the art, the principle of which may be used for the home test device according to the present invention are the HIV/AIDS test or Prostate Cancer tests sold by Sanitoets. The home prostate test is a rapid test intended as an initial semi-quantitative test to detect PSA blood levels higher than 4 ng/ml in whole blood. The typical home self-test kit comprises the following components: a test device to which the blood sample is to be administered and which results in a signal when the protein level is above a certain threshold level, an amount of diluent e.g. in dropper pipette to help the transfer of the analytes (i.e. the protein of interest) from the sample application zone to the signal detection zone, optionally an empty pipette for blood specimen collection, a finger pricking device, optionally a sterile swab to clean the area of pricking and instructions of use of the kit.

Similar tests are also known for e.g. breast cancer detection and CRP-protein level detection in view of cardiac risk home tests. The latter test encompasses the sending of the test result to a laboratory, where the result is interpreted by a technical or medical expert. Such telephone or internet based diagnosis of the patient's condition is of course possible and advisable with most of the kits, since interpretation of the test result is often more important than conducting the test. When using an electronic device as mentioned above which gives a numerical value of the level of protein present in the sample, this value may of course easily be communicated through telephone, mobile telephone, satellite phone, E-mail, internet or other communication means, warning a hospital, a medicinal practitioner or a first aid team that a person is, or may be at risk of, suffering from the disease or condition as taught herein. A non-limiting example of such a system is disclosed in U.S. Pat. No. 6,482,156.

The presence and/or concentration of biomarker(s) in a sample may be measured by surface plasmon resonance (SPR) using a chip having binding molecule for said biomarker(s) immobilized thereon, fluorescence resonance energy transfer (FRET), bioluminescence resonance energy transfer (BRET), fluorescence quenching, fluorescence polarization measurement or other means known in the art. Any of the binding assays described may be used to determine the presence and/or concentration of any biomarker(s) in a sample. To do so, binding molecules for the biomarker(s) are reacted with a sample, and the concentration of the biomarker(s) is measured as appropriate for the binding assay being used. To validate and calibrate an assay, control reactions using different concentrations of standard biomarker(s) and/or binding molecule therefore may be performed. Where solid phase assays are employed, after incubation, a washing step is performed to remove unbound markers. Bound biomarker is measured as appropriate for the given label (e.g., scintillation counting, fluorescence, antibody-dye etc.). If a qualitative result is desired, controls and different concentrations may not be necessary. Of course, the roles of said biomarker(s) and binding molecule may be switched; the skilled person may adapt the method so binding molecule is applied to sample, at various concentrations of sample.

A “binding molecule for any one or more markers as taught herein or a fragment thereof” is any substance that binds specifically to any one or more markers as taught herein or a fragment thereof. Examples of a binding molecule for any one or more markers as taught herein or a fragment thereof, includes, but is not limited to an antibody, a polypeptide, a peptide, a lipid, a carbohydrate, a nucleic acid, peptide-nucleic acid, small molecule, small organic molecule, or other drug candidate. A binding molecule for any one or more markers as taught herein or a fragment thereof may be natural or synthetic compound, including, for example, synthetic small molecule, compound contained in extracts of animal, plant, bacterial or fungal cells, as well as conditioned medium from such cells. Alternatively, binding molecule for any one or more markers as taught herein or a fragment thereof may be an engineered protein having binding sites for any one or more markers as taught herein or a fragment thereof. According to an aspect of the invention, a binding molecule for any one or more markers as taught herein or a fragment thereof binds specifically to any one or more markers as taught herein or a fragment thereof with an affinity better than 10⁻⁶ M. A suitable binding molecule for any one or more markers as taught herein or a fragment thereof may be determined from its binding with a standard sample of any one or more markers as taught herein or a fragment thereof. Methods for determining the binding between binding molecules for any one or more markers as taught herein or a fragment thereof and any one or more markers as taught herein or a fragment thereof are known in the art. As used herein, the term antibody includes, but is not limited to, polyclonal antibodies, monoclonal antibodies, humanised or chimeric antibodies, engineered antibodies, and biologically functional antibody fragments (e.g. scFv, nanobodies, Fv, etc) sufficient for binding of the antibody fragment to the protein. Such antibody may be commercially available antibody against any one or more markers as taught herein or a fragment thereof, such as, for example, a mouse, rat, human or humanised monoclonal antibody.

In a preferred embodiment, the binding molecule or agent is capable of binding both the mature membrane- or cell-bound protein or fragment of any one or more markers as taught herein or a fragment thereof. In a more preferred embodiment, the binding agent or molecule is specifically binding or detecting the soluble form, preferably the plasma circulating form of any one or more markers as taught herein or a fragment thereof.

According to one aspect of the invention, the binding molecule for any one or more markers as taught herein or a fragment thereof is labelled with a tag that permits detection with another agent (e.g. with a probe binding partner). Such tags can be, for example, biotin, streptavidin, his-tag, myc tag, maltose, maltose binding protein or any other kind of tag known in the art that has a binding partner. Example of associations which can be utilised in the probe:binding partner arrangement may be any, and includes, for example biotin:streptavidin, his-tag:metal ion (e.g. Ni²⁺), maltose:maltose binding protein.

The specific-binding agents, peptides, polypeptides, proteins, biomarkers etc. in the present kits may be in various forms, e.g., lyophilised, free in solution or immobilised on a solid phase. They may be, e.g., provided in a multi-well plate or as an array or microarray, or they may be packaged separately and/or individually. The may be suitably labelled as taught herein. Said kits may be particularly suitable for performing the assay methods of the invention, such as, e.g., immunoassays, ELISA assays, mass spectrometry assays, and the like.

The term “modulate” generally denotes a qualitative or quantitative alteration, change or variation specifically encompassing both increase (e.g., activation) or decrease (e.g., inhibition), of that which is being modulated. The term encompasses any extent of such modulation.

For example, where modulation effects a determinable or measurable variable, then modulation may encompass an increase in the value of said variable by at least about 10%, e.g., by at least about 20%, preferably by at least about 30%, e.g., by at least about 40%, more preferably by at least about 50%, e.g., by at least about 75%, even more preferably by at least about 100%, e.g., by at least about 150%, 200%, 250%, 300%, 400% or by at least about 500%, compared to a reference situation without said modulation; or modulation may encompass a decrease or reduction in the value of said variable by at least about 10%, e.g., by at least about 20%, by at least about 30%, e.g., by at least about 40%, by at least about 50%, e.g., by at least about 60%, by at least about 70%, e.g., by at least about 80%, by at least about 90%, e.g., by at least about 95%, such as by at least about 96%, 97%, 98%, 99% or even by 100%, compared to a reference situation without said modulation.

Preferably, modulation of the activity and/or level of intended target(s) (e.g., any one or more markers, nucleic acids, peptides, polypeptides or proteins as taught herein) may be specific or selective, i.e., the activity and/or level of intended target(s) may be modulated without substantially altering the activity and/or level of random, unrelated (unintended, undesired) targets.

Reference to the “activity” of a target may generally encompass any one or more aspects of the biological activity of the target, such as without limitation any one or more aspects of its biochemical activity, enzymatic activity, signalling activity and/or structural activity, e.g., within a cell, tissue, organ or an organism.

In the context of therapeutic or prophylactic targeting of a target, the reference to the “level” of a target may preferably encompass the quantity and/or the availability (e.g., availability for performing its biological activity) of the target, e.g., within a cell, tissue, organ or an organism.

For example, the level of a target may be modulated by modulating the target's expression and/or modulating the expressed target. Modulation of the target's expression may be achieved or observed, e.g., at the level of heterogeneous nuclear RNA (hnRNA), precursor mRNA (pre-mRNA), mRNA or cDNA encoding the target. By means of example and not limitation, decreasing the expression of a target may be achieved by methods known in the art, such as, e.g., by transfecting (e.g., by electroporation, lipofection, etc.) or transducing (e.g., using a viral vector) a cell, tissue, organ or organism with an antisense agent, such as, e.g., antisense DNA or RNA oligonucleotide, a construct encoding the antisense agent, or an RNA interference agent, such as siRNA or shRNA, or a ribozyme or vectors encoding such, etc. By means of example and not limitation, increasing the expression of a target may be achieved by methods known in the art, such as, e.g., by transfecting (e.g., by electroporation, lipofection, etc.) or transducing (e.g., using a viral vector) a cell, tissue, organ or organism with a recombinant nucleic acid which encodes said target under the control of regulatory sequences effecting suitable expression level in said cell, tissue, organ or organism. By means of example and not limitation, the level of the target may be modulated via alteration of the formation of the target (such as, e.g., folding, or interactions leading to formation of a complex), and/or the stability (e.g., the propensity of complex constituents to associate to a complex or disassociate from a complex), degradation or cellular localisation, etc. of the target.

The term “antisense” generally refers to a molecule designed to interfere with gene expression and capable of specifically binding to an intended target nucleic acid sequence. Antisense agents typically encompass an oligonucleotide or oligonucleotide analogue capable of specifically hybridising to the target sequence, and may typically comprise, consist essentially of or consist of a nucleic acid sequence that is complementary or substantially complementary to a sequence within genomic DNA, hnRNA, mRNA or cDNA, preferably mRNA or cDNA corresponding to the target nucleic acid. Antisense agents suitable herein may typically be capable of hybridising to their respective target at high stringency conditions, and may hybridise specifically to the target under physiological conditions.

The term “ribozyme” generally refers to a nucleic acid molecule, preferably an oligonucleotide or oligonucleotide analogue, capable of catalytically cleaving a polynucleotide. Preferably, a “ribozyme” may be capable of cleaving mRNA of a given target protein, thereby reducing translation thereof. Exemplary ribozymes contemplated herein include, without limitation, hammer head type ribozymes, ribozymes of the hairpin type, delta type ribozymes, etc. For teaching on ribozymes and design thereof, see, e.g., U.S. Pat. No. 5,354,855, U.S. Pat. No. 5,591,610, Pierce et al. 1998 (Nucleic Acids Res 26: 5093-5101), Lieber et al. 1995 (Mol Cell Biol 15: 540-551), and Benseler et al. 1993 (J Am Chem Soc 115: 8483-8484).

“RNA interference” or “RNAi” technology is routine in the art and suitable RNAi agents intended herein may include inter alia short interfering nucleic acids (siNA), short interfering RNA (sRNA), double-stranded RNA (dsRNA), micro-RNA (miRNA), and short hairpin RNA (shRNA) molecules as known in the art. For teaching on RNAi molecules and design thereof, see inter alia Elbashir et al. 2001 (Nature 411: 494-501), Reynolds et al. 2004 (Nat Biotechnol 22: 326-30), http://maidesigner.invitrogen.com/maiexpress, Wang & Mu 2004 (Bioinformatics 20: 1818-20), Yuan et al. 2004 (Nucleic Acids Res 32 (Web Server issue): W130-4), by M Sohail 2004 (“Gene Silencing by RNA Interference: Technology and Application”, 1^{st} ed., CRC, ISBN 0849321417), U Schepers 2005 (“RNA Interference in Practice: Principles, Basics, and Methods for Gene Silencing in C. elegans, Drosophila, and Mammals”, 1^{st} ed., Wiley-VCH, ISBN 3527310207), and D R Engelke & J J Rossi 2005 (“Methods in Enzymology, Volume 392: RNA Interference”, 1^{st} ed., Academic Press, ISBN 0121827976).

The term “pharmaceutically acceptable” as used herein is consistent with the art and means compatible with the other ingredients of a pharmaceutical composition and not deleterious to the recipient thereof.

As used herein, “carrier” or “excipient” includes any and all solvents, diluents, buffers (such as, e.g., neutral buffered saline or phosphate buffered saline), solubilisers, colloids, dispersion media, vehicles, fillers, chelating agents (such as, e.g., EDTA or glutathione), amino acids (such as, e.g., glycine), proteins, disintegrants, binders, lubricants, wetting agents, emulsifiers, sweeteners, colourants, flavourings, aromatisers, thickeners, agents for achieving a depot effect, coatings, antifungal agents, preservatives, antioxidants, tonicity controlling agents, absorption delaying agents, and the like. The use of such media and agents for pharmaceutical active substances is well known in the art. Except insofar as any conventional media or agent is incompatible with the active substance, its use in the therapeutic compositions may be contemplated.

The present active substances (agents) may be used alone or in combination with any therapies known in the art for the disease and conditions as taught herein (“combination therapy”). Combination therapies as contemplated herein may comprise the administration of at least one active substance of the present invention and at least one other pharmaceutically or biologically active ingredient. Said present active substance(s) and said pharmaceutically or biologically active ingredient(s) may be administered in either the same or different pharmaceutical formulation(s), simultaneously or sequentially in any order.

The dosage or amount of the present active substances (agents) used, optionally in combination with one or more other active compound to be administered, depends on the individual case and is, as is customary, to be adapted to the individual circumstances to achieve an optimum effect. Thus, it depends on the nature and the severity of the disorder to be treated, and also on the sex, age, body weight, general health, diet, mode and time of administration, and individual responsiveness of the human or animal to be treated, on the route of administration, efficacy, metabolic stability and duration of action of the compounds used, on whether the therapy is acute or chronic or prophylactic, or on whether other active compounds are administered in addition to the agent(s) of the invention.

Without limitation, depending on the type and severity of the disease, a typical daily dosage might range from about 1 μg/kg to 100 mg/kg of body weight or more, depending on the factors mentioned above. For repeated administrations over several days or longer, depending on the condition, the treatment is sustained until a desired suppression of disease symptoms occurs. A preferred dosage of the active substance of the invention may be in the range from about 0.05 mg/kg to about 10 mg/kg of body weight. Thus, one or more doses of about 0.5 mg/kg, 2.0 mg/kg, 4.0 mg/kg or 10 mg/kg (or any combination thereof) may be administered to the patient. Such doses may be administered intermittently, e.g., every week or every two or three weeks.

As used herein, a phrase such as “a subject in need of treatment” includes subjects that would benefit from treatment of a given disease or condition as taught herein. Such subjects may include, without limitation, those that have been diagnosed with said condition, those prone to contract or develop said condition and/or those in whom said condition is to be prevented.

The terms “treat” or “treatment” encompass both the therapeutic treatment of an already developed disease or condition, as well as prophylactic or preventative measures, wherein the aim is to prevent or lessen the chances of incidence of an undesired affliction, such as to prevent the chances of contraction and progression of a disease or condition as taught herein. Beneficial or desired clinical results may include, without limitation, alleviation of one or more symptoms or one or more biological markers, diminishment of extent of disease, stabilised (i.e., not worsening) state of disease, delay or slowing of disease progression, amelioration or palliation of the disease state, and the like. “Treatment” may also mean prolonging survival as compared to expected survival if not receiving treatment.

The term “prophylactically effective amount” refers to an amount of an active compound or pharmaceutical agent that inhibits or delays in a subject the onset of a disorder as being sought by a researcher, veterinarian, medical doctor or other clinician. The term “therapeutically effective amount” as used herein, refers to an amount of active compound or pharmaceutical agent that elicits the biological or medicinal response in a subject that is being sought by a researcher, veterinarian, medical doctor or other clinician, which may include inter alia alleviation of the symptoms of the disease or condition being treated. Methods are known in the art for determining therapeutically and prophylactically effective doses for the present compounds.

The above aspects and embodiments are further supported by the following non-limiting examples.

### EXAMPLES

### Example 1

### MASSterclass® Targeted Protein Quantitation

### MASSTERCLASS® Experimental Setup

MASSterclass® assays use targeted tandem mass spectrometry with stable isotope dilution as an end-stage peptide quantitation system (also called Multiple Reaction Monitoring (MRM) and Single Reaction Monitoring (SRM). The targeted peptide is specific (i.e., proteotypic) for the specific protein of interest. i.e., the amount of peptide measured is directly related to the amount of protein in the original sample. To reach the specificity and sensitivity needed for biomarker quantitation in complex samples, peptide fractionation precedes the end-stage quantitation step.

A suitable MASSTERCLASS® assay may include the following steps:
Plasma/serum sample
Depletion of human albumin and IgG (complexity reduction on protein level) using affinity capture with anti-albumin and anti-IgG antibodies using ProteoPrep spin columns (Sigma Aldrich)
Spiking of known amounts of isotopically labelled peptides. These peptides has the same amino acid sequence as the proteotypic peptides of interest, typically with one isotopically labelled amino acid built in to generate a mass difference. During the entire process, the labelled peptide has identical chemical and chromatographic behaviour as the endogenous peptide, except during the end-stage quantitation step which is based on molecular mass.
Tryptic digest. The proteins in the depleted serum/plasma sample are digested into peptides using trypsin. This enzyme cleaves proteins C-terminally from lysine and argninine, except when a proline is present C-terminally of the lysine or arginine. Before digestion, proteins are denatured by boiling, which renders the protein molecule more accessible for the trypsin activity during the 16 h incubation at 37° C.
Peptide-based fractionation: The charged peptide molecules are separated based on their specific isoelectric property. As there is no pl difference between the endogenous peptide and the isotopically labelled variant, they co-elute. Only those fractions containing the monitored peptides, or pools thereof, are selected and proceed to the next level of fractionation.
LC-MS/MS based quantitation, including further separation on reversed phase (C18) nanoLC (PepMap C18; Dionex) and MS/MS: tandem mass spectrometry using MRM (4000 QTRAP; ABI) or SRM (Vantage TSQ; Thermo Scientific) mode. The LC column is connected to an electrospray needle connected to the source head of the mass spectrometer. As material elutes from the column, molecules are ionized and enter the mass spectrometer in the gas phase. The peptide that is monitored is specifically selected to pass the first quadrupole (Q1), based on its mass to charge ratio (m/z). The selected peptide is then fragmented in a second quadrupole (Q2) which is used as a collision cell. The resulting fragments then enter the third quadrupole (Q3). Depending on the instrument settings (determined during the assay development phase) only a specific peptide fragment or specific peptide fragments (or so called transitions) are selected for detection.
The combination of the m/z of the monitored peptide and the m/z of the monitored fragment of this peptide is called a transition. This process can be performed for multiple transitions during one experiment. Both the endogenous peptide (analyte) and its corresponding isotopically labelled synthetic peptide (internal standard) elute at the same retention time, and are measured in the same LC-MS/MS experiment.
The MASSterclass® readout is defined by the ratio between the area under the peak specific for the analyte and the area under the peak specific for the synthetic isotopically labelled analogue (internal standard). MASSterclass® readouts are directly related to the original concentration of the protein in the sample. MASSterclass® readouts can therefore be compared between different samples and groups of samples.

A typical MASSTERCLASS® protocol followed in the present study:
25 μL of plasma is subjected to a depletion of human albumin and IgG (ProteoPrep spin columns; Sigma Aldrich) according to the manufacturer's protocol, except that 20 mM NH₄HCO₃ was used as the binding/equilibration buffer.
The depleted sample (225 μL) is denatured for 15 min at 95° C. and immediately cooled on ice
2 pmol of each isotopically labeled peptide (custom made ‘Heavy AQUA’ peptide; Thermo Scientific) is spiked in the sample
20 μg trypsin is added to the sample and digestion is allowed for 16 h at 37° C.
Half of the resulting sample is applied to pl-based separation. Fractions containing the peptides of interest are pooled together, dried and resuspended in 0.1% formic acid.
20 μL of the final solution is separated using reverse-phase NanoLC with on-line MS/MS in SRM mode:
   Column: PepMap C18, 75 μm I.D.×25 cm L, 100 Å pore diameter, 5 μm particle size
   Solvent A: 0.1% formic acid
   Solvent B: 80% acetonitrile, 0.1% formic acid
   Gradient: 30 min; 2%-55% Solvent B
   MS/MS in SRM mode: method contains the transitions for the analyte as well as for the synthetic, labeled peptide.
The used transitions were experimentally determined and selected during protein assay development

The following table summarizes the peptides used for the different MASSterclass® assays

| Gene name | SwissProt entry | Peptide code | Peptide sequence | Seq ID |
|---|---|---|---|---|
| MRC1 | MRC1_HUMAN | MC102 | SQGPEIVEVEK | 4 |
| PTX3 | PTX3_HUMAN | MC246 | ALAAVLEELR | 6 |
| EXT2 | EXT2_HUMAN | MC451 | EDLEALQVK | 11 |
| EXT2 | EXT2_HUMAN | MC452 | LPADSPIPER | 12 |
| IL1R2 | IL1R2_HUMAN | MC477 | EETIPVIISPLK | 8 |
| IL1R2 | IL1R2_HUMAN | MC479 | LEGEPVALR | 9 |
| PRTN3 | PRTN3_HUMAN | MC508 | LFPDFFTR | 2 |
| GSS | GSHB_HUMAN | MC382 | AIENELLAR | 14 |
| GSS | GSHB_HUMAN | MC383 | TFEDISEK | 15 |
| VCAM1 | VCAM1_HUMAN | MC029 | SLEVTFTPVIEDIGK | 17 |
| PSMA3 | PSA3_HUMAN | MC510 | DGVVFGVEK | 19 |
| NID1 | NID1_HUMAN | MC044 | VLFETDLVNPR | 21 |
| GOLM1 | GOLM1_HUMAN | MC067 | DTINLLDQR | 23 |
| ATF6A | ATF6A_HUMAN | MC349 | EAQDTSDGIIQK | 25 |
| ICAM1 | ICAM1_HUMAN | MC023 | EPAVGEPAEVTTTVLVR | 27 |
| PIGR | PIGR_HUMAN | MC333 | IIEGEPNLK | 29 |
| CALU | CALU_HUMAN | MC429 | WIYEDVER | 31 |
| GPLD1 | PHLD_HUMAN | MC018 | IADVTSGLIGGEDGR | 33 |
| FGL1 | FGL1_HUMAN | MC379 | DYENGFGNFVQK | 35 |

For the majority of markers multiple assays are available: these are either based on different spiked peptides, same peptides but different transition measured, or different branch (with or without prefractionation). Each assay has a code with the following format:

### PRCxx_MCxxx_TRxxBr1_Ma1

PRCxx: protein code
MCxxx: peptide code
TRxx: transition code
Brx: br1 for branch1 measurements (without prefractionation); br4 for branch 4 measurements (with PI based prefractionation)
Ma1: mass spectrometer used: for this screening MASSterclass® units were used

### Example 2

### Description of Cohort Used to Identify Markers

Between 2004 and 2005, all patients with signs of systemic inflammatory response syndrome (SIRS) and suspicion of sepsis within the Utrecht Medical Center (Prof Verhoef, Utrecht, the Netherlands) were included in this study. A sample was taken for blood culture and at the same time a blood sample was collected for future biochemical analysis. In total over 1000 patients were included coming from different hospital departments. Final adjudicated diagnosis and classification as either SIRS or SEPSIS was done by three independent physicians based on all available clinical data (patient charts, culture of micro-organisms, biochemical markers, treatment and response to treatment, outcome). If left uncertain, the patient was called “possible sepsis”. SIRS, sepsis and severe sepsis definitions used were as set out in the sepsis guidelines (Levy et al., 2003, supra), CDC criteria or as defined herein. Sepsis was defined as proven infection based on cultures (blood or other) or based on clinical presentation of the patient. Severe sepsis was defined as sepsis plus organ dysfunction. For each sepsis patients the focus of primary infection was recorded and these were sub-grouped in respiratory tract, urogenital tract, gastro-intestinal tract or other. If other cultures than blood cultures were taken, this was recorded as well as the isolated micro-organism from the cultures. If antibiotics therapy was given, this was recorded, as well as whether the therapy turned out to be appropriate. For each patient the overall SOFA (Sequential Organ Failure Assessment) score was calculated based on the separate scores for respiratory, cardiovascular, hepatic, coagulation, renal and neurological systems. Finally the outcome (survivor versus non-survivor) at 28 days post day of blood sampling was recorded.

A subset of this database was used in this analysis. The set was sub-selected for community acquired infections (blood culture within 48 hrs of hospital admission) and patients with septic shock or under immune suppression and with uncertain final diagnosis were excluded. Table 2 summarizes the most important patient characteristics.

TABLE 2
Summary of patient characteristics

| | SIRS (n = 51) | Sepsis (n = 119) |
|---|---|---|
| Age | 57 (43-70) | 60 (43.5-70) |
| Gender (% male) | 55 | 55 |
| SOFA score | 1 (0.5-3) | 2 (1-3) |
| % non-survivor | 18 (n = 9) | 11 (n = 13) |
| % bacteraemia | 0 | 30 (n = 36) |
| Focus of infection: | | |
| Respiratory tract | 0 | 25% (n = 29) |
| Urinary tract | 0 | 25% (n = 30) |
| Gastro-intestinal tract | 0 | 25% (n = 30) |
| other | 0 | 25% (n = 30) |
| White blood cell | 10.6 (6.3-15) | 15.2 (10.9-17.2) |
| count (×10⁹ cells/L) | | |
| Body temperature (C.) | 38.6 (38.2-39.2) | 38.7 (38.2-39.2) |
| Heart rate (beats/min) | 104 (96-113) | 101 (92-108) |
| CRP (ug/mL) | 70 (31.5-138) | 88 (49-171) |
| PCT (ug/mL) | 0.2 (0.12-0.41) | 0.6 (0.16-3) |

### Example 3

### Univariate and Multivariate Analysis to Identify Novel Markers

The overall goal of the study was to identify new candidate biomarkers for early and accurate diagnosis of systemic inflammatory conditions, and in particular sepsis. To that end several relevant outcomes were defined and markers were scored for their diagnostic performance to identify these outcomes. For this analysis both single marker performance was assessed as well as marker combinations, with a maximum of 3 markers per panel in order not to overfit the data. In an independent analysis marker dependencies were investigated, i.e. associations of marker levels with clinical variables and levels of other markers.

### Definition of Outcomes

Based on the available clinical data and the goal of the study, three types of diagnostic questions were looked at:
Infection markers: discriminate between SIRS and SEPSIS patients, where sepsis is defined based on different definitions
   All Sepsis: all sepsis patients in which the sepsis classification is based on the adjudicated diagnosis of the independent physicians
   Mild sepsis (coded here mild infection): all patients with a proven infection but who did not show signs of organ failure at time of blood sampling (i.e., exclusion of severe sepsis patients)
   Bacteraemia: defined as patients with a positive blood culture.
Organ failure markers: discriminate between patients where at time of sampling no organ failure is diagnosed and patients were at least one failing organ is diagnosed.
Prognostic markers: markers that can predict patient mortality 28 days post diagnosis and blood sampling.

Further analysis looking into more specific subpopulations such as focus of infection, type of organ failure etc was not performed in a holistic way, but on a marker candidate basis. For these analyses the MedCalc Software was used.

An overview of the different patient subgroups and the defined outcomes used in this analysis can be found in Table 3.

TABLE 3
Overview of the defined outcomes and patient
subgroups used for data analysis

| Outcome | Controls (#) | Cases (#) |
|---|---|---|
| Infection | SIRS (n = 51) | All sepsis (n = 119); Sepsis + |
| | | severe sepsis |
| Mild infection | SIRS (n = 51) | sepsis (n = 59) |
| Organ failure | No organ failure (n = 84) | Organ failure (n = 86) |
| Mortality | Survivors (n = 148) | Non survivors (n = 22) |
| Bacteraemia | No bacteraemia detected | Bacteria in blood stream |
| | (n = 134) | (n = 36) |

### Univariate Analysis

For all markers per MASSterclass® assay the discriminatory power for the different outcomes was calculated. Median levels, interquartile ranges and fold differences were reported as appropriate. ROC (receiver operating characteristic) curves were constructed. The estimated and 95% confidence intervals for area under the curve (AUC) were computed using the Delong method. The AUC was compared between two markers using a non-parametric approach.

### Multivariate Analysis

The multivariate analysis was aimed at finding marker combinations that either improve on its single components or that improve upon performance of procalcitonin (PCT). To this end logistic regression models were computed for combinations of a number of preselected markers. The selection of these single markers or covariates was done based on (1) their clinical relevance, (2) their discriminative performance as a single marker (see univariate analysis), (3) on the number of missing values.

The analyses were conducted using the log-transformed analyte concentrations, either relative concentrations (MASSterclass® measurements) or absolute levels (immune-assay based measurements for PCT). For each logistic regression model, receiver operator characteristic (ROC) analysis was performed to calculate the performance of the model for the specific outcome. The estimated and 95% confidence intervals for area under the curve (AUC) were also computed using the Delong method. The robustness of each model was computed by comparing AUC of the model with its single covariates and improvement of the model over the single covariates is reported (significance of improvement p<0.01).

The following tables summarize the results of the univariate analysis performed for the different outcomes.

TABLE 4
Overview of the performance of different markers as infection markers (all sepsis versus SIRS)

| | | | AUC | AUC | missing | | | | |
|---|---|---|---|---|---|---|---|---|---|
| | | Median | (lower | (upper | value | | | fold | assay - |
| Marker | assay.name | AUC | CI) | CI) | rate | median.control | median.case | change | CV |
| PRTN3 | PRTN3_MC508_TrY6_Br1_Ma1 | 0.76 | 0.68 | 0.84 | 2% | 9.3E−03 | 1.9E−02 | 2.0 | 15% |
| S100A9 | S100A9_MC408_TrY10_Br1_Ma1 | 0.68 | 0.59 | 0.77 | 2% | 3.0E−02 | 4.9E−02 | 1.6 | 35% |
| ILR2 | ILR2_MC479_TrY5_Br4_Ma1 | 0.64 | 0.55 | 0.74 | 2% | 8.8E−03 | 1.0E−02 | 1.2 | 24% |
| PCT (ng/ml) | PCT (ng/ml) | 0.67 | 0.59 | 0.76 | 2% | 2.0E−01 | 5.9E−01 | 3.0 | #N/A |
| S100A9 | S100A9_MC407_TrY10_Br1_Ma1 | 0.67 | 0.58 | 0.76 | 1% | 4.2E−02 | 6.4E−02 | 1.5 | 24% |
| wbc | donor_white_blood_cell_count | 0.67 | 0.57 | 0.76 | 1% | 1.1E+01 | 1.4E+01 | 1.2 | #N/A |
| ILR2 | ILR2_MC479_TrY7_Br4_Ma1 | 0.60 | 0.50 | 0.70 | 6% | 9.9E−03 | 1.2E−02 | 1.2 | 23% |
| S100A8 | S100A8_MC517_TrY6_Br1_Ma1 | 0.66 | 0.57 | 0.75 | 2% | 6.2E−02 | 9.1E−02 | 1.5 | 26% |
| ILR2 | ILR2_MC477_TrY8_Br4_Ma2 | 0.60 | 0.51 | 0.70 | 1% | 5.7E−03 | 6.4E−03 | 1.1 | 16% |
| PTX3 | PTX3_MC246_TrY6_Br4_Ma1 | 0.65 | 0.56 | 0.75 | 5% | 4.1E−03 | 6.5E−03 | 1.6 | 25% |
| PTX3 | PTX3_MC246_TrY8_Br4_Ma1 | 0.64 | 0.54 | 0.73 | 6% | 4.1E−03 | 5.9E−03 | 1.5 | 28% |
| ATF6 | ATF6_MC348_TrY7_Br4_Ma2 | 0.63 | 0.54 | 0.72 | 3% | 2.4E−02 | 3.1E−02 | 1.3 | 16% |
| LUM | LUM_MC396_TrY9_Br1_Ma1 | 0.63 | 0.54 | 0.72 | 1% | 7.0E−01 | 5.7E−01 | 1.2 | 19% |
| CRP | CRP_MC144_TrY3_Br4_Ma1 | 0.63 | 0.54 | 0.72 | 0% | 3.2E+01 | 4.0E+01 | 1.3 | 12% |
| CRP | CRP_MC144_TrY6_Br4_Ma1 | 0.62 | 0.53 | 0.71 | 0% | 3.2E+01 | 4.2E+01 | 1.3 | 12% |
| CST3 | CST3_MC089_TrY9_Br4_Ma2 | 0.62 | 0.53 | 0.71 | 2% | 3.9E−01 | 4.6E−01 | 1.2 | 7% |
| CRP | CRP_MC144_TrY6_Br1_Ma1 | 0.62 | 0.53 | 0.71 | 1% | 6.0E+00 | 8.3E+00 | 1.4 | 13% |
| LUM | LUM_MC398_TrY6_Br1_Ma1 | 0.62 | 0.53 | 0.71 | 1% | 3.9E−01 | 3.1E−01 | 1.3 | 12% |
| CRP | CRP_MC144_TrY3_Br1_Ma1 | 0.62 | 0.53 | 0.71 | 1% | 6.3E+00 | 8.6E+00 | 1.4 | 13% |
| TIMP1 | TIMP1_MC251_TrY7_Br4_Ma2 | 0.62 | 0.53 | 0.71 | 2% | 7.0E−02 | 9.1E−02 | 1.3 | 7% |
| CRP | CRP_MC145_TrY6_Br1_Ma1 | 0.62 | 0.53 | 0.71 | 1% | 4.7E+00 | 6.7E+00 | 1.4 | 15% |
| ATF6 | ATF6_MC349_TrY3_Br4_Ma2 | 0.62 | 0.52 | 0.71 | 2% | 1.9E−02 | 2.3E−02 | 1.2 | 14% |
| CRP | CRP_MC145_TrY8_Br1_Ma1 | 0.62 | 0.53 | 0.71 | 1% | 4.5E+00 | 6.3E+00 | 1.4 | 17% |
| TIMP1 | TIMP1_MC251_TrY6_Br4_Ma2 | 0.62 | 0.53 | 0.71 | 2% | 7.0E−02 | 9.2E−02 | 1.3 | 8% |
| TIMP1 | TIMP1_MC250_TrY7_Br4_Ma2 | 0.62 | 0.53 | 0.71 | 1% | 7.4E−02 | 9.0E−02 | 1.2 | 16% |
| TIMP1 | TIMP1_MC250_TrY8_Br4_Ma2 | 0.61 | 0.52 | 0.70 | 1% | 7.9E−02 | 9.6E−02 | 1.2 | 12% |
| LBP | LBP_MC015_TrY2_Br4_Ma1 | 0.61 | 0.52 | 0.70 | 0% | 2.9E+00 | 3.3E+00 | 1.2 | 8% |
| SAA | SAA_MC410_TrY8_Br1_Ma1 | 0.61 | 0.52 | 0.70 | 1% | 6.9E+00 | 1.3E+01 | 1.9 | 16% |
| LBP | LBP_MC015_TrY6_Br4_Ma1 | 0.61 | 0.52 | 0.70 | 0% | 2.8E+00 | 3.4E+00 | 1.2 | 7% |
| SAA | SAA_MC410_TrY9_Br1_Ma1 | 0.61 | 0.51 | 0.70 | 1% | 5.6E+00 | 1.0E+01 | 1.8 | 19% |
| DBI | DBI_MC340_TrY6_Br4_Ma2 | 0.61 | 0.51 | 0.70 | 2% | 1.5E−02 | 1.8E−02 | 1.1 | 19% |
| LUM | LUM_MC398_TrY8_Br4_Ma1 | 0.61 | 0.52 | 0.70 | 0% | 2.3E+00 | 1.8E+00 | 1.2 | 11% |
| CRP | CRP_conc_ug_ml | 0.61 | 0.51 | 0.70 | 4% | 7.0E+01 | 8.8E+01 | 1.3 | #N/A |
| LBP | LBP_MC015_TrY2_Br1_Ma1 | 0.61 | 0.51 | 0.70 | 1% | 4.5E−01 | 5.2E−01 | 1.2 | 13% |
| LUM | LUM_MC398_TrY9_Br4_Ma1 | 0.60 | 0.51 | 0.69 | 1% | 2.2E+00 | 1.8E+00 | 1.2 | 10% |
| ATF6 | ATF6_MC349_TrY9_Br4_Ma2 | 0.60 | 0.51 | 0.69 | 2% | 1.6E−02 | 2.0E−02 | 1.2 | 21% |
| LBP | LBP_MC014_TrY4_Br1_Ma1 | 0.60 | 0.51 | 0.69 | 1% | 5.2E−01 | 6.0E−01 | 1.2 | 17% |
| LBP | LBP_MC015_TrY6_Br4_Ma2 | 0.60 | 0.51 | 0.69 | 1% | 2.9E+00 | 3.3E+00 | 1.2 | 13% |
| B4GALT1 | B4GALT1_MC675_TrY4_Br1_Ma1 | 0.60 | 0.51 | 0.70 | 1% | 6.9E−03 | 9.4E−03 | 1.4 | 15% |
| LBP | LBP_MC014_TrY6_Br1_Ma1 | 0.60 | 0.51 | 0.69 | 1% | 5.5E−01 | 6.0E−01 | 1.1 | 16% |
| LBP | LBP_MC015_TrY8_Br1_Ma1 | 0.60 | 0.51 | 0.69 | 1% | 4.5E−01 | 5.1E−01 | 1.1 | 11% |
| LBP | LBP_MC015_TrY8_Br4_Ma2 | 0.60 | 0.51 | 0.69 | 1% | 2.7E+00 | 3.3E+00 | 1.2 | 7% |
| ICAM1 | ICAM1_MC023_TrY11_Br4_Ma1 | 0.60 | 0.50 | 0.69 | 0% | 3.8E−02 | 4.6E−02 | 1.2 | 9% |
| ICAM1 | ICAM1_MC022_TrY10_Br4_Ma1 | 0.60 | 0.50 | 0.69 | 0% | 7.7E−02 | 8.9E−02 | 1.2 | 14% |

TABLE 5
Overview of the performance of different markers as mild infection markers (mild sepsis versus SIRS)

| | | | AUC | AUC | missing | | | | |
|---|---|---|---|---|---|---|---|---|---|
| | | Median | (lower | (upper | value | | | fold | assay - |
| Marker | assay.name | AUC | CI) | CI) | rate | median.control | median.case | change | CV |
| PRTN3 | PRTN3_MC508_TrY6_Br1_Ma1 | 0.75 | 0.65 | 0.84 | 3% | 9.3E−03 | 1.8E−02 | 1.9 | 15% |
| EXT2 | EXT2_MC451_TrY6_Br4_Ma2 | 0.69 | 0.59 | 0.79 | 3% | 5.4E−03 | 3.5E−03 | 1.5 | 22% |
| S100A9 | S100A9_MC408_TrY10_Br1_Ma1 | 0.68 | 0.58 | 0.78 | 2% | 3.0E−02 | 4.8E−02 | 1.6 | 35% |
| S100A9 | S100A9_MC407_TrY10_Br1_Ma1 | 0.67 | 0.57 | 0.78 | 1% | 4.2E−02 | 6.5E−02 | 1.5 | 24% |
| SAA | SAA_MC410_TrY8_Br1_Ma1 | 0.66 | 0.56 | 0.77 | 1% | 6.9E+00 | 1.8E+01 | 2.6 | 16% |
| EXT2 | EXT2_MC451_TrY5_Br4_Ma2 | 0.66 | 0.56 | 0.77 | 5% | 4.9E−03 | 3.5E−03 | 1.4 | 23% |
| S100A8 | S100A8_MC517_TrY6_Br1_Ma1 | 0.66 | 0.56 | 0.77 | 2% | 6.2E−02 | 8.9E−02 | 1.4 | 26% |
| SAA | SAA_MC410_TrY9_Br1_Ma1 | 0.66 | 0.56 | 0.77 | 1% | 5.6E+00 | 1.2E+01 | 2.2 | 19% |
| CRP | CRP_MC144_TrY3_Br4_Ma1 | 0.66 | 0.56 | 0.76 | 0% | 3.2E+01 | 4.4E+01 | 1.4 | 12% |
| CRP | CRP_MC144_TrY6_Br4_Ma1 | 0.66 | 0.55 | 0.76 | 0% | 3.2E+01 | 4.5E+01 | 1.4 | 12% |
| CRP | CRP_MC145_TrY6_Br1_Ma1 | 0.66 | 0.55 | 0.76 | 1% | 4.7E+00 | 7.2E+00 | 1.6 | 15% |
| CRP | CRP_MC144_TrY6_Br1_Ma1 | 0.65 | 0.55 | 0.76 | 1% | 6.0E+00 | 9.1E+00 | 1.5 | 13% |
| CRP | CRP_MC145_TrY8_Br1_Ma1 | 0.65 | 0.55 | 0.76 | 1% | 4.5E+00 | 6.9E+00 | 1.5 | 17% |
| wbc | donor_white_blood_cell_count | 0.65 | 0.55 | 0.76 | 0% | 1.1E+01 | 1.4E+01 | 1.2 | #N/A |
| LUM | LUM_MC396_TrY9_Br1_Ma1 | 0.65 | 0.55 | 0.76 | 1% | 7.0E−01 | 5.4E−01 | 1.3 | 19% |
| CRP | CRP_MC144_TrY3_Br1_Ma1 | 0.65 | 0.55 | 0.76 | 1% | 6.3E+00 | 9.2E+00 | 1.5 | 13% |
| LUM | LUM_MC398_TrY6_Br1_Ma1 | 0.65 | 0.54 | 0.76 | 1% | 3.9E−01 | 3.1E−01 | 1.3 | 12% |
| ATF6 | ATF6_MC348_TrY7_Br4_Ma2 | 0.65 | 0.54 | 0.75 | 5% | 2.4E−02 | 3.0E−02 | 1.3 | 16% |
| LUM | LUM_MC398_TrY8_Br4_Ma1 | 0.63 | 0.53 | 0.74 | 0% | 2.3E+00 | 1.7E+00 | 1.3 | 11% |
| LUM | LUM_MC398_TrY9_Br4_Ma1 | 0.63 | 0.52 | 0.74 | 1% | 2.2E+00 | 1.8E+00 | 1.2 | 10% |
| FBLN1 | FBLN1_MC039_TrY8_Br4_Ma2 | 0.63 | 0.52 | 0.74 | 0% | 3.6E+00 | 2.8E+00 | 1.3 | 12% |
| PSMA3 | PSMA3_MC510_TrY5_Br4_Ma1 | 0.63 | 0.52 | 0.73 | 1% | 6.4E−03 | 4.9E−03 | 1.3 | 13% |
| FBLN1 | FBLN1_MC039_TrY9_Br4_Ma2 | 0.63 | 0.52 | 0.73 | 1% | 4.0E+00 | 2.9E+00 | 1.4 | 12% |
| FGL1 | FGL1_MC379_TrY8_Br4_Ma1 | 0.62 | 0.52 | 0.73 | 2% | 3.0E−01 | 4.0E−01 | 1.3 | 9% |
| CRP | CRP_conc_ug_ml | 0.62 | 0.51 | 0.73 | 4% | 7.0E+01 | 8.8E+01 | 1.3 | #N/A |
| SAA | SAA_MC411_TrY2_Br1_Ma1 | 0.62 | 0.51 | 0.73 | 12% | 3.8E+00 | 6.3E+00 | 1.7 | 10% |
| LBP | LBP_MC015_TrY2_Br4_Ma1 | 0.62 | 0.51 | 0.72 | 0% | 2.9E+00 | 3.4E+00 | 1.2 | 8% |
| ATF6 | ATF6_MC349_TrY3_Br4_Ma2 | 0.62 | 0.51 | 0.73 | 4% | 1.9E−02 | 2.3E−02 | 1.2 | 14% |
| ATF6 | ATF6_MC349_TrY9_Br4_Ma2 | 0.62 | 0.51 | 0.72 | 3% | 1.6E−02 | 2.0E−02 | 1.2 | 21% |
| LBP | LBP_MC015_TrY6_Br4_Ma1 | 0.61 | 0.51 | 0.72 | 0% | 2.8E+00 | 3.3E+00 | 1.2 | 7% |
| LBP | LBP_MC015_TrY2_Br1_Ma1 | 0.61 | 0.51 | 0.72 | 1% | 4.5E−01 | 5.4E−01 | 1.2 | 13% |
| PSMA3 | PSMA3_MC510_TrY6_Br4_Ma1 | 0.61 | 0.51 | 0.72 | 0% | 6.0E−03 | 4.8E−03 | 1.3 | 11% |
| Heart rate | donor_Heart_rate_bpm | 0.61 | 0.50 | 0.72 | 1% | 1.0E+02 | 9.8E+01 | 1.0 | #N/A |
| CST3 | CST3_MC090_TrY8_Br1_Ma1 | 0.61 | 0.50 | 0.72 | 1% | 2.5E−02 | 2.0E−02 | 1.2 | 15% |

TABLE 6
Overview of the performance of different markers as bacteraemia markers (blood culture positive versus blood culture negative)

| | | | AUC | AUC | missing | | | | |
|---|---|---|---|---|---|---|---|---|---|
| | | Median | (lower | (upper | value | | | fold | assay - |
| Marker | assay.name | AUC | CI) | CI) | rate | median.control | median.case | change | CV |
| PCT (ng/ml) | PCT (ng/ml) | 0.77 | 0.68 | 0.86 | 2% | 2.8E−01 | 2.9E+00 | 10.5 | #N/A |
| PTX3 | PTX3_MC246_TrY6_Br4_Ma1 | 0.73 | 0.63 | 0.83 | 5% | 4.6E−03 | 1.4E−02 | 3.0 | 25% |
| PTX3 | PTX3_MC246_TrY8_Br4_Ma1 | 0.72 | 0.62 | 0.82 | 6% | 4.6E−03 | 1.3E−02 | 2.8 | 28% |
| VCAM1 | VCAM1_MC029_TrY9_Br4_Ma1 | 0.71 | 0.61 | 0.81 | 1% | 1.2E−01 | 2.0E−01 | 1.7 | 12% |
| B4GALT1 | B4GALT1_MC675_TrY5_Br1_Ma1 | 0.70 | 0.60 | 0.81 | 1% | 7.8E−03 | 1.2E−02 | 1.6 | 16% |
| B4GALT1 | B4GALT1_MC675_TrY4_Br1_Ma1 | 0.70 | 0.60 | 0.81 | 1% | 8.1E−03 | 1.3E−02 | 1.7 | 15% |
| CST3 | CST3_MC089_TrY9_Br4_Ma2 | 0.70 | 0.59 | 0.81 | 2% | 4.0E−01 | 6.3E−01 | 1.6 | 7% |
| PRTN3 | PRTN3_MC508_TrY6_Br1_Ma1 | 0.70 | 0.60 | 0.80 | 2% | 1.3E−02 | 2.3E−02 | 1.8 | 15% |
| ICAM1 | ICAM1_MC022_TrY10_Br4_Ma1 | 0.69 | 0.59 | 0.80 | 0% | 8.1E−02 | 1.2E−01 | 1.5 | 14% |
| VCAM1 | VCAM1_MC029_TrY10_Br4_Ma1 | 0.68 | 0.58 | 0.79 | 1% | 1.2E−01 | 1.9E−01 | 1.5 | 12% |
| PTPRG | PTPRG_MC512_TrY5_Br4_Ma1 | 0.68 | 0.57 | 0.78 | 0% | 3.6E−02 | 4.5E−02 | 1.2 | 9% |
| ICAM1 | ICAM1_MC023_TrY11_Br4_Ma1 | 0.68 | 0.57 | 0.78 | 0% | 4.0E−02 | 5.7E−02 | 1.4 | 9% |
| LBP | LBP_MC015_TrY2_Br4_Ma1 | 0.67 | 0.57 | 0.77 | 0% | 2.9E+00 | 3.8E+00 | 1.3 | 8% |
| LBP | LBP_MC015_TrY8_Br4_Ma2 | 0.66 | 0.56 | 0.77 | 1% | 2.8E+00 | 3.7E+00 | 1.4 | 7% |
| PTPRG | PTPRG_MC512_TrY6_Br4_Ma1 | 0.66 | 0.56 | 0.76 | 0% | 3.8E−02 | 4.4E−02 | 1.2 | 16% |
| LBP | LBP_MC015_TrY6_Br4_Ma1 | 0.66 | 0.56 | 0.76 | 0% | 2.8E+00 | 3.8E+00 | 1.3 | 7% |
| CHI3L1 | CHI3L1_MC365_TrY9_Br4_Ma1 | 0.66 | 0.55 | 0.77 | 1% | 1.7E−02 | 3.7E−02 | 2.1 | 17% |
| LBP | LBP_MC015_TrY8_Br1_Ma1 | 0.66 | 0.56 | 0.76 | 1% | 4.9E−01 | 6.5E−01 | 1.3 | 11% |
| CHI3L1 | CHI3L1_MC364_TrY8_Br4_Ma1 | 0.66 | 0.55 | 0.76 | 1% | 2.7E−02 | 4.8E−02 | 1.8 | 16% |
| ICAM1 | ICAM1_MC023_TrY7_Br4_Ma1 | 0.66 | 0.55 | 0.76 | 1% | 3.9E−02 | 5.4E−02 | 1.4 | 19% |
| LBP | LBP_MC014_TrY4_Br1_Ma1 | 0.65 | 0.55 | 0.75 | 1% | 5.3E−01 | 7.2E−01 | 1.4 | 17% |
| LBP | LBP_MC015_TrY6_Br4_Ma2 | 0.65 | 0.55 | 0.75 | 1% | 2.9E+00 | 3.7E+00 | 1.3 | 13% |
| GOLM1 | GOLM1_MC067_TrY2_Br4_Ma2 | 0.65 | 0.55 | 0.75 | 2% | 2.1E−02 | 3.2E−02 | 1.5 | 19% |
| TIMP1 | TIMP1_MC251_TrY7_Br4_Ma2 | 0.65 | 0.54 | 0.77 | 2% | 8.1E−02 | 1.1E−01 | 1.4 | 7% |
| CRP | CRP_MC144_TrY3_Br4_Ma1 | 0.65 | 0.55 | 0.75 | 0% | 3.5E+01 | 5.0E+01 | 1.4 | 12% |
| LBP | LBP_MC015_TrY2_Br1_Ma1 | 0.65 | 0.55 | 0.75 | 1% | 4.5E−01 | 5.8E−01 | 1.3 | 13% |
| ILR2 | ILR2_MC479_TrY5_Br4_Ma1 | 0.65 | 0.54 | 0.76 | 2% | 9.7E−03 | 1.2E−02 | 1.2 | 23% |
| ATF6 | ATF6_MC348_TrY7_Br4_Ma2 | 0.65 | 0.54 | 0.76 | 3% | 2.7E−02 | 3.6E−02 | 1.3 | 16% |
| CRP | CRP_MC144_TrY6_Br4_Ma1 | 0.65 | 0.54 | 0.75 | 0% | 3.6E+01 | 4.8E+01 | 1.4 | 12% |
| CRP | CRP_MC145_TrY6_Br1_Ma1 | 0.64 | 0.54 | 0.75 | 1% | 5.1E+00 | 8.0E+00 | 1.6 | 15% |
| LBP | LBP_MC014_TrY6_Br1_Ma1 | 0.64 | 0.54 | 0.74 | 1% | 5.4E−01 | 6.7E−01 | 1.2 | 16% |
| TIMP1 | TIMP1_MC251_TrY6_Br4_Ma2 | 0.64 | 0.53 | 0.76 | 2% | 8.2E−02 | 1.2E−01 | 1.4 | 8% |
| CSF1 | CSF1_MC368_TrY10_Br4_Ma1 | 0.64 | 0.53 | 0.75 | 4% | 1.6E−02 | 2.0E−02 | 1.3 | 27% |
| ILR2 | ILR2_MC479_TrY7_Br4_Ma1 | 0.64 | 0.53 | 0.75 | 6% | 1.0E−02 | 1.3E−02 | 1.2 | 24% |
| TIMP1 | TIMP1_MC250_TrY8_Br4_Ma2 | 0.64 | 0.52 | 0.76 | 1% | 8.4E−02 | 1.2E−01 | 1.4 | 12% |
| EXT2 | EXT2_MC452_TrY5_Br4_Ma2 | 0.64 | 0.52 | 0.76 | 19% | 5.5E−03 | 7.8E−03 | 1.4 | 16% |
| CRP | CRP_MC145_TrY8_Br1_Ma1 | 0.64 | 0.54 | 0.74 | 1% | 5.0E+00 | 7.6E+00 | 1.5 | 17% |
| ATF6 | ATF6_MC348_TrY8_Br4_Ma2 | 0.64 | 0.53 | 0.75 | 4% | 2.9E−02 | 3.5E−02 | 1.2 | 21% |
| CRP | CRP_MC144_TrY6_Br1_Ma1 | 0.64 | 0.53 | 0.74 | 1% | 6.4E+00 | 1.0E+01 | 1.6 | 13% |
| CRP | CRP_MC144_TrY3_Br1_Ma1 | 0.64 | 0.53 | 0.74 | 1% | 6.7E+00 | 1.1E+01 | 1.7 | 13% |
| CHI3L1 | CHI3L1_MC364_TrY7_Br4_Ma1 | 0.63 | 0.52 | 0.74 | 1% | 3.1E−02 | 5.1E−02 | 1.6 | 29% |
| GOLM1 | GOLM1_MC067_TrY6_Br4_Ma2 | 0.63 | 0.52 | 0.74 | 2% | 2.2E−02 | 3.2E−02 | 1.5 | 24% |
| TIMP1 | TIMP1_MC250_TrY7_Br4_Ma2 | 0.63 | 0.51 | 0.75 | 1% | 8.1E−02 | 1.1E−01 | 1.4 | 16% |
| PTPRG | PTPRG_MC514_TrY8_Br4_Ma2 | 0.63 | 0.52 | 0.74 | 1% | 3.5E−02 | 4.1E−02 | 1.2 | 15% |
| MRC1 | MRC1_MC102_TrY9_Br4_Ma2 | 0.63 | 0.52 | 0.73 | 1% | 3.9E−02 | 4.7E−02 | 1.2 | 6% |
| MRC1 | MRC1_MC102_TrY8_Br4_Ma2 | 0.62 | 0.52 | 0.73 | 2% | 3.7E−02 | 4.7E−02 | 1.3 | 12% |
| ATF6 | ATF6_MC349_TrY9_Br4_Ma2 | 0.62 | 0.51 | 0.73 | 2% | 1.8E−02 | 2.4E−02 | 1.3 | 21% |
| GOLM1 | GOLM1_MC068_TrY7_Br4_Ma2 | 0.62 | 0.51 | 0.72 | 2% | 8.0E−03 | 1.1E−02 | 1.4 | 29% |
| ATF6 | ATF6_MC349_TrY3_Br4_Ma2 | 0.62 | 0.51 | 0.72 | 2% | 2.1E−02 | 2.3E−02 | 1.1 | 14% |

TABLE 7
Overview of the performance of different markers as organ failure markers (organ failure positive versus organ failure negative)

| | | | AUC | AUC | missing | | | | |
|---|---|---|---|---|---|---|---|---|---|
| | | Median | (lower | (upper | value | | | fold | assay - |
| Marker | assay.name | AUC | CI) | CI) | rate | median.control | median.case | change | CV |
| EXT2 | EXT2_MC451_TrY5_Br4_Ma2 | 0.66 | 0.58 | 0.75 | 4% | 3.5E−03 | 5.8E−03 | 1.6 | 23% |
| PTX3 | PTX3_MC246_TrY6_Br4_Ma1 | 0.65 | 0.57 | 0.74 | 5% | 4.3E−03 | 8.0E−03 | 1.8 | 25% |
| EXT2 | EXT2_MC452_TrY6_Br4_Ma2 | 0.65 | 0.56 | 0.74 | 14% | 4.3E−03 | 6.8E−03 | 1.6 | 28% |
| EXT2 | EXT2_MC451_TrY6_Br4_Ma2 | 0.65 | 0.56 | 0.73 | 3% | 3.8E−03 | 5.7E−03 | 1.5 | 22% |
| CST3 | CST3_MC090_TrY8_Br1_Ma1 | 0.64 | 0.55 | 0.72 | 1% | 2.2E−02 | 2.9E−02 | 1.3 | 15% |
| CHI3L1 | CHI3L1_MC364_TrY8_Br4_Ma1 | 0.64 | 0.55 | 0.72 | 1% | 2.1E−02 | 3.7E−02 | 1.8 | 16% |
| CST3 | CST3_MC090_TrY9_Br1_Ma1 | 0.64 | 0.55 | 0.72 | 1% | 2.2E−02 | 2.7E−02 | 1.2 | 21% |
| CHI3L1 | CHI3L1_MC365_TrY9_Br4_Ma1 | 0.63 | 0.55 | 0.72 | 1% | 1.6E−02 | 2.8E−02 | 1.8 | 17% |
| CST3 | CST3_MC090_TrY9_Br4_Ma2 | 0.63 | 0.55 | 0.72 | 1% | 1.1E−01 | 1.4E−01 | 1.3 | 18% |
| EXT2 | EXT2_MC452_TrY5_Br4_Ma2 | 0.63 | 0.54 | 0.73 | 19% | 5.1E−03 | 6.6E−03 | 1.3 | 16% |
| CST3 | CST3_MC090_TrY8_Br4_Ma2 | 0.63 | 0.54 | 0.71 | 1% | 1.1E−01 | 1.5E−01 | 1.3 | 15% |
| CST3 | CST3_MC089_TrY9_Br4_Ma2 | 0.62 | 0.54 | 0.71 | 2% | 4.0E−01 | 4.6E−01 | 1.2 | 7% |
| CHI3L1 | CHI3L1_MC364_TrY7_Br4_Ma1 | 0.62 | 0.54 | 0.71 | 1% | 2.9E−02 | 4.6E−02 | 1.6 | 29% |
| ILR2 | ILR2_MC479_TrY5_Br4_Ma1 | 0.62 | 0.53 | 0.70 | 2% | 9.5E−03 | 1.1E−02 | 1.1 | 16% |
| MRC1 | MRC1_MC102_TrY9_Br4_Ma2 | 0.62 | 0.53 | 0.70 | 1% | 3.8E−02 | 4.6E−02 | 1.2 | 6% |
| PSMA3 | PSMA3_MC510_TrY5_Br4_Ma1 | 0.62 | 0.53 | 0.70 | 1% | 5.1E−03 | 6.7E−03 | 1.3 | 13% |
| PCT (ng/ml) | PCT (ng/ml) | 0.62 | 0.53 | 0.70 | 2% | 2.5E−01 | 5.4E−01 | 2.2 | #N/A |
| TIMP1 | TIMP1_MC251_TrY7_Br4_Ma2 | 0.62 | 0.53 | 0.70 | 2% | 7.5E−02 | 9.6E−02 | 1.3 | 7% |
| ILR2 | ILR2_MC477_TrY8_Br4_Ma2 | 0.61 | 0.53 | 0.70 | 1% | 5.8E−03 | 6.5E−03 | 1.1 | 24% |
| TIMP1 | TIMP1_MC251_TrY6_Br4_Ma2 | 0.61 | 0.53 | 0.70 | 2% | 7.2E−02 | 9.4E−02 | 1.3 | 8% |
| PTX3 | PTX3_MC246_TrY8_Br4_Ma1 | 0.61 | 0.52 | 0.70 | 6% | 4.6E−03 | 6.7E−03 | 1.5 | 28% |
| MRC1 | MRC1_MC102_TrY8_Br4_Ma2 | 0.61 | 0.53 | 0.70 | 2% | 3.6E−02 | 4.6E−02 | 1.3 | 12% |
| Heart rate | donor_Heart_rate_bpm | 0.61 | 0.52 | 0.69 | 1% | 9.8E+01 | 1.0E+02 | 1.0 | #N/A |
| PSMA3 | PSMA3_MC510_TrY6_Br4_Ma1 | 0.61 | 0.52 | 0.69 | 0% | 4.7E−03 | 6.6E−03 | 1.4 | 11% |
| TIMP1 | TIMP1_MC250_TrY8_Br4_Ma2 | 0.61 | 0.52 | 0.69 | 1% | 8.1E−02 | 1.0E−01 | 1.3 | 12% |
| B4GALT1 | B4GALT1_MC675_TrY5_Br1_Ma1 | 0.60 | 0.52 | 0.69 | 1% | 7.7E−03 | 9.8E−03 | 1.3 | 16% |
| SAA | SAA_MC411_TrY2_Br1_Ma1 | 0.60 | 0.51 | 0.69 | 10% | 5.6E+00 | 3.6E+00 | 1.6 | 10% |
| B4GALT1 | B4GALT1_MC675_TrY4_Br1_Ma1 | 0.60 | 0.51 | 0.69 | 1% | 7.7E−03 | 1.1E−02 | 1.4 | 15% |
| NID1 | NID1_MC044_TrY9_Br1_Ma1 | 0.60 | 0.51 | 0.69 | 2% | 5.3E−03 | 6.5E−03 | 1.2 | 18% |
| FGL1 | FGL1_MC379_TrY8_Br4_Ma1 | 0.60 | 0.51 | 0.68 | 2% | 3.8E−01 | 2.8E−01 | 1.4 | 9% |
| TIMP1 | TIMP1_MC250_TrY7_Br4_Ma2 | 0.59 | 0.51 | 0.68 | 1% | 7.9E−02 | 9.2E−02 | 1.2 | 16% |
| DBI | DBI_MC340_TrY6_Br4_Ma2 | 0.59 | 0.50 | 0.68 | 2% | 1.6E−02 | 1.8E−02 | 1.1 | 19% |
| SAA | SAA_MC410_TrY8_Br1_Ma1 | 0.59 | 0.50 | 0.68 | 1% | 1.2E+01 | 8.0E+00 | 1.6 | 16% |

TABLE 8
Overview of the performance of different markers as prognostic markers (survivors versus non-survivors)

| | | | AUC | AUC | missing | | | | |
|---|---|---|---|---|---|---|---|---|---|
| | | Median | (lower | (upper | value | | | fold | assay - |
| Marker | assay.name | AUC | CI) | CI) | rate | median.control | median.case | change | CV |
| MRC1 | MRC1_MC102_TrY9_Br4_Ma2 | 0.77 | 0.66 | 0.88 | 1% | 3.9E−02 | 6.4E−02 | 1.7 | 6% |
| MRC1 | MRC1_MC102_TrY8_Br4_Ma2 | 0.76 | 0.66 | 0.87 | 2% | 3.7E−02 | 6.3E−02 | 1.7 | 12% |
| GSS | GSS_MC383_TrY6_Br4_Ma1 | 0.71 | 0.60 | 0.83 | 1% | 1.1E−02 | 1.5E−02 | 1.3 | 8% |
| VCAM1 | VCAM1_MC029_TrY9_Br4_Ma1 | 0.71 | 0.58 | 0.84 | 1% | 1.2E−01 | 1.9E−01 | 1.6 | 12% |
| GSS | GSS_MC382_TrY7_Br4_Ma1 | 0.69 | 0.57 | 0.81 | 0% | 1.0E−02 | 1.4E−02 | 1.4 | 17% |
| TIMP1 | TIMP1_MC250_TrY7_Br4_Ma2 | 0.68 | 0.56 | 0.81 | 1% | 8.1E−02 | 1.2E−01 | 1.5 | 16% |
| TIMP1 | TIMP1_MC250_TrY8_Br4_Ma2 | 0.68 | 0.56 | 0.80 | 1% | 8.5E−02 | 1.2E−01 | 1.4 | 12% |
| CHI3L1 | CHI3L1_MC365_TrY9_Br4_Ma1 | 0.68 | 0.55 | 0.80 | 1% | 1.8E−02 | 4.1E−02 | 2.4 | 17% |
| ICAM1 | ICAM1_MC022_TrY10_Br4_Ma1 | 0.68 | 0.55 | 0.80 | 0% | 8.2E−02 | 1.0E−01 | 1.3 | 14% |
| GOLM1 | GOLM1_MC067_TrY2_Br4_Ma2 | 0.67 | 0.53 | 0.82 | 2% | 2.2E−02 | 4.2E−02 | 1.9 | 19% |
| PTPRG | PTPRG_MC514_TrY3_Br4_Ma2 | 0.67 | 0.55 | 0.80 | 2% | 3.6E−02 | 4.3E−02 | 1.2 | 16% |
| GOLM1 | GOLM1_MC067_TrY6_Br4_Ma2 | 0.67 | 0.54 | 0.81 | 2% | 2.3E−02 | 4.1E−02 | 1.8 | 24% |
| PSMA3 | PSMA3_MC510_TrY5_Br4_Ma1 | 0.67 | 0.55 | 0.80 | 1% | 5.9E−03 | 9.9E−03 | 1.7 | 13% |
| VCAM1 | VCAM1_MC029_TrY10_Br4_Ma1 | 0.67 | 0.53 | 0.80 | 1% | 1.3E−01 | 1.7E−01 | 1.4 | 12% |
| B4GALT1 | B4GALT1_MC675_TrY4_Br1_Ma1 | 0.67 | 0.52 | 0.81 | 1% | 8.6E−03 | 1.3E−02 | 1.5 | 15% |
| PSMA3 | PSMA3_MC510_TrY6_Br4_Ma1 | 0.67 | 0.53 | 0.80 | 0% | 5.5E−03 | 8.7E−03 | 1.6 | 11% |
| TIMP1 | TIMP1_MC251_TrY6_Br4_Ma2 | 0.66 | 0.53 | 0.80 | 2% | 8.3E−02 | 1.1E−01 | 1.4 | 8% |
| EXT2 | EXT2_MC452_TrY5_Br4_Ma2 | 0.66 | 0.50 | 0.82 | 19% | 6.0E−03 | 8.7E−03 | 1.5 | 16% |
| TIMP1 | TIMP1_MC251_TrY7_Br4_Ma2 | 0.66 | 0.54 | 0.79 | 2% | 8.3E−02 | 1.1E−01 | 1.4 | 7% |
| GOLM1 | GOLM1_MC068_TrY7_Br4_Ma2 | 0.66 | 0.53 | 0.80 | 2% | 8.2E−03 | 1.3E−02 | 1.6 | 29% |
| CHI3L1 | CHI3L1_MC364_TrY8_Br4_Ma1 | 0.66 | 0.53 | 0.79 | 1% | 2.7E−02 | 5.8E−02 | 2.1 | 16% |
| B4GALT1 | B4GALT1_MC675_TrY5_Br1_Ma1 | 0.65 | 0.51 | 0.80 | 1% | 8.1E−03 | 1.2E−02 | 1.5 | 16% |
| ICAM1 | ICAM1_MC023_TrY11_Br4_Ma1 | 0.65 | 0.52 | 0.79 | 0% | 4.0E−02 | 5.7E−02 | 1.4 | 9% |
| PTPRG | PTPRG_MC512_TrY6_Br4_Ma1 | 0.65 | 0.51 | 0.79 | 0% | 3.9E−02 | 4.6E−02 | 1.2 | 16% |
| CHI3L1 | CHI3L1_MC364_TrY7_Br4_Ma1 | 0.64 | 0.52 | 0.77 | 1% | 3.4E−02 | 5.5E−02 | 1.6 | 29% |
| CST3 | CST3_MC089_TrY9_Br4_Ma2 | 0.64 | 0.50 | 0.78 | 2% | 4.1E−01 | 6.1E−01 | 1.5 | 7% |
| EXT2 | EXT2_MC451_TrY6_Br4_Ma2 | 0.64 | 0.50 | 0.78 | 3% | 4.7E−03 | 6.6E−03 | 1.4 | 22% |
| HSPA8 | HSPA8_MC469_TrY8_Br1_Ma1 | 0.64 | 0.54 | 0.75 | 2% | 1.1E−02 | 1.3E−02 | 1.2 | 15% |
| GPLD1 | GPLD1_MC018_TrY6_Br4_Ma2 | 0.64 | 0.52 | 0.76 | 0% | 5.2E−01 | 4.2E−01 | 1.2 | 9% |
| temp | donor_Temperature_C | 0.64 | 0.51 | 0.77 | 0% | 3.9E+01 | 3.8E+01 | 1.0 | #N/A |
| PCT (ng/ml) | PCT (ng/ml) | 0.64 | 0.51 | 0.76 | 2% | 3.3E−01 | 6.2E−01 | 1.8 | #N/A |
| PTPRG | PTPRG_MC512_TrY5_Br4_Ma1 | 0.64 | 0.50 | 0.78 | 0% | 3.7E−02 | 4.5E−02 | 1.2 | 9% |
| PTX3 | PTX3_MC246_TrY8_Br4_Ma1 | 0.64 | 0.51 | 0.76 | 6% | 4.9E−03 | 8.6E−03 | 1.7 | 28% |
| GPLD1 | GPLD1_MC018_TrY6_Br1_Ma1 | 0.63 | 0.51 | 0.76 | 1% | 1.0E−01 | 7.4E−02 | 1.3 | 12% |
| CAMP | CAMP_MC433_TrY5_Br1_Ma1 | 0.63 | 0.51 | 0.75 | 1% | 2.8E−02 | 2.1E−02 | 1.4 | 11% |
| GPLD1 | GPLD1_MC018_TrY11_Br1_Ma1 | 0.63 | 0.51 | 0.75 | 1% | 1.0E−01 | 8.9E−02 | 1.1 | 16% |

### Example 4

### Proteinase 3 (PRTN3) as New Sepsis Diagnostic Marker

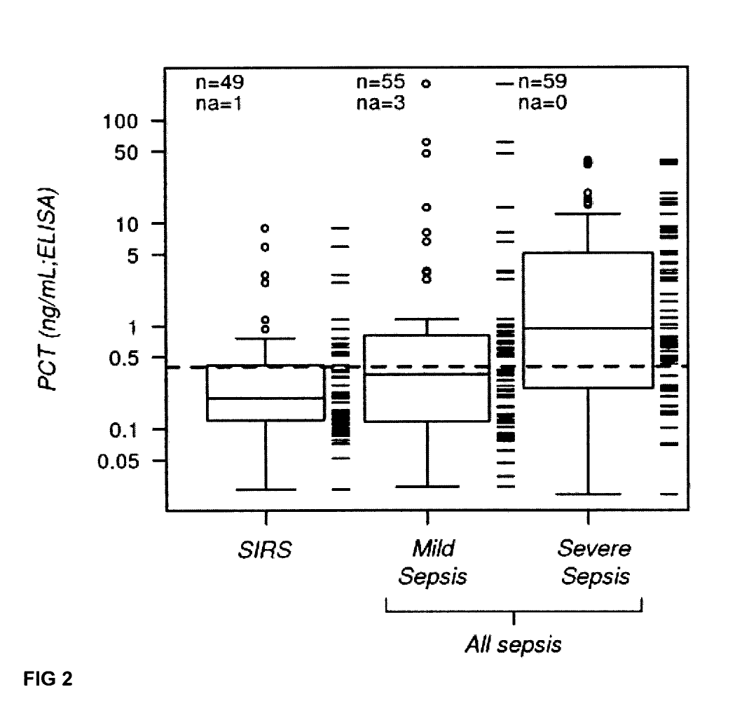

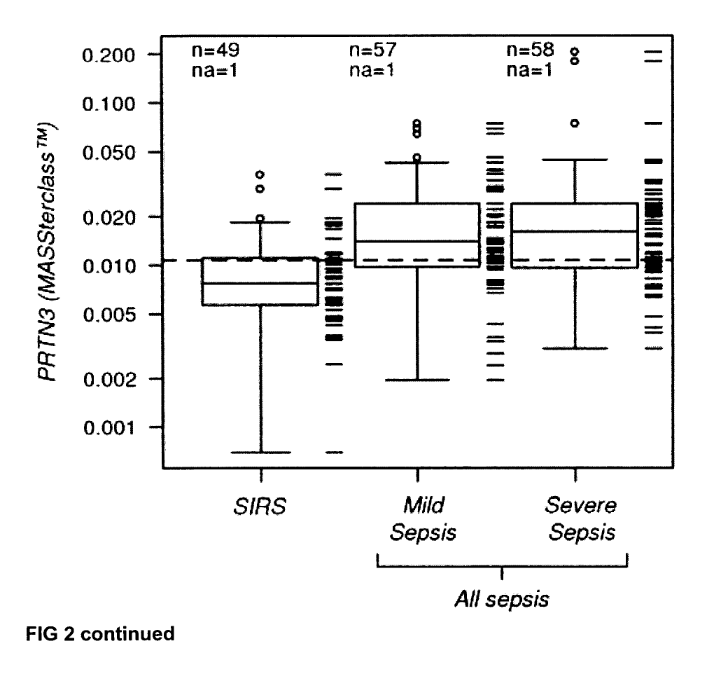

PRTN3 was identified as a particularly promising marker to detect infection in patients with systemic inflammatory response syndrome. The marker showed a diagnostic performance with an AUC of 0.76 (0.68-0.84) significantly better than currently used markers such as procalcitonin (PCT) (Table 4) This diagnostic performance was maintained when only mild sepsis cases were considered, i.e., when sepsis patients with compromised organ function were excluded from the analysis (Table 5). As illustrated in FIG. 2, PRTN3 median levels were 2-fold higher in sepsis patients compared to SIRS patients. However, no difference in levels was observed between mild sepsis and severe sepsis patients (FIG. 2, right panel). This was different to PCT where a stepwise increase in levels was observed going from SIRS to sepsis to severe sepsis patients (FIG. 2, left panel). For PCT, there was a significant overlap between sepsis cases and SIRS controls and this was mainly true for the sepsis patients without organ failure, i.e., mild sepsis (FIG. 2, left panel). The discriminatory power of PCT between SIRS and sepsis patients was largely driven by the severe sepsis patients while PCT had close to no diagnostic performance to detect the mild sepsis cases (AUC=0.58). PRTN3 and PCT had significantly comparable performance to detect the bacteraemia patients, i.e., patients with positive blood culture (Tables 6 and 9). However, PCT failed to detect infection in a background of severe inflammation.

TABLE 9
Overview of the diagnostic performance PRTN3 for sepsis
diagnosis using different definitions of sepsis

| | SIRS versus | SIRS versus | SIRS versus | |
|---|---|---|---|---|
| marker | all SEPSIS | ‘mild’ sepsis | severe sepsis | bacteraemia |
| PRTN3 | 0.76 (0.68-0.84) | 0.75 (0.65-0.84) | 0.78 (0.69-0.85) | 0.70 (0.60-0.80) |
| PCT (ng/ml) | 0.67 (0.59-0.76) | 0.58 (0.47-0.69) | 0.76 (0.66-0.83) | 0.77 (0.68-0.86) |
| WBC | 0.67 (0.57-0.77) | 0.65 (0.55-0.78) | 0.59 (0.48-0.69) | 0.58 (0.47-0.72) |

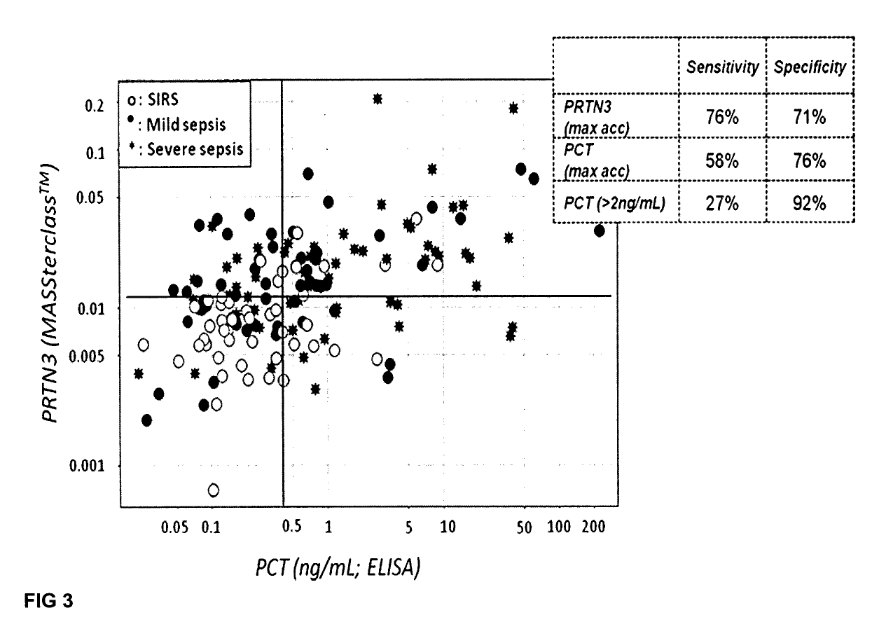

FIG. 3 illustrates the difference in levels between PRTN3 (y-axis) and PCT (x-axis) per patient. Translating AUC numbers to sensitivity and specificity values showed PRTN3 had a significant better sensitivity compared to PCT (FIG. 3). At the cut-off for maximum accuracy PRTN3 reached a sensitivity of 76% combined with a specificity of 71%. PCT showed a maximum combined sensitivity and specificity of 58% and 76% respectively. PCT at its recommended cut-off of 2 ng/mL indeed showed sepsis was very likely (high specificity) but the majority of sepsis cases were missed. FIG. 3 thus illustrates the value of PRTN3 as a biomarker for diagnosing sepsis vs. infection-free SIRS. Furthermore, FIG. 3 underscores the value of PRTN3 for detection of sepsis patients without organ failure, i.e., with mild sepsis.

Looking at PRTN3 in more detail showed no major marker dependencies other than presence of infection. It was further observed that PRTN3 levels were independent of the primary focus of infection: no difference in levels could be observed between patients with respiratory tract infection (RTI), gastro-intestinal tract infection (GTI), urinary tract infection (UTI) or other infectious foci (data not shown). Segregating the sepsis patients based on type of micro-organism, gram positive versus gram negative showed no specificity for either class (data not shown). Also for PCT no relation to type of micro-organism could be observed.

To assess the potential added value of combining PRTN3 and PCT, a logistic regression analysis was performed whereby all sepsis and SIRS patients were used as a training set. The resulting marker combination had a diagnostic performance (AUC=0.77) significantly better than PCT alone. However, the combination formed no improvement over PRTN3 alone in this dataset (Table 10). Table 10 further shows that the largest improvement over PCT was in detecting the mild sepsis cases, i.e., sepsis without organ failure.

TABLE 10
Diagnostic performance of PRTN3, PCT and
the combination of both to detect sepsis

| marker | PRTN3 | PCT (ng/ml) | PRTN3 + PCT | Significance* |
|---|---|---|---|---|
| SIRS versus | 0.76 (0.68-0.84) | 0.67 (0.59-0.76) | 0.77 (0.69-0.85) | P = 0.009 |
| all sepsis | | | | |
| SIRS versus | 0.75 (0.65-0.84) | 0.58 (0.47-0.69) | 0.74 (0.64-0.84) | P = 0.003 |
| mild sepsis | | | | |
| SIRS versus | 0.78 (0.69-0.85) | 0.76 (0.66-0.83) | 0.80 (0.71-0.87) | P = 0.3 |
| severe sepsis | | | | |
| Bacteraemia | 0.70 (0.60-0.80) | 0.77 (0.68-0.86) | 0.73 (0.63-0.82) | P = 0.4 |

*P-value for improvement over PCT

Although no significant added value of combining marker PRTN3 with PCT could be observed over PRTN3 in this dataset, larger cohorts are warranted to truly test this. The different behaviour of the two markers does suggest potential clinical benefit in combining both markers. PRTN3 shows greater sensitivity for detecting infection and has equal performance for mild and severe sepsis cases. PCT on the other hand is especially elevated in patients with severe sepsis and bacteraemia. Both markers were tested in sizeable cohorts to demonstrate their synergistic value.

### Example 5

### MRC1 as Novel Prognostic Markers in Patients with Severe Inflammatory Disease

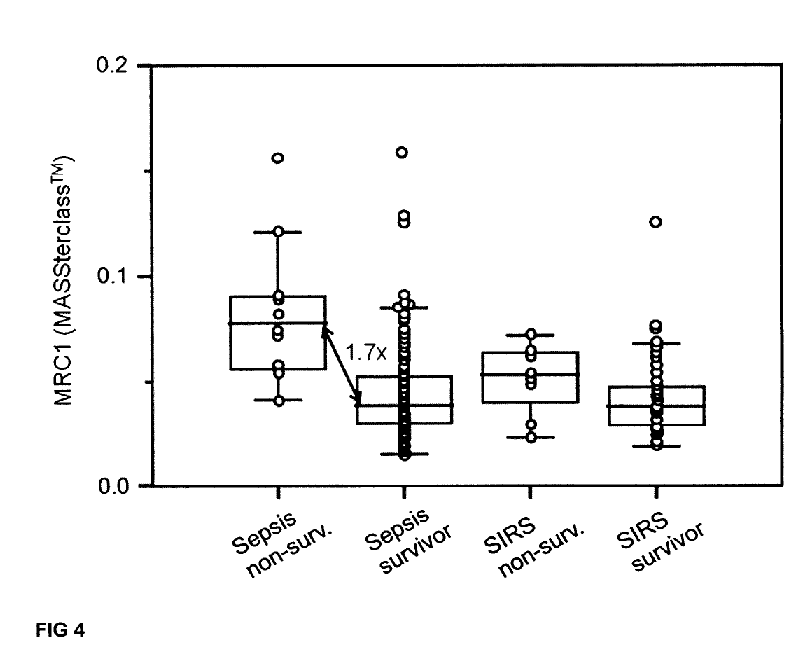

In this cohort of sepsis and SIRS patients, 22 died during the first month of follow-up. These non-survivors were equally distributed over SIRS and sepsis patient groups. All markers were assessed for their performance as prognostic markers to predict mortality within 28 days post sampling. In this cohort, MCR1 showed a particularly better performance (AUC=0.77; 95% CI 0.66-0.88) than all other markers including PCT (Table 8). As shown in FIG. 4, MRC1 median levels were increased in non-survivors compared to survivors both in sepsis patients and in SIRS patients.

### Example 6

### Novel Markers to Detect Organ Failure in Patients with Systemic Inflammatory Disease

Several markers could be identified that showed equal performance to PCT to detect organ failure in patients with systemic inflammatory responses (Tables 7 and 11). Pentraxin-3 (PTX3) and interleukin 1 receptor type II (IL1R2) may be used as severity of disease markers since both showed a better performance to detect severe sepsis patients in patients with SIRS (Tables 7 and 11). Both these markers were indeed elevated in severe sepsis patients. PTX3 and IL1R2 showed no dependence on the type of organ failing.

TABLE 11
Overview of different single markers to detect organ failure

| | | SIRS versus | Sepsis versus |
|---|---|---|---|
| Marker | Organ failure | severe sepsis | severe sepsis |
| EXT2 | 0.66 (0.58-0.75) | 0.52 (0.41-0.63) | 0.74 (0.64-0.82) |
| PTX3 | 0.65 (0.57-0.74) | 0.73 (0.63-0.82) | 0.71 (0.61-0.79) |
| Cystatin C | 0.62 (0.54-0.71) | 0.73 (0.62-0.82) | 0.70 (0.61-0.79) |
| IL1R2 | 0.62 (0.53-0.7) | 0.76 (0.66-0.85) | 0.68 (0.58-0.77) |
| PCT (ng/mL) | 0.62 (0.53-0.7) | 0.76 (0.66-0.85) | 0.66 (0.57-0.75) |

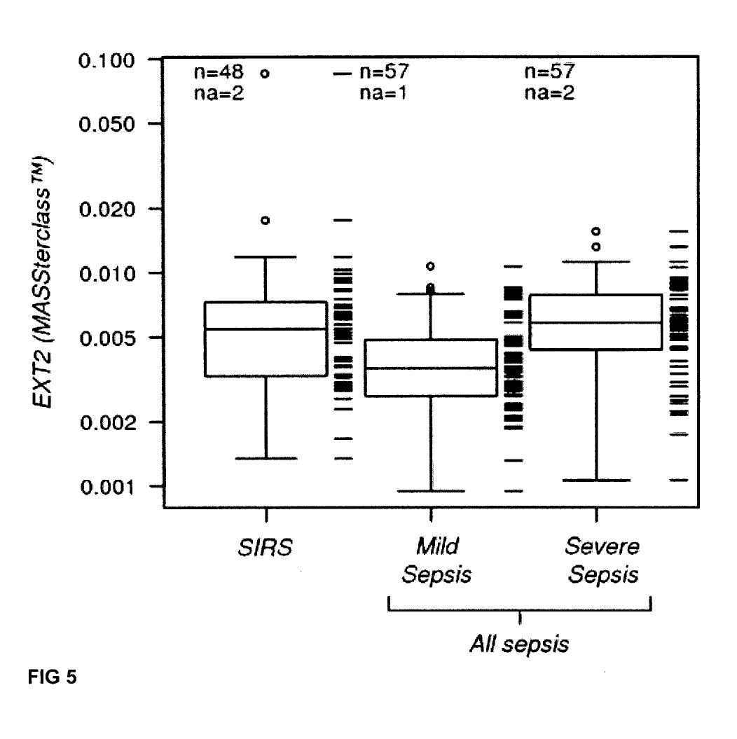

EXT2 showed significantly higher levels in sepsis patients with organ failure compared to sepsis patient without organ failure (FIG. 5 and Table 11). However, this was not observed in SIRS patients (FIG. 5 and Table 11). In fact, SIRS patients seemed to show a large spread of EXT2 levels (FIG. 5). Therefore, EXT2 may be particularly useful to diagnose organ failure in sepsis patients. Except from organ failure, EXT2 showed no other strong relationship with any of the available clinical parameters. Based on its expression levels in sepsis patients, this marker may add value to a marker panel to predict development of severe sepsis.

### Multimarker Models

All markers with stand-alone performance either to detect organ failure or to detect severe sepsis were taken forward in a multivariate logistic regression analysis. This analysis shows that combining a marker with PCT may somewhat improve the performance to detect organ failure or severe sepsis (Table 12).

TABLE 12
Overview of multimarker models to detect organ failure

| | | SIRS versus |
|---|---|---|
| Marker | Organ failure | severe sepsis |
| PCT (ng/mL) | 0.62 (0.53-0.7) | 0.76 (0.66-0.85) |
| Cystatin C | 0.62 (0.54-0.71) | |
| PCT + Cystatin C | 0.64 (0.56-0.73) | |
| IL1R2 | 0.62 (0.53-0.7) | 0.76 (0.66-0.85) |
| PCT + IL1R2 | 0.62 (053-0.71) | 0.79 (0.70-0.86) |
| PTX3 | 0.65 (0.57-0.74) | 0.73 (0.63-0.82) |
| PCT + PTX3 | 0.68 (0.60-0.75) | 0.80 (0.70-0.87) |

### Example 7

### Diagnostic Performance of Marker Combinations

The potential of a marker panel to discriminate sepsis from SIRS was further evaluated in a cohort of 332 plasma samples from a banked, hospital wide database. Patients with suspicion of infection were sampled at time of blood culture, with exclusion of patients with septic shock and patients under treatment of immunosuppressive agents. Final diagnosis and classification as either sepsis or SIRS was done by independent physicians and based on imaging, culture of micro-organisms, antibiotics therapy success and patient presentation. Table 13 summarizes the most important patient characteristics.

TABLE 13
Summary of patient characteristics

| | SIRS (n = 94) | Sepsis (n = 238) |
|---|---|---|
| Age | 56 (18-88) | 60 (17-97) |
| Gender (% male) | 50% | 50% |
| SOFA score | 2 (0-8) | 2 (0-11) |
| % non-survivor | 10% | 5% |
| % bacteraemia | 0 | 33% |
| Focus of infection: | | |
| Respiratory tract | 0 | 22% (n = 53) |
| Urinary tract | 0 | 25% (n = 59) |
| Gastro-intestinal tract | 0 | 18% (n = 43) |
| other | 0 | 35% (n = 83) |
| White blood cell count | 12 (0.2-27) | 14 (0.6-46) |
| (×10⁹ cells/L) | | |
| Body temperature (C.) | 38.8 (34.0-41.0) | 38.7 (35.0-41.0) |
| Heart rate (beats/min) | 100 (60-200) | 100 (44-160) |
| CRP (μg/mL) | 78 (3-275) | 99 (5-422) |
| PCT (μg/mL) | 0.2 (0.02-14.4) | 0.6 (0.02-223) |

Logistic regression was used to build multimarker combinations for sepsis diagnosis. All marker combinations were assessed for their sepsis diagnostic performance and combinations which show significant better performance than Procalcitonin (PCT) were retained. Table 14 shows multimarker models with a significantly better performance than PCT for discriminating SIRS from all sepsis. PCT and IL-6 were measured using commercially available immunoassays whereas all other markers were measured using MASSterclass®, as described in example 1.

TABLE 14
Multimarker models which perform significantly better
than PCT for discriminating SIRS from sepsis

| Combination model | AUC (95% Cl) | p-value* |
|---|---|---|
| PCT + PRTN3 + GSHB | 0.75 (0.70-0.81) | 0.00 |
| PTX3 + PRTN3 + GSHB | 0.75 (0.69-0.81) | 0.03 |
| PCT + PRTN3 + VCAM1 | 0.74 (0.68-0.80) | 0.01 |
| PCT + PRTN3 + PSA3 | 0.73 (0.67-0.79) | 0.02 |
| PCT + PRTN3 + NID1 | 0.73 (0.67-0.79) | 0.02 |
| PCT + PRTN3 + GOLM1 | 0.73 (0.67-0.79) | 0.03 |
| PCT + PRTN3 + PTX3 | 0.72 (0.66-0.78) | 0.05 |
| PCT + GSHB | 0.72 (0.66-0.77) | 0.04 |
| PCT + GSHB + ATF6A | 0.73 (0.67-0.79) | 0.03 |
| PCT + GSHB + ICAM1 | 0.73 (0.67-0.79) | 0.02 |
| PCT + GSHB + | 0.73 (0.67-0.79) | 0.04 |
| donor_white_blood_cell_count | | |
| PCT + GSHB + PIGR | 0.73 (0.67-0.79) | 0.02 |
| PCT + GSHB + PTX3 | 0.73 (0.67-0.79) | 0.05 |
| PCT + GSHB + CALU | 0.73 (0.67-0.78) | 0.03 |
| PCT + VCAM1 + IL6 | 0.72 (0.66-0.78) | 0.05 |
| PCT + VCAM1 + EXT2 | 0.72 (0.66-0.78) | 0.04 |
| PCT + PHLD + EXT2 | 0.72 (0.66-0.77) | 0.04 |
| PCT + FGL1 + GOLM1 | 0.71 (0.65-0.78) | 0.04 |
| PCT + FGL1 + NID1 | 0.71 (0.65-0.77) | 0.04 |

*p-value for improvement over PCT

The triple marker combination of procalcitonin, proteinase 3 and glutathione synthetase (PCT+PRTN3+GSHB) showed the best performance for discriminating SIRS from sepsis compared to PCT as a standard single marker. It was further found that in the PCT+PRTN3+GSHB model, PCT could be replaced by Pentraxin-3 (PTX3) without loss of performance of the model.

The diagnostic performance of both these multimarker models in comparison to PCT and the dual marker models PCT+PRTN3 and PCT+GSHB is detailed in Table 15. The sensitivity and specificity of these models at a fixed cut-off of respectively 70% specificity and 70% sensitivity is shown in Table 16.

TABLE 15
Diagnostic performance (AUC (95% Cl) of multimarker models

| | | | | PCT + | PTX3 + |
|---|---|---|---|---|---|
| | | PCT + | PCT + | PRTN3 + | PRTN3 + |
| | PCT | PRTN3 | GSHB | GSHB | GSHB |
| SIRS - all | 0.68 (0.62-0.74) | 0.71 (0.65-0.77) | 0.72 (0.66-0.77)* | 0.75 (0.70-0.81)* | 0.75 (0.70-0.80)* |
| sepsis | | | | | |
| SIRS - mild | 0.59 (0.51-0.67) | 0.64 (0.56-0.71) | 0.64 (0.57-0.71) | 0.70 (0.63-0.76)* | 0.69 (0.62-0.76) |
| sepsis | | | | | |
| SIRS - severe | 0.76 (0.70-0.82) | 0.77 (0.71-0.84) | 0.78 (0.72-0.84) | 0.80 (0.74-0.85) | 0.80 (0.74-0.85) |
| sepsis | | | | | |

*improvement over PCT: p < 0.05

TABLE 16
Sensitivity and specificity of combination models
at respectively 70% specificity and 70% sensitivity

| | | | | PCT + | PTX3 + |
|---|---|---|---|---|---|
| | | PCT + | PCT + | PRTN3 + | PRTN3 + |
| | PCT | PRTN3 | GSHB | GSHB | GSHB |
| SIRS - all sepsis | | | | | |
| Sensitivity | 58% | 59% | 64% | 71% | 67% |
| Specificity | 54% | 58% | 60% | 71% | 65% |
| SIRS - mild sepsis | | | | | |
| Sensitivity | 45% | 49% | 50% | 65% | 60% |
| Specificity | 39% | 49% | 51% | 57% | 55% |
| SIRS - severe sepsis | | | | | |
| Sensitivity | 71% | 70% | 75% | 77% | 74% |
| Specificity | 71% | 71% | 75% | 78% | 76% |

1-36. (canceled)

37. A method for the diagnosis, prediction, prognosis and/or monitoring of a systemic inflammatory condition in a subject, wherein the method comprises measuring the quantity of any one or more markers selected from the group consisting of proteinase 3 (PRTN3), macrophage mannose receptor 1 (MRC1), exostoses (multiple) 2 (EXT2), interleukin 1 receptor type II (IL1R2), pentraxin 3 long (PTX3), mannosyl-oligosaccharide 1,2-alpha-mannosidase IA (MA1 A1), Acyl-CoA-binding protein (ACBP), vesicular integral-membrane protein VIP36 (LMAN2), neuronal acetylcholine receptor subunit alpha-7 (ACHA7), cyclic AMP-dependent transcription factor ATF-6 alpha (ATF6A), Beta-1,4-galactosyltransferase 1 (B4GT1), cathelicidin antimicrobial peptide (CAMP), Golgi membrane protein 1 (GOLM1), Nidogen-1 (NID1), matrix metallopeptidase 3 (MMP3), lipopolysaccharide-binding protein (LBP), fibulin 1 (FBLN1), polymeric-immunoglobulin receptor (PIGR), TIMP metalloproteinase inhibitor (TIMP1), glycosylphosphatidylinositol specific phospholipase D1 (PHLD), angiotensinogen (ANGT), carboxypeptidase N catalytic chain (CBPN), chitinase-3-like protein 1 (CH3L1), macrophage colony-stimulating factor 1 (CSF1), dystryglycan (DAG1), fibrillin-1 (FBN1), fibrinogen-like 1 (FGL1), glutathione synthetase (GSHB), intercellular adhesion molecule 1 (ICAM1), lumican (LUM), S100 calcium binding protein A9 (S10A9), serum amyloid A protein (SAA), serglycin (SRGN), Vascular cell adhesion protein 1 (VCAM1), calumenin (CALU), echinoderm microtubule associated protein like 3 (EMAL3), Rho GDP dissociation inhibitor beta (GDIR2), guanylate cyclase activator 2B (GUC2B), heat shock 70 kDa protein 8 (HSP7C), interleukin 13 receptor alpha 1 (I13R1), moesin (MOES), protein disulfide isomerase family A member 6 (PDIA6), proteasome subunit alpha type 3 (PSA3), protein tyrosine phosphatase receptor type G (PTPRG), S100 calcium binding protein A8 (S10A8), cathepsin G (CATG), and neutrophil elastase (ELNE), or a fragment thereof, in a sample from the subject.

38. The method according to claim 37, comprising the steps of:
(i) measuring the quantity of any one or more markers selected from the group consisting of PRTN3, MRC1, EXT2, IL1R2, PTX3, MA1A1, ACBP, LMAN2, ACHA7, ATF6A, B4GT1, CAMP, GOLM1, NID1, MMP3, LBP, FBLN1, PIGR, TIMP1, PHLD, ANGT, CBPN, CH3L1, CSF1, DAG1, FBN1, FGL1, GSHB, ICAM1, LUM, S10A9, SAA, SRGN, VCAM1, CALU, EMAL3, GDIR2, GUC2B, HSP7C, I13R1, MOES, PDIA6, PSA3, PTPRG, S10A8, CATG, and ELNE, or a fragment thereof, in the sample from the subject;
(ii) comparing the quantity of said one or more markers measured in (i) with a reference value of the quantity of said one or more markers, said reference value representing a known diagnosis, prediction and/or prognosis of the systemic inflammatory condition;
(iii) finding a deviation or no deviation of the quantity of said one or more markers measured in (i) from said reference value;
(iv) attributing said finding of deviation or no deviation to a particular diagnosis, prediction and/or prognosis of the systemic inflammatory condition in the subject.

39. The method according to claim 37 for monitoring the systemic inflammatory condition, preferably in the course of a medical treatment of the subject, comprising the steps of:
(i) measuring the quantity of any one or more markers selected from the group consisting of PRTN3, MRC1, EXT2, IL1R2, PTX3, MA1A1, ACBP, LMAN2, ACHA7, ATF6A, B4GT1, CAMP, GOLM1, NID1, MMP3, LBP, FBLN1, PIGR, TIMP1, PHLD, ANGT, CBPN, CH3L1, CSF1, DAG1, FBN1, FGL1, GSHB, ICAM1, LUM, S10A9, SAA, SRGN, VCAM1, CALU, EMAL3, GDIR2, GUC2B, HSP7C, I13R1, MOES, PDIA6, PSA3, PTPRG, S10A8, CATG, and ELNE, or a fragment thereof, in samples from the subject from two or more successive time points;
(ii) comparing the quantity of said one or more markers between the samples as measured in (i);
(iii) finding a deviation or no deviation of the quantity of said one or more markers between the samples as compared in (ii);
(iv) attributing said finding of deviation or no deviation to a change in the systemic inflammatory condition in the subject between the two or more successive time points.

40. The method according to claim 37, wherein (i) the method is for the diagnosis of whether a subject presenting with one or more signs of systemic inflammatory response syndrome (SIRS) has infection-free SIRS or has sepsis, or wherein (ii) the method is for the diagnosis, prediction and/or prognosis of the severity of the systemic inflammatory condition, preferably wherein the systemic inflammatory condition is SIRS or sepsis, in a subject, more preferably wherein said diagnosis, prediction and/or prognosis of the severity of the systemic inflammatory condition in the subject comprises the prediction of mortality in the subject or the prognosis that the systemic inflammatory condition will result in death of the subject, or comprises the diagnosis, prediction and/or prognosis of organ failure or multi-organ dysfunction syndrome in the subject.

41. The method according to claim 37, wherein:
the method is for the diagnosis of whether a subject presenting with one or more signs of SIRS has infection-free SIRS or has sepsis and comprises measuring the quantity of any one or more markers selected from the group consisting of PRTN3, MRC1, EXT2, IL1R2, PTX3, ACBP, ATF6A, B4GT1, GOLM1, NID1, LBP, FBLN1, TIMP1, CH3L1, CSF1, FGL1, ICAM1, LUM, S10A9, SAA, VCAM1, PSA3, PTPRG, S10A8, GSHB, PIGR, CALU, PHLD, CATG, and ELNE, or a fragment thereof, in a sample of the subject, or
the method is for the prediction of mortality in a subject having a systemic inflammatory condition, such as preferably having SIRS or sepsis, more preferably having sepsis, or for the prognosis that said systemic inflammatory condition, such as preferably SIRS or sepsis, more preferably sepsis, will result in death of the subject and comprises measuring the quantity of any one or more markers selected from the group consisting of MRC1, EXT2, PTX3, B4GT1, CAMP, GOLM1, TIMP1, PHLD, CH3L1, GSHB, ICAM1, VCAM1, HSP7C, PSA3 and PTPRG, or a fragment thereof, in a sample of the subject; or
the method is for the diagnosis, prediction and/or prognosis of organ failure or multi-organ dysfunction syndrome in a subject having a systemic inflammatory condition, such as preferably having SIRS or sepsis, more preferably having sepsis and comprises measuring the quantity of any one or more markers selected from the group consisting of MRC1, EXT2, IL1R2, PTX3, ACBP, B4GT1, NID1, TIMP1, CH3L1, FGL1, SAA and PSA3, or a fragment thereof, in a sample of the subject.

42. The method according to claim 37, wherein the method is for the diagnosis of whether a subject presenting with one or more signs of SIRS has infection-free SIRS or has sepsis and comprises measuring the quantity of any one or more of PRTN3, CATG, and ELNE, or a fragment thereof, preferably PRTN3, or a fragment thereof, in a sample of the subject.

43. The method according claim 37, wherein the method is for the prediction of mortality in a subject having a systemic inflammatory condition, such as preferably having SIRS or sepsis, more preferably having sepsis, or for the prognosis that said systemic inflammatory condition, such as preferably SIRS or sepsis, more preferably sepsis, will result in death of the subject and comprises measuring MRC1 or a fragment thereof in a sample of the subject.

44. The method according to claim 37, wherein the method is for the diagnosis, prediction and/or prognosis of organ failure or multi-organ dysfunction syndrome in a subject having a systemic inflammatory condition, such as preferably having SIRS or sepsis, more preferably having sepsis and comprises measuring any one or more of EXT2, IL1R2 and PTX3 or a fragment thereof in a sample of the subject.

45. The method according to claim 37 applied to monitor the effectiveness of therapy of the systemic inflammatory disease, or to decide on initiation, continuation or discontinuation (ending) of the therapy, wherein the therapy is preferably antibiotics therapy.

46. The method according to claim 37 further comprising measuring the presence or absence and/or quantity of one or more biomarkers selected from C-reactive protein (CRP), Procalcitonin (PCT), lactate, Cystatin C (CYTC), Neutrophil gelatinase-associated lipocalin (NGAL) and interleukin-6 (IL6), or a fragment thereof.

47. A test panel for the diagnosis, prediction, prognosis, and/or monitoring of a systemic inflammatory condition in a subject, preferably wherein the systemic inflammatory condition is sepsis or SIRS, more preferably wherein the systemic inflammatory condition is sepsis, the test panel comprising or consisting of: measurement of the level of Procalcitonin (PCT) or a fragment thereof in the subject; and measurement of the level of one or more markers selected from the group consisting of PRTN3, GSHB, PTX3, VCAM1, PSA3, NID1, GOLM1, ATF6A, ICAM1, PIGR, CALU, EXT2, PHLD, FGL1, IL1R2, CATG, and ELNE, or a fragment thereof, in the subject; optionally wherein the test panel comprises measurement of the level of PTX3 or a fragment thereof in the subject instead of or in addition to measurement of the level of PCT or a fragment thereof in the subject.

48. The test panel according to claim 47, further comprising the measurement of white blood cell (WBC) count in the subject.

49. The test panel according to claim 47, comprising measurement of the level of PCT or PTX3, or a fragment thereof, in the subject, and at least one and preferably both of measurement of the level of PRTN3 or a fragment thereof and measurement of the level of GSHB or a fragment thereof in the subject; optionally wherein the measurement of the level of PRTN3 or a fragment thereof is replaced or complemented by one or both of the measurement of the level of CATG or a fragment thereof and the measurement of the level of ELNE or a fragment thereof in the subject.

50. The test panel according to claim 47, further comprising measurement of the presence or absence and/or quantity of one or more biomarkers selected from C-reactive protein (CRP), lactate, Cystatin C (CYTC), Neutrophil gelatinase-associated lipocalin (NGAL) and interleukin-6 (IL6), or a fragment thereof, in the subject.

51. The test panel according to claim 47, comprising:
measurement of the level of PCT or a fragment thereof and measurement of the level of PRTN3 or a fragment thereof; or
measurement of the level of PCT or a fragment thereof and measurement of the level of GSHB or a fragment thereof; or
measurement of the level of PCT or a fragment thereof, measurement of the level of PRTN3 or a fragment thereof, and measurement of the level of GSHB or a fragment thereof; or
measurement of the level of PTX3 or a fragment thereof, measurement of the level of PRTN3 or a fragment thereof, and measurement of the level of GSHB or a fragment thereof;
optionally, wherein the measurement of the level of PRTN3 or a fragment thereof is replaced or complemented by one or both of the measurement of the level of CATG or a fragment thereof and the measurement of the level of ELNE or a fragment thereof.

52. The test panel according to claim 47, comprising:
measurement of the level of PCT or a fragment thereof, measurement of the level of PRTN3 or a fragment thereof, and measurement of the level of VCAM1 or a fragment thereof; or
measurement of the level of PCT or a fragment thereof, measurement of the level of PRTN3 or a fragment thereof, and measurement of the level of PSA3 or a fragment thereof; or
measurement of the level of PCT or a fragment thereof, measurement of the level of PRTN3 or a fragment thereof, and measurement of the level of NID1 or a fragment thereof; or
measurement of the level of PCT or a fragment thereof, measurement of the level of PRTN3 or a fragment thereof, and measurement of the level of GOLM1 or a fragment thereof; or
measurement of the level of PCT or a fragment thereof, measurement of the level of PRTN3 or a fragment thereof, and measurement of the level of PTX3 or a fragment thereof; or
measurement of the level of PCT or a fragment thereof, measurement of the level of GSHB or a fragment thereof, and measurement of the level of ATF6A or a fragment thereof; or
measurement of the level of PCT or a fragment thereof, measurement of the level of GSHB or a fragment thereof, and measurement of the level of ICAM1 or a fragment thereof; or
measurement of the level of PCT or a fragment thereof, measurement of the level of GSHB or a fragment thereof, and measurement of WBC; or
measurement of the level of PCT or a fragment thereof, measurement of the level of GSHB or a fragment thereof, and measurement of the level of PIGR or a fragment thereof; or
measurement of the level of PCT or a fragment thereof, measurement of the level of GSHB or a fragment thereof, and measurement of the level of PTX3 or a fragment thereof; or
measurement of the level of PCT or a fragment thereof, measurement of the level of GSHB or a fragment thereof, and measurement of the level of CALU or a fragment thereof; or
measurement of the level of PCT or a fragment thereof, measurement of the level of VCAM1 or a fragment thereof, and measurement of the level of IL6 or a fragment thereof; or
measurement of the level of PCT or a fragment thereof, measurement of the level of VCAM1 or a fragment thereof, and measurement of the level of EXT2 or a fragment thereof; or
measurement of the level of PCT or a fragment thereof, measurement of the level of PHLD or a fragment thereof, and measurement of the level of EXT2 or a fragment thereof; or
measurement of the level of PCT or a fragment thereof, measurement of the level of FGL1 or a fragment thereof, and measurement of the level of GOLM1 or a fragment thereof; or
measurement of the level of PCT or a fragment thereof, measurement of the level of FGL1 or a fragment thereof, and measurement of the level of NID1 or a fragment thereof;
optionally wherein the measurement of the level of PRTN3 or a fragment thereof is replaced or complemented by one or both of the measurement of the level of CATG or a fragment thereof and the measurement of the level of ELNE or a fragment thereof in the subject.

53. A method for the diagnosis, prediction, prognosis and/or monitoring of a systemic inflammatory condition in a subject, wherein the method comprises testing or evaluating in the subject the test panel as defined claim 47.

54. The method according to claim 53, wherein the method is:
for the diagnosis of sepsis, particularly for diagnosis whether a subject presenting with one or more signs of systemic inflammatory response syndrome (SIRS) has infection-free SIRS or has sepsis; or
for the diagnosis, prediction and/or prognosis of the severity of the systemic inflammatory condition, preferably wherein the systemic inflammatory condition is SIRS or sepsis, in a subject, preferably, wherein said diagnosis, prediction and/or prognosis of the severity of the systemic inflammatory condition in the subject comprises the prediction of mortality in the subject or the prognosis that the systemic inflammatory condition will result in death of the subject, or comprises the diagnosis, prediction and/or prognosis of organ failure or multi-organ dysfunction syndrome in the subject;
for monitoring of a systemic inflammatory condition in a subject, preferably in the course of a medical treatment of the subject, preferably wherein the systemic inflammatory condition is sepsis or SIRS, more preferably wherein the systemic inflammatory condition is sepsis.

55. The method according to claim 37, wherein the subject is a critically ill patient.

56. The method according to claim 37, wherein the subject is known or suspected to have a systemic inflammatory condition, such as sepsis or SIRS.

57. The method according to claim 37, wherein the one or more biomarkers is protein-, polypeptide- or peptide-based.

58. The method according to claim 37, wherein the quantity of said one or more markers, or a fragment thereof, is measured using an immunoassay technology, using a mass spectrometry analysis method, using a chromatography method, or using a combination of said methods.


SEQUENCE LISTING (35 sequences)

SEQ ID NO: 1 - protein, 256 aa, Homo sapiens
MAHRPPSPALASVLLALLLSGAARAAEIVGGHEAQPHSRPYMASLQMRGNPGSHFCGGTLIHPSFVLTAAHCLRDIPQRLVNVVLGAHNVRTQEPTQQHFSVAQVFLNNYDAENKLNDVLLIQLSSPANLSASVATVQLPQQDQPVPHGTQCLAMGWGRVGAHDPPAQVLQELNVTVVTFFCRPHNICTFVPRRKAGICFGDSGGPLICDGIIQGIDSFVIWGCATRLFPDFFTRVALYVDWIRSTLRRVEAKGRP

SEQ ID NO: 2 - protein, 8 aa, Homo sapiens
LFPDFFTR

SEQ ID NO: 3 - protein, 1456 aa, Homo sapiens [sequence omitted]

SEQ ID NO: 4 - protein, 11 aa, Homo sapiens
SQGPEIVEVEK

SEQ ID NO: 5 - protein, 381 aa, Homo sapiens
MHLLAILFCALWSAVLAENSDDYDLMYVNLDNEIDNGLHPTEDPTPCACGQEHSEWDKLFIMLENSQMRERMLLQATDDVLRGELQRLREELGRLAESLARPCAPGAPAEARLTSALDELLQATRDAGRRLARMEGAEAQRPEEAGRALAAVLEELRQTRADLHAVQGWAARSWLPAGCETAILFPMRSKKIFGSVHPVRPMRLESFSACIWVKATDVLNKTILFSYGTKRNPYEIQLYLSYQSIVFVVGGEENKLVAEAMVSLGRWTHLCGTWNSEEGLTSLWVNGELAATTVEMATGHIVPEGGILQIGQEKNGCCVGGGFDETLAFSGRLTGFNIWDSVLSNEEIRETGGAESCHIRGNIVGWGVTEIQPHGGAQYVS

SEQ ID NO: 6 - protein, 10 aa, Homo sapiens
ALAAVLEELR

SEQ ID NO: 7 - protein, 398 aa, Homo sapiens
MLRLYVLVMGVSAFTLQPAAHTGAARSCRFRGRHYKREFRLEGEPVALRCPQVPYWLWASVSPRINLTWHKNDSARTVPGEEETRMWAQDGALWLLPALQEDSGTYVCTTRNASYCDKMSIELRVFENTDAFLPFISYPQILTLSTSGVLVCPDLSEFTRDKTDVKIQWYKDSLLLDKDNEKFLSVRGTTHLLVHDVALEDAGYYRCVLTFAHEGQQYNITRSIELRIKKKKEETIPVIISPLKTISASLGSRLTIPCKVFLGTGTPLTTMLWWTANDTHIESAYPGGRVTEGPRQEYSENNENYIEVPLIFDPVTREDLHMDFKCVVHNTLSFQTLRTTVKEASSTFSWGIVLAPLSLAFLVLGGIWMHRRCKHRTGKADGLTVLWPHHQDFQSYPK

SEQ ID NO: 8 - protein, 12 aa, Homo sapiens
EETIPVIISPLK

SEQ ID NO: 9 - protein, 9 aa, Homo sapiens
LEGEPVALR

SEQ ID NO: 10 - protein, 718 aa, Homo sapiens
MCASVKYNIRGPALIPRMKTKHRIYYITLFSIVLLGLIATGMFQFWPHSIESSNDWNVEKRSIRDVPVVRLPADSPIPERGDLSCRMHTCFDVYRCGFNPKNKIKVYIYALKKYVDDFGVSVSNTISREYNELLMAISDSDYYTDDINRACLFVPSIDVLNQNTLRIKETAQAMAQLSRWDRGTNHLLFNMLPGGPPDYNTALDVPRDRALLAGGGFSTWTYRQGYDVSIPVYSPLSAEVDLPEKGPGPRQYFLLSSQVGLHPEYREDLEALQVKHGESVLVLDKCTNLSEGVLSVRKRCHKHQVFDYPQVLQEATFCVVLRGARLGQAVLSDVLQAGCVPVVIADSYILPFSEVLDWKRASVVVPEEKMSDVYSILQSIPQRQIEEMQRQARWFWEAYFQSIKAIALATLQIINDRIYPYAAISYEEWNDPPAVKWGSVSNPLFLPLIPPQSQGFTAIVLTYDRVESLFRVITEVSKVPSLSKLLVVWNNQNKNPPEDSLWPKIRVPLKVVRTAENKLSNRFFPYDEIETEAVLAIDDDIIMLTSDELQFGYEVWREFPDRLVGYPGRLHLWDHEMNKWKYESEWTNEVSMVLTGAAFYHKYFNYLYTYKMPGDIKNWVDAHMNCEDIAMNFLVANVTGKAVIKVTPRKKFKCPECTAIDGLSLDQTHMVERSECINKFASVFGTMPLKVVEHRADPVLYKDDFPEKLKSFPNIGSL

SEQ ID NO: 11 - protein, 9 aa, Homo sapiens
EDLEALQVK

SEQ ID NO: 12 - protein, 10 aa, Homo sapiens
LPADSPIPER

SEQ ID NO: 13 - protein, 474 aa, Homo sapiens
MATNWGSLLQDKQQLEELARQAVDRALAEGVLLRTSQEPTSSEVVSYAPFTLFPSLVPSALLEQAYAVQMDFNLLVDAVSQNAAFLEQTLSSTIKQDDFTARLFDIHKQVLKEGIAQTVFLGLNRSDYMFQRSADGSPALKQIEINTISASFGGLASRTPAVHRHVLSVLSKTKEAGKILSNNPSKGLALGIAKAWELYGSPNALVLLIAQEKERNIFDQRAIENELLARNIHVIRRTFEDISEKGSLDQDRRLFVDGQEIAVVYFRDGYMPRQYSLQNWEARLLLERSHAAKCPDIATQLAGTKKVQQELSRPGMLEMLLPGQPEAVARLRATFAGLYSLDVGEEGDQAIAEALAAPSRFVLKPQREGGGNNLYGEEMVQALKQLKDSEERASYILMEKIEPEPFENCLLRPGSPARVVQCISELGIFGVYVRQEKTLVMNKHVGHLLRTKAIEHADGGVAAGVAVLDNPYPV

SEQ ID NO: 14 - protein, 9 aa, Homo sapiens
AIENELLAR

SEQ ID NO: 15 - protein, 8 aa, Homo sapiens
TFEDISEK

SEQ ID NO: 16 - protein, 739 aa, Homo sapiens
MPGKMVVILGASNILWIMFAASQAFKIETTPESRYLAQIGDSVSLTCSTTGCESPFFSWRTQIDSPLNGKVTNEGTTSTLTMNPVSFGNEHSYLCTATCESRKLEKGIQVEIYSFPKDPEIHLSGPLEAGKPITVKCSVADVYPFDRLEIDLLKGDHLMKSQEFLEDADRKSLETKSLEVTFTPVIEDIGKVLVCRAKLHIDEMDSVPTVRQAVKELQVYISPKNTVISVNPSTKLQEGGSVTMTCSSEGLPAPEIFWSKKLDNGNLQHLSGNATLTLIAMRMEDSGIYVCEGVNLIGKNRKEVELIVQEKPFTVEISPGPRIAAQIGDSVMLTCSVMGCESPSFSWRTQIDSPLSGKVRSEGTNSTLTLSPVSFENEHSYLCTVTCGHKKLEKGIQVELYSFPRDPEIEMSGGLVNGSSVTVSCKVPSVYPLDRLEIELLKGETILENIEFLEDTDMKSLENKSLEMTFIPTIEDTGKALVCQAKLHIDDMEFEPKQRQSTQTLYVNVAPRDTTVLVSPSSILEEGSSVNMTCLSQGFPAPKILWSRQLPNGELQPLSENATLTLISTKMEDSGVYLCEGINQAGRSRKEVELIIQVTPKDIKLTAFPSESVKEGDTVIISCTCGNVPETWIILKKKAETGDTVLKSIDGAYTIRKAQLKDAGVYECESKNKVGSQLRSLTLDVQGRENNKDYFSPELLVLYFASSLIIPAIGMIIYFARKANMKGSYSLVEAQKSKV

SEQ ID NO: 17 - protein, 15 aa, Homo sapiens
SLEVTFTPVIEDIGK

SEQ ID NO: 18 - protein, 255 aa, Homo sapiens
MSSIGTGYDLSASTFSPDGRVFQVEYAMKAVENSSTAIGIRCKDGVVFGVEKLVLSKLYEEGSNKRLFNVDRHVGMAVAGLLADARSLADIAREEASNFRSNFGYNIPLKHLADRVAMYVHAYTLYSAVRPFGCSFMLGSYSVNDGAQLYMIDPSGVSYGYWGCAIGKARQAAKTEIEKLQMKEMTCRDIVKEVAKIIYIVHDEVKDKAFELELSWVGELTNGRHEIVPKDIREEAEKYAKESLKEEDESDDDNM

SEQ ID NO: 19 - protein, 9 aa, Homo sapiens
DGVVFGVEK

SEQ ID NO: 20 - protein, 1247 aa, Homo sapiens
MLASSSRIRAAWTRALLLPLLLAGPVGCLSRQELFPFGPGQGDLELEDGDDFVSPALELSGALRFYDRSDIDAVYVTTNGIIATSEPPAKESHPGLFPPTFGAVAPFLADLDTTDGLGKVYYREDLSPSITQRAAECVHRGFPEISFQPSSAVVVTWESVAPYQGPSRDPDQKGKRNTFQAVLASSDSSSYAIFLYPEDGLQFHTTFSKKENNQVPAVVAFSQGSVGFLWKSNGAYNIFANDRESVENLAKSSNSGQQGVWVFEIGSPATTNGVVPADVILGTEDGAEYDDEDEDYDLATTRLGLEDVGTTPFSYKALRRGGADTYSVPSVLSPRRAATERPLGPPTERTRSFQLAVETFHQQHPQVIDVDEVEETGVVFSYNTDSRQTCANNRHQCSVHAECRDYATGFCCSCVAGYTGNGRQCVAEGSPQRVNGKVKGRIFVGSSQVPIVFENTDLHSYVVMNHGRSYTAISTIPETVGYSLLPLAPVGGIIGWMFAVEQDGFKNGFSITGGEFTRQAEVTFVGHPGNLVIKQRFSGIDEHGHLTIDTELEGRVPQIPFGSSVHIEPYTELYHYSTSVITSSSTREYTVTEPERDGASPSRIYTYQWRQTITFQECVHDDSRPALPSTQQLSVDSVFVLYNQEEKILRYALSNSIGPVREGSPDALQNPCYIGTHGCDTNAACRPGPRTQFTCECSIGFRGDGRTCYDIDECSEQPSVCGSHTICNNHPGTFRCECVEGYQFSDEGTCVAVVDQRPINYCETGLHNCDIPQRAQCIYTGGSSYTCSCLPGFSGDGQACQDVDECQPSRCHPDAFCYNTPGSFTCQCKPGYQGDGFRCVPGEVEKTRCQHEREHILGAAGATDPQRPIPPGLFVPECDAHGHYAPTQCHGSTGYCWCVDRDGREVEGTRTRPGMTPPCLSTVAPPIHQGPAVPTAVIPLPPGTHLLFAQTGKIERLPLEGNTMRKTEAKAFLHVPAKVIIGLAFDCVDKMVYWTDITEPSIGRASLHGGEPTTIIRQDLGSPEGIAVDHLGRNIFWTDSNLDRIEVAKLDGTQRRVLFETDLVNPRGIVTDSVRGNLYWTDWNRDNPKIETSYMDGTNRRILVQDDLGLPNGLTFDAFSSQLCWVDAGTNRAECLNPSQPSRRKALEGLQYPFAVTSYGKNLYFTDWKMNSVVALDLAISKETDAFQPHKQTRLYGITTALSQCPQGHNYCSVNNGGCTHLCLATPGSRTCRCPDNTLGVDCIEQK

SEQ ID NO: 21 - protein, 11 aa, Homo sapiens
VLFETDLVNPR

SEQ ID NO: 22 - protein, 401 aa, Homo sapiens
MMGLGNGRRSMKSPPLVLAALVACIIVLGFNYWIASSRSVDLQTRIMELEGRVRRAAAERGAVELKKNEFQGELEKQREQLDKIQSSHNFQLESVNKLYQDEKAVLVNNITTGERLIRVLQDQLKTLQRNYGRLQQDVLQFQKNQTNLERKFSYDLSQCINQMKEVKEQCEERIEEVTKKGNEAVASRDLSENNDQRQQLQALSEPQPRLQAAGLPHTEVPQGKGNVLGNSKSQTPAPSSEVVLDSKRQVEKEETNEIQVVNEEPQRDRLPQEPGREQVVEDRPVGGRGFGGAGELGQTPQVQAALSVSQENPEMEGPERDQLVIPDGQEEEQEAAGEGRNQQKLRGEDDYNMDENEAESETDKQAALAGNDRNIDVFNVEDQKRDTINLLDQREKRNHTL

SEQ ID NO: 23 - protein, 9 aa, Homo sapiens
DTINLLDQR

SEQ ID NO: 24 - protein, 670 aa, Homo sapiens
MGEPAGVAGTMESPFSPGLFHRLDEDWDSALFAELGYFTDTDELQLEAANETYENNFDNLDFDLDLMPWESDIWDINNQICTVKDIKAEPQPLSPASSSYSVSSPRSVDSYSSTQHVPEELDLSSSSQMSPLSLYGENSNSLSSAEPLKEDKPVTGPRNKTENGLTPKKKIQVNSKPSIQPKPLLLPAAPKTQTNSSVPAKTIIIQTVPTLMPLAKQQPIISLQPAPTKGQTVLLSQPTVVQLQAPGVLPSAQPVLAVAGGVTQLPNHVVNVVPAPSANSPVNGKLSVTKPVLQSTMRNVGSDIAVLRRQQRMIKNRESACQSRKKKKEYMLGLEARLKAALSENEQLKKENGTLKRQLDEVVSENQRLKVPSPKRRVVCVMIVLAFIILNYGPMSMLEQDSRRMNPSVSPANQRRHLLGFSAKEAQDTSDGIIQKNSYRYDHSVSNDKALMVLTEEPLLYIPPPPCQPLINTTESLRLNHELRGWVHRHEVERTKSRRMTNNQQKTRILQGALEQGSNSQLMAVQYTETTSSISRNSGSELQVYYASPRSYQDFFEAIRRRGDTFYVVSFRRDHLLLPATTHNKTTRPKMSIVLPAININENVINGQDYEVMMQIDCQVMDTRILHIKSSSVPPYLRDQQRNQTNTFFGSPPAATEATHVVSTIPESLQ

SEQ ID NO: 25 - protein, 12 aa, Homo sapiens
EAQDTSDGIIQK

SEQ ID NO: 26 - protein, 532 aa, Homo sapiens
MAPSSPRPALPALLVLLGALFPGPGNAQTSVSPSKVILPRGGSVLVTCSTSCDQPKLLGIETPLPKKELLLPGNNRKVYELSNVQEDSQPMCYSNCPDGQSTAKTFLTVYWTPERVELAPLPSWQPVGKNLTLRCQVEGGAPRANLTVVLLRGEKELKREPAVGEPAEVTTTVLVRRDHHGANFSCRTELDLRPQGLELFENTSAPYQLQTFVLPATPPQLVSPRVLEVDTQGTVVCSLDGLFPVSEAQVHLALGDQRLNPTVTYGNDSFSAKASVSVTAEDEGTQRLTCAVILGNQSQETLQTVTIYSFPAPNVILTKPEVSEGTEVTVKCEAHPRAKVTLNGVPAQPLGPRAQLLLKATPEDNGRSFSCSATLEVAGQLIHKNQTRELRVLYGPRLDERDCPGNWTWPENSQQTPMCQAWGNPLPELKCLKDGTFPLPIGESVTVTRDLEGTYLCRARSTQGEVTRKVTVNVLSPRYEIVIITVVAAAVIMGTAGLSTYLYNRQRKIKKYRLQQAQKGTPMKPNTQATPP

SEQ ID NO: 27 - protein, 17 aa, Homo sapiens
EPAVGEPAEVTTTVLVR

SEQ ID NO: 28 - protein, 764 aa, Homo sapiens
MLLFVLTCLLAVFPAISTKSPIFGPEEVNSVEGNSVSITCYYPPTSVNRHTRKYWCRQGARGGCITLISSEGYVSSKYAGRANLTNFPENGTFVVNIAQLSQDDSGRYKCGLGINSRGLSFDVSLEVSQGPGLLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGGSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPRLFAEEKAVADTRDQADGSRASVDSGSSEEQGGSSRALVSTLVPLGLVLAVGAVAVGVARARHRKNVDRVSIRSYRTDISMSDFENSREFGANDNMGASSITQETSLGGKEEFVATTESTTETKEPKKAKRSSKEEAEMAYKDFLLQSSTVAAEAQDGPQEA

SEQ ID NO: 29 - protein, 9 aa, Homo sapiens
IIEGEPNLK

SEQ ID NO: 30 - protein, 315 aa, Homo sapiens
MDLRQFLMCLSLCTAFALSKPTEKKDRVHHEPQLSDKVHNDAQSFDYDHDAFLGAEEAKTFDQLTPEESKERLGKIVSKIDGDKDGFVTVDELKDWIKFAQKRWIYEDVERQWKGHDLNEDGLVSWEEYKNATYGYVLDDPDPDDGFNYKQMMVRDERRFKMADKDGDLIATKEEFTAFLHPEEYDYMKDIVVQETMEDIDKNADGFIDLEEYIGDMYSHDGNTDEPEWVKTEREQFVEFRDKNRDGKMDKEETKDWILPSDYDHAEAEARHLVYESDQNKDGKLTKEEIVDKYDLFVGSQATDFGEALVRHDEF

SEQ ID NO: 31 - protein, 8 aa, Homo sapiens
WIYEDVER

SEQ ID NO: 32 - protein, 840 aa, Homo sapiens
MSAFRLWPGLLIMLGSLCHRGSPCGLSTHVEIGHRALEFLQLHNGRVNYRELLLEHQDAYQAGIVFPDCFYPSICKGGKFHDVSESTHWTPFLNASVHYIRENYPLPWEKDTEKLVAFLFGITSHMAADVSWHSLGLEQGFLRTMGAIDFHGSYSEAHSAGDFGGDVLSQFEFNFNYLARRWYVPVKDLLGIYEKLYGRKVITENVIVDCSHIQFLEMYGEMLAVSKLYPTYSTKSPFLVEQFQEYFLGGLDDMAFWSTNIYHLTSFMLENGTSDCNLPENPLFIACGGQQNHTQGSKMQKNDFHRNLTTSLTESVDRNINYTERGVFFSVNSWTPDSMSFIYKALERNIRTMFIGGSQLSQKHVSSPLASYFLSFPYARLGWAMTSADLNQDGHGDLVVGAPGYSRPGHIHIGRVYLIYGNDLGLPPVDLDLDKEAHRILEGFQPSGRFGSALAVLDFNVDGVPDLAVGAPSVGSEQLTYKGAVYVYFGSKQGGMSSSPNITISCQDIYCNLGWTLLAADVNGDSEPDLVIGSPFAPGGGKQKGIVAAFYSGPSLSDKEKLNVEAANWTVRGEEDFSWFGYSLHGVTVDNRTLLLVGSPTWKNASRLGHLLHIRDEKKSLGRVYGYFPPNGQSWFTISGDKAMGKLGTSLSSGHVLMNGTLKQVLLVGAPTYDDVSKVAFLTVTLHQGGATRMYALTSDAQPLLLSTFSGDRRFSRFGGVLHLSDLDDDGLDEIIMAAPLRIADVTSGLIGGEDGRVYVYNGKETTLGDMTGKCKSWITPCPEEKAQYVLISPEASSRFGSSLITVRSKAKNQVVIAAGRSSLGARLSGALHVYSLGSD

SEQ ID NO: 33 - protein, 15 aa, Homo sapiens
IADVTSGLIGGEDGR

SEQ ID NO: 34 - protein, 312 aa, Homo sapiens
MAKVFSFILVTTALTMGREISALEDCAQEQMRLRAQVRLLETRVKQQQVKIKQLLQENEVQFLDKGDENTVIDLGSKRQYADCSEIFNDGYKLSGFYKIKPLQSPAEFSVYCDMSDGGGWTVIQRRSDGSENFNRGWKDYENGFGNFVQKHGEYWLGNKNLHFLTTQEDYTLKIDLADFEKNSRYAQYKNFKVGDEKNFYELNIGEYSGTAGDSLAGNFHPEVQWWASHQRMKFSTWDRDHDNYEGNCAEEDQSGWWFNRCHSANLNGVYYSGPYTAKTDNGIVWYTWHGWWYSLKSVVMKIRPNDFIPNVI

SEQ ID NO: 35 - protein, 12 aa, Homo sapiens
DYENGFGNFVQK